(19) United States
(12) Patent Application Publication
(10) Pub. No.: US 2026/0365692 A1
(43) Pub. Date: Nov. 19, 2026

(54) COMPOSITIONS COMPRISING PROTEINS DISSOLVED IN FLUORINATED SOLVENTS, AND METHODS OF MAKING AND USING THE SAME

(71) Applicant: The Penn State Research Foundation, University Park, PA (US)

(72) Inventors: Scott Medina, University Park, PA (US); Harminder Singh, University Park, PA (US)

(21) Appl. No.: 19/118,655

(22) PCT Filed: Oct. 6, 2023
(86) PCT No.: PCT/US2023/034700

§ 371 Date: Apr. 4, 2025

Related U.S. Application Data
(60) Provisional application No. 63/414,209, filed on Oct. 7, 2022.

(51) Int. Cl. C07K 1/02; C07K 1/113

(52) U.S. Cl. CPC C07K 1/02; C07K 1/113

(57) ABSTRACT

The present disclosure relates to perfluorinated compounds and methods of using said perfluorinated compounds to coat the surfaces of proteins to enable protein dispersion into fluorous solvents. Fluorochemical coatings could allow proteins to be dispersed within perfluorocarbon solvents, eliminating the need for cold storage and producing a product that does not require liquid water and has a reduced risk of contamination with bacteria or viruses that require aqueous environments for survival.

### CROSS-REFERENCE TO RELATED APPLICATIONS

The present application claims priority to U.S. Provisional Application No. 63/414,209 filed on Oct. 7, 2022, which is incorporated herein by reference in its entirety.

### GOVERNMENT SUPPORT

This invention was made with government support under Grant No. D21AP10182 awarded by the U.S. Department of the Interior/Defense Advanced Research Project Agency (MIPR #HR0011153917). The Government has certain rights in the invention.

### FIELD

The present disclosure relates to perfluorinated compounds and methods of using perfluorinated compounds to coat the surfaces of proteins to enable protein dispersion into fluorous solvents.

### BACKGROUND

Protein therapeutics must be produced, shipped and stored in refrigerated or frozen conditions. This is not only costly but complicates the deployment of protein therapeutics to regions with limited cold chain infrastructure. Fluorochemical coatings could allow proteins to be dispersed within perfluorocarbon solvents, eliminating the need for cold storage and producing a product that does not require liquid water and has a reduced risk of contamination with bacteria or viruses that require aqueous environments for survival. There is a need in the art for coatings that can be applied to protein therapeutics to eliminate the necessity of cold storage and reduce the risk of contamination with micro-organism. A similar need exists for other therapeutic candidates like RNA, DNA, and peptide. The present embodiments fulfill this need as well as others.

### SUMMARY

In some embodiments, a composition is provided, the composition comprising a fluorinated solvent, a dispersant compound, and a protein of interest, wherein: the dispersant compound interacts with the protein of interest; and the protein of interest is dissolved in the fluorinated solvent.

In some embodiments, the fluorinated solvent is selected from the group consisting of perfluoropentane, perfluorohexane, perfluoroheptane, perfluorooctane, perfluoromethylcyclohexane, perfluoro-1,3-dimethylcyclohexane, and perfluorodecalin. In some embodiments, the fluorinated solvent is perfluorohexane. In some embodiments, the fluorinated solvent is perfluorooctane.

In some embodiments, the dispersant compound is perfluorinated. In some embodiments, the dispersant compound comprises Formula II:

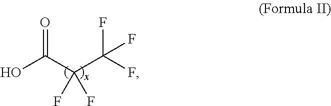

wherein x is 6, 7, 8, 9, 10, 12, or 16. In some embodiments, x is 7 (perfluoronanonic acid). In some embodiments, the dispersant compound comprises Formula III:

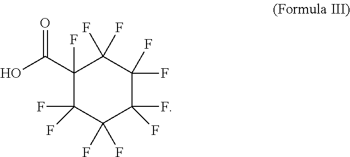 [SMILES: O=C(O)C1(F)C(F)(F)C(F)(F)C(F)(F)C(F)(F)C1(F)F]

In some embodiments, the dispersant compound comprises Formula V:

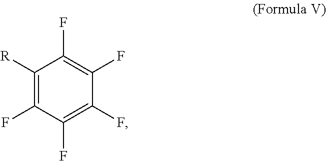

wherein R is OH or COH. In some embodiments, the dispersant compound comprises Formula VI:

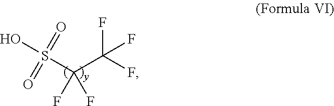

wherein y is 7. In some embodiments, the dispersant compound comprises Formula VIII:

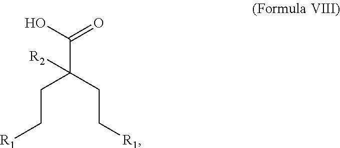

wherein R₁ is C₆F₁₃, C₈F₁₇, or C₄F₉, and wherein R₂ is CN, H, or COOH. In some embodiments, R₁ is C₆F₁₃ and R₂ is H.

In some embodiments, the dispersant compound interacts with the protein of interest via hydrogen bonding with nitrogen donors present in the protein backbone and amino acid side chains of the protein of interest. In some embodiments, the protein of interest comprises a non-native secondary structure.

In some embodiments, the fluorinated solvent is perfluorohexane and the dispersant compound is perfluoronanonic acid.

In some embodiments, the fluorinated solvent is perfluorooctane and the dispersant compound comprises Formula VIII:

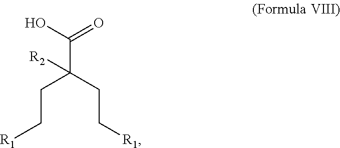

wherein R₁ is C₆F₁₃ and R₂ is H.

In some embodiments, a method of dissolving a protein of interest in a fluorinated solvent is provided, the method comprising: contacting the protein of interest with a dispersant compound and dissolving the protein of interest and dispersant compound in the fluorinated solvent.

In some embodiments, the method of dissolving a protein of interest in a fluorinated solvent comprises a fluorinated solvent selected from the group consisting of perfluoropentane, perfluorohexane, perfluoroheptane, perfluorooctane, perfluoromethylcyclohexane, perfluoro-1,3-dimethylcyclohexane, and perfluorodecalin. In some embodiments, the fluorinated solvent is perfluorohexane. In some embodiments, the fluorinated solvent is perfluorooctane.

In some embodiments, the method of dissolving a protein of interest in a fluorinated solvent comprises a dispersant compound, wherein the dispersant compound is perfluorinated. In some embodiments, the dispersant compound comprises Formula II:

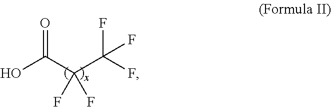

wherein x is 6, 7, 8, 9, 10, 12, or 16. In some embodiments, x is 7 (perfluoronanonic acid). In some embodiments, the dispersant compound comprises Formula III:

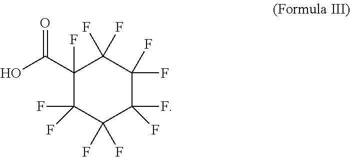 [SMILES: O=C(O)C1(F)C(F)(F)C(F)(F)C(F)(F)C(F)(F)C1(F)F]

In some embodiments, the dispersant compound comprises Formula V.

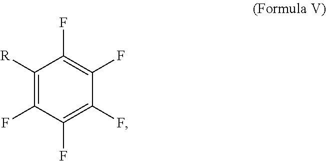

wherein R is OH or COH. In some embodiments, the dispersant compound comprises Formula VI:

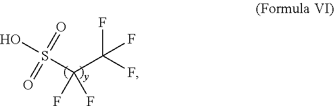

wherein y is 7. In some embodiments,
the dispersant compound comprises Formula VIII:

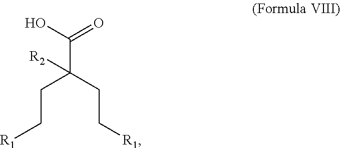

wherein R₁ is C₆F₁₃, C₈F₁₇, or C₄F₉, and wherein R₂ is CN, H, or COOH. In some embodiments, R₁ is C₆F₁₃ and R₂ is H.

In some embodiments, the method of dissolving a protein of interest in a fluorinated solvent comprises a dispersant compound, wherein the dispersant compound interacts with the protein of interest via hydrogen bonding with nitrogen donors present in the protein backbone and amino acid side chains of the protein of interest. In some embodiments, the protein of interest comprises a non-native secondary structure.

In some embodiments, the method of dissolving a protein of interest in a fluorinated solvent comprises a fluorinated solvent, wherein the fluorinated solvent is perfluorohexane and the dispersant compound is perfluoronanonic acid. In some embodiments, the fluorinated solvent is perfluorooctane and the dispersant compound comprises Formula VIII:

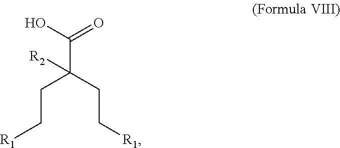

wherein R₁ is C₆F₁₃ and R₂ is H.

### BRIEF DESCRIPTION OF THE DRAWINGS

FIG. 1 depicts structures of fluorochemical compounds 0, 1, and 2, which were based on the Formula I with 4, 6, and 12 repeats (indicated as ‘v’ in Formula I), respectively. The compounds were screened for protein binding avidity.

FIGS. 2A-2B depicts the structure of fluorochemical compounds 3-7 (FIG. 2A) and compounds 8-12 (FIG. 2B), which were based off Formula II with 0, 1, 3, 6, 7, 8, 9, 10, 12, or 16 repeats (indicated as ‘x’ in Formula II) for each compound 3-12, respectively. The compounds were screened for protein binding avidity.

FIG. 3 depicts the structure of fluorochemical compound 13, which was based on the Formula III. The compound was screened for protein binding avidity.

FIG. 4 depicts the structure of fluorochemical compound 14, which was based on the Formula IV. The structure of the bound FMOC group is shown at the bottom of the figure. The compound was screened for protein binding avidity.

FIG. 5 depicts structures of fluorochemical compounds 15 and 16, which were based on the Formula V, where the R group was OH for compound 15 and COH for compound 16. The compounds were screened for protein binding avidity.

FIG. 6 depicts the structure of fluorochemical compound 17, which was based on the Formula VI with 7 repeats (indicated as ‘y’ in Formula VI). The compound was screened for protein binding avidity.

FIG. 7 depicts structures of fluorochemical compounds 18, 19, and 20, which were based on the Formula VII with 2, 6, and 7 repeats (indicated as ‘z’ in Formula VII), respectively. The compounds were screened for protein binding avidity.

FIGS. 8A-8G depict fluorochemical-mediated dispersion of test proteins hemoglobin (Hb, FIG. 8A), green fluorescent protein (GFP, FIG. 8B), bovine serum albumin (BSA, FIG. 8C), β-Galactosidase (β-Gal, FIG. 8D), rabbit serum immunoglobulin G (IgG, FIG. 8E), bovine apo-transferrin (TFN, FIG. 8F), and urease (URE, FIG. 8G) into perfluorohexane. Results are displayed as percent soluble protein relative to initial loading. The compound ‘0’ indicates a control sample absent of an additive. The crystal structure of each protein candidate tested is shown in the inset of each of FIGS. 8A-8G. Only the rabbit IgG Fc fragment is shown.

FIGS. 9A-9C depict the relationship between protein dispersion efficiency (FIG. 9A) and length of perfluoroalkyl tail of tested perfluorinated carboxylic acids (compounds 3-12), as well as predicted log P (FIG. 9B) and pKa (FIG. 9C) of all relevant fluorochemical compounds.

FIGS. 10A-10F depict the comparison of PFNA-mediated fluorous phase separation (dispersion) and protein macromolecular properties, including molecular weight (FIG. 10A), solvent-accessible surface area (FIG. 10B) (SASA), hydrodynamic radius (FIG. 10C), isoelectric point (pI) (FIG. 10D), α-helix content (FIG. 10E), and β-sheet content (FIG. 10F). Dashed lines in FIG. 10C and FIG. 10D represent polynomial regression analyses, with a goodness-of-fit (R²) of 0.80 and 0.60, respectively. Regression analysis of data in FIGS. 10A-10B and FIGS. 10E-10F did not show statistically significant predictions for comparison (R²<0.60).

FIGS. 11A-11C depict the assessment of PFNA interaction avidity (dispersion) and protein amino acid frequency. Polar amino acids with charged (FIG. 11A) or uncharged (FIG. 11B) side chains are shown. Amino acids designated as nonpolar are shown in FIG. 11C. Dashed lines represent regression analyses, with only those trends achieving a goodness-of-fit (R²)>0.60 shown. R² value for each comparison is shown in the bottom right corner of the plot.

FIG. 12A depicts a workflow diagram of the MedusaDock protein-ligand docking algorithm. FIGS. 12B-12E depicts the relative frequency of hydrogen bonding between PFNA's carboxylic acid (FIG. 12B) and fluorine atoms (FIG. 12C) with each protein's solvent shell water molecules (H₂O), backbone nitrogen and oxygen (NB, OB) and solvent accessible amino acid side chains capable of forming hydrogen bonds. FIG. 12D: relative frequency of PFNA hydrophobic contacts with protein solvent accessible amino acids. FIG. 12E: box and whisker plot of change in Gibbs free energy for PFNA ligands docked in protein binding sites. Individual points represent rotamer lowest binding free energy in a selected binding site; n=7-12 binding sites per protein.

FIG. 13 depicts a model of a PFNA ligand docked with GFP (PDB structure 3UFZ).

FIGS. 14A-14L depict circular dichroism spectra of Hb (FIG. 14A), GFP (FIG. 14B), BSA (FIG. 14C), β-Gal (FIG. 14D), rabbit serum IgG (FIG. 14E), transferrin (FIG. 14F), and urease (FIG. 14G) in the absence (black line) or presence (dashed lines) of PFNA. Increasing dash sequence indicated higher concentration of PFNA. FIG. 14H: Change in molar ellipticity at 216 nm (Δθ216) of PFNA-treated proteins, relative to native controls, as a function of increasing ligand concentration. FIG. 14I: Percentage change in secondary structural motifs of Hb, β-Gal and IgG between the native protein and 1 mM PFNA treated samples. β-sheet structures were further differentiated into parallel and antiparallel conformations. Hb, β-Gal and IgG were prioritized for analysis based on their significant spectral shift in the presence of 1 mM PFNA. FIGS. 14J-14L: Sensograms for BSA (FIG. 14J), β-gal (FIG. 14K), and IgG (FIG. 14L) showing relative response (R.U.) at 10, 25, 50, 75, and 100 μM.

FIG. 15 depicts the melting temperature (Tm) of 5 test proteins dissolved in either PBS (Aq.) or Perfluorooctane (PFOc).

FIGS. 16A-16C depict that the thermal stability of β-gal, GFP, and Trypsin is enhanced when the proteins are dissolved in a PFNA/PFOc mixture, compared to aqueous solutions of the proteins.

FIGS. 17A-17B depict the uptake of GFP in human lung A549 cells after 2 and 24 hours of incubation in the absence/presence of PFNA. Results are shown in relative fluorescence units (RFU).

FIGS. 18A-18B depict the uptake of Cy5-labeled BSA in human lung A549 cells after 2 and 24 hours of incubation in the absence/presence of PFNA. Results are shown in relative fluorescence units (RFU).

FIGS. 19A-19B depicts the uptake of Texas Red-labeled Transferrin in human lung A549 cells after 2 and 24 hours of incubation in the absence/presence of PFNA. Results are shown in relative fluorescence units (RFU).

FIGS. 20A-20D depicts annexin-V/PI co-staining of A549 cells treated without or with PFNA at 2 and 24 hours. FIGS. 20A-20C: Flow cytometry scatter plots. FIG. 20D: quadrant data comparing the population of unaffected cells (native), to cells displaying lipid translocation (Lip. Tran.) membrane permeabilization (Mem. Perm.), or both.

FIG. 21 depicts confocal micrographs of eGFP-PC9 cells in the absence (top) and presence (bottom) of PFNA.

FIG. 22 depicts an analysis of average cellular fluorescence (in relative fluorescence units, RFU) for cells treated without/with PFNA for 1, 2 and 5 hours. Statistical significance was determined using unpaired t-test, with n.s.=not significant, * p<0.05, ** p<0.01 and *** p<0.001.

FIG. 23 depicts analysis of cellular fluorescent area (in relative area units, RAU) for cells treated without/with PFNA for 1, 2 and 5 hours. Statistical significance was determined using unpaired t-test, with n.s.=not significant, * p<0.05, ** p<0.01 and *** p<0.001.

FIGS. 24A-24B show that PFOc samples remain sterile after contamination with E. coli. FIG. 24A: Diagram of contamination experiment. FIG. 24B: Samples in PBS (top) and PFOc (bottom) after contamination with E. coli.

FIG. 25 depicts selected fluorochemical compounds 6 (6), 7 (7), 8 (8), 9 (9), and 10 (10), which were based off Formula II with 6, 7, 8, 9, and 10 repeats (indicated as ‘x’ in Formula II), respectively.

FIG. 26 depicts fluorochemical compounds 21, 22, 23, and 24, which are based on Formula VIII.

FIGS. 27A-27D depict the fluorochemical-mediated dispersion of test proteins bovine serum albumin (BSA, FIG. 27A), β-Galactosidase (β-Gal, FIG. 27B), trypsin (FIG. 27C), and rabbit serum immunoglobulin G (IgG, FIG. 27D) into solutions containing fluorochemical compounds. Results are displayed as percent soluble protein relative to initial loading. The compound ‘0’ indicates a control sample absent of an additive.

FIG. 28 depicts a viability assay for HepG2 cells treated with two first generation compounds and two second generation compounds.

FIG. 29 depicts additional fluorochemical compounds 25, 26, 27, 28, and 29, based on Formula VIII.

FIGS. 30A-30B depict additional fluorochemical compounds, based on Formula IX.

FIGS. 31A-31B depict additional fluorochemical compounds based on Formula X.

FIG. 32 depicts time-dependent enzymatic activity curves of β-Gal in plasma delivered systemically in either saline (β-GalSaline) or extracted PFOc (β-GalExt. PFOc) vehicle.

FIG. 33 depicts box and whisker plot of n=4-5 technical replicates of enzymatic activity of β-Gal in plasma delivered systemically in either saline (β-GalSaline) or extracted PFOc (β-GalExt. PFOc) vehicle.

FIGS. 34A-34B depict representative histopathologic images of lung, kidney, liver, and spleen tissue section from C57BL/6J mice 24 hours after administration of saline, β-GalSaline or β-GalExt. PFOc. FIG. 34A: Each imaging group consisted of n=4 mice, with 4 random fields per section collected at 10× magnification in a blinded manner; scale bar=100 μm. FIG. 34B: Same data as 34A, collected at 40× magnification.

FIG. 35 depicts a structural library of protein dispersants (PD).

FIGS. 36A-36D depict dispersion efficiency of (FIG. 36A) bovine serum albumin (BSA), (FIG. 36B) (β-Galactosidase (β-Gal), (FIG. 36C) rabbit serum immunoglobulin G (IgG) and (FIG. 36D) trypsin in PFOc using the indicated dispersant (PD:protein molar ratio of 1000:1). Results displayed as percent soluble protein relative to initial loading. PD-7 is highlighted via cross-hatching to aid indexing results to dispersant structure. Data shown in panels represents the average±s.d. of n=3 technical replicates.

FIGS. 37A-37B depict influence of dispersant (FIG. 37A) partition coefficient (log P) and (FIG. 37B) dissociation coefficient (pKa) on protein dispersion efficiency. Data shown in panels represents the average±s.d. of n=3 technical replicates.

FIGS. 38A-38B depict cytotoxicity curves and corresponding results for the indicated protein dispersant towards HepG2 human hepatic carcinoma cells. FIG. 38B: Indicates the concentration at which 50% inhibition of cell (IC50) occurs.

FIGS. 39A-39B depict temperature-dependent CD spectra of the indicated test protein solubilized in PFOc using the PD-7 dispersant, or PBS as a control.

FIG. 40 depicts the change in minimum CD ellipticity over the 40 °C-85 °C temperature interval for the indicated protein dissolved in either PBS or PFOc.

FIGS. 41A-41D depict representative transmission electron micrograph of (FIG. 41A) BSA, (FIG. 41B) PD-7 in PFOc, (FIG. 41C) PD-7 coated BSA assemblies in PFOc, and (FIG. 41D) PD-7 coated BSA fibrils in PFOc solvent. Scale bar in FIGS. 41A-41B represent 2 μm and 500 nm, respectively. FIG. 41C: Scale bar=2 μm. FIG. 41D: Scale bar=100 nm.

FIG. 42 depicts superimposed ¹H NMR region demonstrating the downfield shift of PD-7's —COOH protein in the absence and presence of BSA.

FIG. 43 depicts a superimposed ¹H NMR spectra of PD-7 (60 mM) and PD-7:BSA (1000:1 molar ratio) complex. Inset shows magnified region of PD-7 —COOH proton chemical shift (Δδ).

FIG. 44 depicts a superimposed ¹H NMR spectra of PD-2 (60 mM) and PD-2:BSA (1000:1 molar ratio) complex. Inset shows magnified region of PD-2 —COOH proton chemical shift (Δδ).

FIG. 45 depicts the change in ¹H (—COOH) and ¹⁹F (—CF₃) NMR chemical shift (Δδ) for PD-2 (grey) and PD-7 (black) following coordination with the BSA protein.

FIGS. 46A-46B depict superimposed ¹⁹F NMR spectra. FIG. 46A: PD-7 (60 mM) and PD-7:BSA (1000:1 molar ratio) complex; inset shows magnified region of PD-7 —CFₓ proton chemical shift (Δδ). FIG. 47B: PD-2 (60 mM) and PD-2:BSA (1000:1 molar ratio) complex. Inset shows magnified region of PD-2 —CFₓ proton chemical shift (Δδ).

FIG. 47 depicts stacked FTIR spectra of PD-7, BSA and the PD-7:BSA complex after elution into PBS.

FIGS. 48A-48D depict residue-specific hydrogen-bonding interactions of PD-2 (grey) and PD-7 (black) with (FIG. 48A) Hb, (FIG. 48B) β-Gal, (FIG. 48C) GFP and (FIG. 48D) trypsin. Data reported as the percentage of total hydrogen bonds divided by the frequency normalized residue count in the protein.

FIG. 49 depicts the molecular organization of PD-2:BSA (top panels) and PD-7:BSA (lower panels) assemblies at the initial (t=0 ns) and 10 ns simulation time points.

FIGS. 50A-50B depict the molecular model of (FIG. 50A) PD-2 and (FIG. 50B) PD-7 docked to the surface of BSA, with the indicated distance of representative hydrogen bonds.

FIG. 51 depicts residue-specific hydrogen-bonding interactions of PD-2 (grey) and PD-7 (black) with BSA. Data reported as the percentage of total hydrogen bonds divided by the frequency normalized residue count in the protein.

FIGS. 52A-52D depict the change in number of hydrogen bonds and free energy after interaction with the protein surface, as well as free energy of dispersant oligomerization, for PD-2 (grey) and PD-7 (black) over the 10 ns simulation time. FIGS. 52C-52D: Free energy plots for dispersant interactions with Hb (a), β-Gal (b), GFP (c) and trypsin (d).

FIG. 53 depicts representative photographs of agar plates streaked with BSA formulations in PBS (top panel) or PFOc (lower panel) contaminated with the indicated pathogen. MRSA=methicillin resistant Staphylococcus aureus.

FIGS. 54A-54C depict the relative activity of the β-Gal protein dispersed in PBS or PFOc solvents without and with contamination by (FIG. 54A) proteinase K, (FIG. 54B) bleach or (FIG. 54C) hydrochloric acid. Data shown as average±s.d. of n=3 technical replicates. Statistical significance between conditions is indicated by a line using unpaired Student's t-test with n.s.=not significant, * p<0.05, ** p<0.01, and *** p<0.001.

FIG. 55 depicts time-dependent enzymatic activity of β-Gal in plasma delivered systemically in either saline (β-GalSaline) or extracted PFOc (β-GalExt. PFOc). Data shown as average±s.d. of n=5 technical replicates. Statistical significance determined using Student's t-test and represented as n.s.=not significant, * p<0.05.

FIG. 56 depicts serologic toxicology results from C57BL/6J mice 24 hours after administration of saline (control), β-GalSaline or β-GalExt. PFOc. Data shown as box and whisker plot±s.d. of n=5 technical replicates. Statistical significance determined using Student's t-test and represented as * p<0.05; all other comparisons were found not to be significant (p>0.05).

### DETAILED DESCRIPTION

Unless defined otherwise, all technical and scientific terms have the same meaning as commonly understood by one of ordinary skill in the art to which the embodiments disclosed belongs.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any step or composition that uses the transitional phrase of “comprise” or “comprising” can also be said to describe the same with the transitional phase of “consisting of” or “consists.”

The term “perfluorinated compound” refers to an organofluorine compound that contains only carbon-fluorine and carbon-carbon bonds and may or may not also contain heteroatoms. Non-limiting examples of perfluorinated compounds include, but are not limited to, perfluoroalkyl substances. The term “perfluoroalkyl substance,” abbreviated as “PFAS,” refers to organofluorine compounds that possess C—F bonds and other heteroatom functional groups (e.g. —OH, —CO₂H).

In some embodiments, a composition is provided, the composition comprising a fluorinated solvent, a dispersant compound, and a protein of interest, wherein: the dispersant compound interacts with the protein of interest; and the protein of interest is dissolved in the fluorinated solvent.

In some embodiments, the fluorinated solvent is selected from the group consisting of perfluoropentane, perfluorohexane, perfluoroheptane, perfluorooctane, perfluoromethylcyclohexane, perfluoro-1,3-dimethylcyclohexane, and perfluorodecalin.

In some embodiments, the fluorinated solvent compound comprises Formula I:

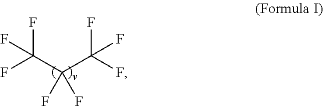

wherein the formula is (CF₂)v(CF₃)₂ and v is 3 (perfluoropentane), 4 (perfluorohexane), 5 (perfluoroheptane), or 6 (perfluorooctane). In some embodiments, the fluorinated solvent is perfluorohexane. In some embodiments, the fluorinated solvent is perfluorooctane.

In some embodiments, the solvent compound comprises Compound 0. In some embodiments, Compound 0 comprises perfluorohexane (PFH). In some embodiments, Compound 0 comprises the formula C₆F₁₄. In some embodiments, the solvent compound comprises Formula I ((CF₂)v(CF₃)₂), wherein v is 4 ((CF₂)₄(CF₃)₂), and the compound is Compound 0 comprising perfluorohexane. In some embodiments, perfluorohexane is 1,1,1,2,2,3,3,4,4,5,5,6,6,6-tetradecafluorohexane. In some embodiments, the molecular weight of Compound 0 is about 338 g/mol, or about the molecular weight of PFH.

In some embodiments, the fluorinated solvent comprises a solvent chosen from Table 1.

TABLE 1
Perfluorinated solvents

| Formula | Compound name | Abbreviation |
|---|---|---|
| C₅F₁₂ | perfluoropentane | PFP |
| C₆F₁₄ | perfluorohexane | PFH |
| C₇F₁₆ | perfluoroheptane | |
| C₈F₁₈ | perfluorooctane | PFO |
| C₇F₁₄ | perfluoromethylcyclohexane | |
| C₈F₁₆ | perfluoro-1,3-dimethylcyclohexane | |
| C₁₀F₁₈ | perfluorodecalin | |

In some embodiments, the dispersant compound is perfluorinated. In some embodiments, a composition is provided, the composition comprising a fluorinated solvent, a perfluorinated dispersant compound, and a protein of interest, wherein: the perfluorinated dispersant compound interacts with the protein of interest; and the protein of interest is dissolved in the fluorinated solvent. In some embodiments, the dispersant compound interacts with the protein of interest via hydrogen bonding with nitrogen donors present in the protein backbone and amino acid side chains of the protein of interest. In some embodiments, the protein of interest comprises a non-native secondary structure. In some embodiments, the dispersant compound is perfluorinated.

In some embodiments, a composition is provided, the composition comprising a fluorinated solvent, a perfluorinated dispersant compound, and a protein of interest, wherein: the perfluorinated dispersant compound interacts with the protein of interest; the protein of interest is dissolved in the fluorinated solvent; the fluorinated solvent is chosen from perfluoropentane, perfluorohexane, perfluoroheptane, perfluorooctane, perfluoromethylcyclohexane, perfluoro-1,3-dimethylcyclohexane, or perfluorodecalin; and the perfluorinated dispersant compound is chosen from a compound that comprises: Formula I, Formula II, Formula III, Formula IV, Formula V, Formula VI, Formula VII, or Formula VIII. In some embodiments, a composition is provided, the composition comprising a fluorinated solvent, a perfluorinated dispersant compound, and a protein of interest, wherein: the perfluorinated dispersant compound interacts with the protein of interest; the protein of interest is dissolved in the fluorinated solvent; the fluorinated solvent is chosen from perfluoropentane, perfluorohexane, perfluoroheptane, perfluorooctane, perfluoromethylcyclohexane, perfluoro-1,3-dimethylcyclohexane, or perfluorodecalin; and the perfluorinated dispersant compound is chosen from: perfluorohexane (Compound 0), perfluorooctane (Compound 1), perfluorotetradecane (Compound 2), trifluoroacetic acid (Compound 3), perfluoropropionic acid (Compound 4), perfluoropentanoic acid (Compound 5), perfluorooctanoic acid (Compound 6), perfluoronanonic acid (Compound 7), perfluorodecanoic acid (Compound 8), perfluoroundecanoic acid (Compound 9), perfluorododecanoic acid (Compound 10), perfluorotetradecanoic acid (Compound 11), perfluorooctadecanoic acid (Compound 12), perfluorocyclohexanecarboxylic acid (Compound 13), (2S) 2-FMOC-amino-3-pentafluorophenyl propanoic acid (Compound 14), pentafluorophenol (Compound 15), pentafluorobenzaldehyde (Compound 16), perfluorooctanesulfonic acid (Compound 17), 1H,1H-heptafluorobutylamine (Compound 18), perfluorooctylamine (Compound 19), and 1H,1H-perfluorononylamine (Compound 20). In some embodiments, the dispersant compound is perfluorinated. In some embodiments, the dispersant compound is a perfluorinated compound chosen from the group that includes: perfluorooctanoic acid (Compound 6), perfluoronanonic acid (Compound 7), perfluorodecanoic acid (Compound 8) perfluoroundecanoic acid (Compound 9), perfluorododecanoic acid (Compound 10), 2,2-(1,1,1,2,2,3,3,4,4,5,5,6,6-tridecafluorooctane)-2-cyanoacetic acid (Compound 21), 2,2-(1,1,1,2,2,3,3,4,4,5,5,6,6-tridecafluorooctane)-2-acetic acid (Compound 22), 2,2-(1,1,1,2,2,3,3,4,4,5,5,6,6,7,7,8,8-heptadecafluorodecane)-2-cyanoacetic acid (Compound 23), 2,2-(1,1,1,2,2,3,3,4,4,5,5,6,6,7,7,8,8-heptadecafluorodecane)-acetic acid (Compound 24), 2,2-(1,1,1,2,2,3,3,4,4-nonafluorohexane)-2-cyanoacetic acid (Compound 25), 2,2-(1,1,1,2,2,3,3,4,4-nonafluorohexane)-2-acetic acid (Compound 26), 2,2-(1,1,1,2,2,3,3,4,4-nonafluorohexane)-2-diacetic acid (Compound 27), 2,2-(1,1,1,2,2,3,3,4,4,5,5,6,6-tridecafluorooctane)-2-diacetic acid (Compound 28), or 2,2-(1,1,1,2,2,3,3,4,4,5,5,6,6,7,7,8,8-heptadecafluorodecane)-2-diacetic acid (Compound 29), as described herein.

In some embodiments, the dispersant compound is a perfluorinated compound chosen from the group that includes: perfluorohexane (Compound 0), perfluorooctane (Compound 1), perfluorotetradecane (Compound 2), trifluoroacetic acid (Compound 3), perfluoropropionic acid (Compound 4), perfluoropentanoic acid (Compound 5), perfluorooctanoic acid (Compound 6), perfluoronanonic acid (Compound 7), perfluorodecanoic acid (Compound 8), perfluoroundecanoic acid (Compound 9), perfluorododecanoic acid (Compound 10), perfluorotetradecanoic acid (Compound 11), perfluorooctadecanoic acid (Compound 12), perfluorocyclohexanecarboxylic acid (Compound 13), (2S) 2-FMOC-amino-3-pentafluorophenyl propanoic acid (Compound 14), pentafluorophenol (Compound 15), pentafluorobenzaldehyde (Compound 16), perfluorooctanesulfonic acid (Compound 17), 1H,1H-heptafluorobutylamine (Compound 18), perfluorooctylamine (Compound 19), and 1H,1H-perfluorononylamine (Compound 20).

In some embodiments, the dispersant compound is perfluorinated. In some embodiments, the dispersant compound is a perfluorinated compound chosen from the group that includes: perfluorooctanoic acid (Compound 6), perfluoronanonic acid (Compound 7), perfluorodecanoic acid (Compound 8) perfluoroundecanoic acid (Compound 9), perfluorododecanoic acid (Compound 10), 2,2-(1,1,1,2,2,3,3,4,4,5,5,6,6-tridecafluorooctane)-2-cyanoacetic acid (Compound 21), 2,2-(1,1,1,2,2,3,3,4,4,5,5,6,6-tridecafluorooctane)-2-acetic acid (Compound 22), 2,2-(1,1,1,2,2,3,3,4,4,5,5,6,6,7,7,8,8-heptadecafluorodecane)-2-cyanoacetic acid (Compound 23), 2,2-(1,1,1,2,2,3,3,4,4,5,5,6,6,7,7,8,8-heptadecafluorodecane)-acetic acid (Compound 24), 2,2-(1,1,1,2,2,3,3,4,4-nonafluorohexane)-2-cyanoacetic acid (Compound 25), 2,2-(1,1,1,2,2,3,3,4,4-nonafluorohexane)-2-acetic acid (Compound 26), 2,2-(1,1,1,2,2,3,3,4,4-nonafluorohexane)-2-diacetic acid (Compound 27), 2,2-(1,1,1,2,2,3,3,4,4,5,5,6,6-tridecafluorooctane)-2-diacetic acid (Compound 28), or 2,2-(1,1,1,2,2,3,3,4,4,5,5,6,6,7,7,8,8-heptadecafluorodecane)-2-diacetic acid (Compound 29), as described herein.

In some embodiments, the dispersant compound is a perfluorinated compound chosen from the group that includes: perfluorooctane (Compound 1), perfluorotetradecane (Compound 2), trifluoroacetic acid (Compound 3), perfluoropropionic acid (Compound 4), perfluoropentanoic acid (Compound 5), perfluorooctanoic acid (Compound 6/Compound 6), perfluoronanonic acid (Compound 7/Compound 7), perfluorodecanoic acid (Compound 8/Compound 8), perfluoroundecanoic acid (Compound 9/Compound 9), perfluorododecanoic acid (Compound 10/Compound 10), perfluorotetradecanoic acid (Compound 11), perfluorooctadecanoic acid (Compound 12), perfluorocyclohexanecarboxylic acid (Compound 13), (2S) 2-FMOC-amino-3-pentafluorophenyl propanoic acid (Compound 14), pentafluorophenol (Compound 15), pentafluorobenzaldehyde (Compound 16), perfluorooctanesulfonic acid (Compound 17), 1H,1H-heptafluorobutylamine (Compound 18), perfluorooctylamine (Compound 19), 1H,1H-perfluorononylamine (Compound 20), 2,2-(1,1,1,2,2,3,3,4,4,5,5,6,6-tridecafluorooctane)-2-cyanoacetic acid (Compound 21), 2,2-(1,1,1,2,2,3,3,4,4,5,5,6,6-tridecafluorooctane)-2-acetic acid (Compound 22), 2,2-(1,1,1,2,2,3,3,4,4,5,5,6,6,7,7,8,8-heptadecafluorodecane)-2-cyanoacetic acid (Compound 23), 2,2-(1,1,1,2,2,3,3,4,4,5,5,6,6,7,7,8,8-heptadecafluorodecane)-acetic acid (Compound 24), 2,2-(1,1,1,2,2,3,3,4,4-nonafluorohexane)-2-cyanoacetic acid (Compound 25), 2,2-(1,1,1,2,2,3,3,4,4-nonafluorohexane)-2-acetic acid (Compound 26), 2,2-(1,1,1,2,2,3,3,4,4-nonafluorohexane)-2-diacetic acid (Compound 27), 2,2-(1,1,1,2,2,3,3,4,4,5,5,6,6-tridecafluorooctane)-2-diacetic acid (Compound 28), or 2,2-(1,1,1,2,2,3,3,4,4,5,5,6,6,7,7,8,8-heptadecafluorodecane)-2-diacetic acid (Compound 29), as described herein.

In some embodiments, the dispersant compound is a perfluorinated compound chosen from the group that includes any compound described in any one of Tables 2-6.

In some embodiments, the dispersant compound is a perfluorinated compound with a chemical formula chosen from a group that is described in Table 2. In some embodiments, the chemical formula of the perfluorinated dispersant compound is based off of Formula I, Formula II, Formula III, Formula IV, Formula V, Formula VI, Formula VII, or Formula VII. In some embodiments, the perfluorinated dispersant compound comprises the SMILES ID chosen from the group as described in Table 6.

In some embodiments, the perfluorinated dispersant compound comprises a chemical name as chosen from the group that is described in Table 5. In some embodiments, the perfluorinated dispersant compound can be abbreviated as described in Table 5. In some embodiments, the perfluorinated dispersant compound comprises a chemical group R1 chosen from the group that is described in Table 4. In some embodiments, the perfluorinated dispersant compound comprises a chemical group R1_Extended chosen from the group that is described in Table 4. In some embodiments, the perfluorinated dispersant compound comprises a chemical name chosen from the group described in Table 3, where R1_Extended comprises the chemical group R1_Extended chosen from the group described in Table 4.

In some embodiments, the fluorinated solvent is chosen from the group described in Table 1 and the perfluorinated dispersant compound is chosen from the group of compounds described in any one of Tables 2-6.

TABLE 2
Dispersant Compound

| Formula | | | | | |
|---|---|---|---|---|---|
| ID | Chemical Formula | | ID | Chemical Formula | |
| I | (CF₂)ᵥ(CF₃)₂ | V = 4 | 0 | C₆F₁₄ | (CF₂)₄(CF₃)₂ |
| | | V = 6 | 1 | C₈F₁₈ | (CF₂)₆(CF₃)₂ |
| | | V = 12 | 2 | C₁₄F₃₀ | (CF₂)₁₂(CF₃)₂ |
| II | (CF₂)ₓCF₃COOH | X = 0 | 3 | C₂HF₃O₂ | (CF₂)₀CF₃COOH |
| | | X = 1 | 4 | C₃HF₅O₂ | (CF₂)₁CF₃COOH |
| | | X = 3 | 5 | C₅HF₉O₂ | (CF₂)₃CF₃COOH |
| | | X = 6 | 6 | C₈HF₁₅O₂ | (CF₂)₆CF₃COOH |
| | | X = 7 | 7 | C₉HF₁₇O₂ | (CF₂)₇CF₃COOH |
| | | X = 8 | 8 | C₁₀HF₁₉O₂ | (CF₂)₈CF₃COOH |
| | | X = 9 | 9 | C₁₁HF₂₁O₂ | (CF₂)₉CF₃COOH |
| | | X= 10 | 10 | C₁₂HF₂₃O₂ | (CF₂)₁₀CF₃COOH |
| | | X = 12 | 11 | C₁₃HF₂₇O₂ | (CF₂)₁₂CF₃COOH |
| | | X = 16 | 12 | C₁₈HF₃₇O₂ | (CF₂)₁₆CF₃COOH |
| III | C₆F₁₁COOH | | 13 | C₇HF₁₁O₂ | |
| IV | C₂₄H₁₆F₅NO₄ | | 14 | C₂₄H₁₆F₅NO₄ | |
| V | C₆F₅R | R = OH | 15 | C₆HF₅O | C₆F₅R |
| | | R = COH | 16 | C₇HF₅O | C₆F₅R |
| VI | (CF₂)_{Y}CF₃SO₂OH | Y = 7 | 17 | C₈HF₁₇O₃S | (CF₂)₇CF₃SO₂OH |
| VII | NH₂CH₂(CF₂)_{z}CF₃ | Z = 2 | 18 | C₄H₄F₇N | NH₂CH₂(CF₂)₂CF₃ |
| | | Z = 6 | 19 | C₈H₄F₁₅N | NH₂CH₂(CF₂)₆CF₃ |
| | | Z = 7 | 20 | C₉H₄F₁₇N | NH₂CH₂(CF₂)₇CF₃ |
| VIII | C₅H₈(R1)₂(R2) | R1 = C₆F₁₃ | 21 | C₁₉H₉F₂₆ | C₅H₈(C₆F₁₃)₂₍CN) |
| | COOH | R2 = CN | | NO₂ | COOH |
| | | R1= C₆F₁₃ | 22 | C₁₈H₁₀F₂₆O₂ | C₅H₈(C₆F₁₃)₂(H) |
| | | R2 = H | | | COOH |
| | | R1 = C₈F₁₇ | 23 | C23H₉F₃₄ | C₅H₈(C₈F₁₇)₂ |
| | | R2 = CN | | NO₂ | (CN)COOH |
| | | R1 = C₈F₁₇ | 24 | C₂₂H₁₀F₃₄O₂ | C₅H₈(C₈F₁₇)₂(H) |
| | | R2 = H | | | COOH |
| | | R1 = C₄F₉ | 25 | C₁₅H₉F₁₈NO₂ | C₅H₈(C₄F₉)₂(CN) |
| | | R2 = CN | | | COOH |
| | | R1 = C₄F₉ | 26 | C₁₄H₁₀F₁₈O₂ | C₅H₈(C₄F₉)₂(H) |
| | | R2 = H | | | COOH |
| | | R1 = C₄F₉; | 27 | C₁₅H₁₀F₁₈O₄ | C₅H₈(C₄F₉)₂ |
| | | R2 = COOH | | | (COOH)₂ |
| | | R1 = C₆F₁₃; | 28 | C₁₉H₁₀F₂₆O₄ | C₅H₈(C₆F₁₃)₂ |
| | | R = COOH | | | (COOH)₂ |
| | | R1 = C₈F₁₇; | 29 | C₂₃H₁₀F₃₄O₄ | C₅H₈(C₈F₁₇)₂ |
| | | R2 = COOH | | | (COOH)₂ |

| | | | | |
|---|---|---|---|---|
| IX | C₃NHO₂(R1)(R2) | R1 = COOH | 30 | C₃NHO₂(COOH)(C₇H₁₅N₂O) |
| | (R3)(R4) | R2 = C₇H₁₅N₂O | | (C₈F₁₇)(H) |
| | | R3 = C₈F₁₇ | | |
| | | R4 = H | | |
| X | C₈NH₁₀O₂ | R1 = CN, H, | 31 | C₈NH₁₀O₂(COOH)₂ |
| | (R1)₂(R2) | COOH | | (C₇H₁₅N₂O)(C₆F₁₃)H |
| | (R3)(R4) | R2 = C₇H₁₅N₂O | | |
| | | R3 = C₆F₁₃ | | |
| | | R4 = H | | |

TABLE 3

| | | Associated |
|---|---|---|
| Formula | Formula name | Compounds |
| C₅H₈(R1)₂(CN) | 2,2-(R1_Extended)- | 21, 23, 25 |
| COOH | | |
| C(R1_Extended)₂ | 2-cyanoacetic acid | |
| (CN)COOH | | |
| C₅H₈(R1)₂(H) | 2,2-(R1_Extended)- | 22, 24, 26 |
| COOH | | |
| C(R1_Extended)₂ | 2-acetic acid | |
| (H)COOH | | |
| C₅H₈(R1)₂(COOH) | 2,2-(R1_Extended)- | 27, 28, 29 |
| COOH | | |
| C(R1_Extended)₂ | 2-diacetic acid | |
| (COOH)COOH | | |

TABLE 4

| | R1_ | R1_Extended | Associated |
|---|---|---|---|
| R1 | Extended | Group name | Compounds |
| C₄F₉ | C₆H₅F₉ | 1,1,1,2,2,3,3,4,4- | 25, 26, 27 |
| | | nonafluorohexane | |
| C₆F₁₃ | C₈H₅F₁₃ | 1,1,1,2,2,3,3,4,4,5,5,6,6- | 21, 22, 28 |
| | | tridecafluorooctane | |
| C₈F₁₇ | C₁₀H₅F₁₇ | 1,1,1,2,2,3,3,4,4,5,5,6,6, | 23, 24, 29 |
| | | 7,7,8,8-heptadecafluoro- | |
| | | decane | |

TABLE 5

| Dispersant | | |
|---|---|---|
| Compound | | Abbrevi- |
| ID | Compound name | ation |
| 0 | perfluorohexane | PFH |
| 1 | perfluorooctane | PFO |
| 2 | perfluorotetradecane | |
| 3 | trifluoroacetic acid | TFA |
| 4 | perfluoropropionic acid | PFPrA |
| 5 | perfluoropentanoic acid | PFPeA |
| 6 | perfluorooctanoic acid | PFOA |
| 7 | perfluoronanonic acid | PFNA |
| 8 | perfluorodecanoic acid | PFDA |
| 9 | perfluoroundecanoic acid | PFUnDA |
| 10 | perfluorododecanoic acid | |
| 11 | perfluorotetradecanoic acid | PFTeDA |
| 12 | perfluorooctadecanoic acid | |
| 13 | perfluorocyclohexanecarboxylic acid | |
| 14 | (2S) 2-FMOC-amino-3- | |
| | pentafluorophenyl propanoic acid | |
| 15 | pentafluorophenol | |
| 16 | pentafluorobenzaldehyde | |
| 17 | perfluorooctanesulfonic acid | PFOS |
| 18 | 1H,1H-heptafluorobutylamine | |
| 19 | 1H,1H-pentadecafluorooctylamine | |
| 20 | 1H,1H-perfluorononylamine | |
| 21 | 2,2-(1,1,1,2,2,3,3,4,4,5,5,6,6- | |
| | tridecafluorooctane)-2-cyanoacetic acid | |
| 22 | 2,2-(1,1,1,2,2,3,3,4,4,5,5,6,6-trideca | |
| | fluorooctane)-2-acetic acid | |
| 23 | 2,2-(1,1,1,2,2,3,3,4,4,5,5,6,6,7,7,8,8- | |
| | heptadecafluorodecane)-2-cyanoacetic acid | |
| 24 | 2,2-(1,1,1,2,2,3,3,4,4,5,5,6,6,7,7,8,8- | |
| | heptadecafluorodecane)-acetic acid | |
| 25 | 2,2-(1,1,1,2,2,3,3,4,4-nonafluorohexane)-2- | |
| | cyanoacetic acid | |
| 26 | 2,2-(1,1,1,2,2,3,3,4,4-nonafluorohexane)-2- | |
| | acetic acid | |
| 27 | 2,2-(1,1,1,2,2,3,3,4,4-nonafluorohexane)-2- | |
| | diacetic acid | |
| 28 | 2,2-(1,1,1,2,2,3,3,4,4,5,5,6,6- | |
| | tridecafluorooctane)-2-diacetic acid | |
| 29 | 2,2-(1,1,1,2,2,3,3,4,4,5,5,6,6,7,7,8,8- | |
| | heptadecafluorodecane)-2-diacetic acid | |

TABLE 6
Dispersant Compound

| ID | SMILES ID |
|---|---|
| 0 | C(C(C(C(F)(F)F)(F)F)(F)F)(C(C(F)(F)F)(F)F)(F)F |
| 1 | C(C(C(C(C(F)(F)F)(F)F)(F)F)(F)F)(C(C(C(F)(F)F)(F)F)(F)F)(F)F |
| 2 | C(C(C(C(C(C(C(C(F)(F)F)(F)F)(F)F)(F)F)(F)F)(F)F)(F)F)(C(C(C(C(C(C(F)(F)F)(F)F) |
| | (F)F)(F)F)(F)F)(F)F)(F)F |
| 3 | C(═O)(C(F)(F)F)O |
| 4 | C(═O)(C(C(F)(F)F)(F)F)O |
| 5 | C(═O)(C(C(C(C(F)(F)F)(F)F)(F)F)(F)F)O |
| 6 | C(═O)(C(C(C(C(C(C(C(F)(F)F)(F)F)(F)F)(F)F)(F)F)(F)F)(F)F)O |
| 7 | C(═O)(C(C(C(C(C(C(C(C(F)(F)F)(F)F)(F)F)(F)F)(F)F)(F)F)(F)F)(F)F)O |
| 8 | C(═O)(C(C(C(C(C(C(C(C(C(F)(F)F)(F)F)(F)F)(F)F)(F)F)(F)F)(F)F)(F)F)(F)F)O |
| 9 | C(═O)(C(C(C(C(C(C(C(C(C(C(F)(F)F)(F)F)(F)F)(F)F)(F)F)(F)F)(F)F)(F)F)(F)F)(F)F) |
| | O |
| 10 | C(═O)(C(C(C(C(C(C(C(C(C(C(C(F)(F)F)(F)F)(F)F)(F)F)(F)F)(F)F)(F)F)(F)F)(F)F)(F) |
| | F)(F)F)O |
| 11 | C(═O)(C(C(C(C(C(C(C(C(C(C(C(C(C(F)(F)F)(F)F)(F)F)(F)F)(F)F)(F)F)(F)F)(F)F)(F) |
| | F)(F)F)(F)F)(F)F)(F)F)O |
| 12 | C(═O)(C(C(C(C(C(C(C(C(C(C(C(C(C(C(C(C(C(F)(F)F)(F)F)(F)F)(F)F)(F)F)(F)F)(F) |
| | F)(F)F)(F)F)(F)F)(F)F)(F)F)(F)F)(F)F)(F)F)(F)F)(F)F)O |
| 13 | C(═O)(C1(C(C(C(C(C1(F)F)(F)F)(F)F)(F)F)(F)F)F)O |
| 14 | C1═CC═C2C(═C1)C(C3═CC═CC═C32)COC(═O)N[C@@H](CC4═C(C(═C(C(═C4F) |
| | F)F)F)F)C(═O)O |
| 15 | C1(═C(C(═C(C(═C1F)F)F)F)F)O |
| 16 | C(═O)C1═C(C(═C(C(═C1F)F)F)F)F |
| 17 | C(C(C(C(C(F)(F)S(═O)(═O)O)(F)F)(F)F)(F)F)(C(C(C(F)(F)F)(F)F)(F)F)(F)F |
| 18 | C(C(C(C(F)(F)F)(F)F)(F)F)N |
| 19 | C(C(C(C(C(C(C(C(F)(F)F)(F)F)(F)F)(F)F)(F)F)(F)F)(F)F)N |
| 20 | C(C(C(C(C(C(C(C(C(F)(F)F)(F)F)(F)F)(F)F)(F)F)(F)F)(F)F)(F)F)N |
| 21 | OC(═O)C(CCC(F)(F)C(F)(F)C(F)(F)C(F)(F)C(F)(F)C(F)(F)F)(CCC(F)(F)C(F)(F)C(F) |
| | (F)C(F)(F)C(F)(F)C(F)(F)F)C#N |
| 22 | OC(═O)C(CCC(F)(F)C(F)(F)C(F)(F)C(F)(F)C(F)(F)C(F)(F)F)CCC(F)(F)C(F)(F)C(F) |
| | (F)C(F)(F)C(F)(F)C(F)(F)F |
| 23 | OC(═O)C(CCC(F)(F)C(F)(F)C(F)(F)C(F)(F)C(F)(F)C(F)(F)C(F)(F)C(F)(F)F)(CCC(F) |
| | (F)C(F)(F)C(F)(F)C(F)(F)C(F)(F)C(F)(F)C(F)(F)C(F)(F)F)C#N |
| 24 | OC(═O)C(CCC(F)(F)C(F)(F)C(F)(F)C(F)(F)C(F)(F)C(F)(F)C(F)(F)C(F)(F)F)CCC(F) |
| | (F)C(F)(F)C(F)(F)C(F)(F)C(F)(F)C(F)(F)C(F)(F)C(F)(F)F |
| 25 | OC(═O)C(CCC(F)(F)C(F)(F)C(F)(F)C(F)(F)F)(CCC(F)(F)C(F)(F)C(F)(F)C(F)(F)F)C |
| | #N |
| 26 | OC(═O)C(CCC(F)(F)C(F)(F)C(F)(F)C(F)(F)F)CCC(F)(F)C(F)(F)C(F)(F)C(F)(F)F |
| 27 | OC(═O)C(CCC(F)(F)C(F)(F)C(F)(F)C(F)(F)F)(CCC(F)(F)C(F)(F)C(F)(F)C(F)(F)F)C |
| | (O)═O |
| 28 | OC(═O)C(CCC(F)(F)C(F)(F)C(F)(F)C(F)(F)C(F)(F)C(F)(F)F)(CCC(F)(F)C(F)(F)C(F) |
| | (F)C(F)(F)C(F)(F)C(F)(F)F)C(O)═O |
| 29 | OC(═O)C(CCC(F)(F)C(F)(F)C(F)(F)C(F)(F)C(F)(F)C(F)(F)C(F)(F)C(F)(F)F)(CCC(F) |
| | (F)C(F)(F)C(F)(F)C(F)(F)C(F)(F)C(F)(F)C(F)(F)C(F)(F)F)C(O)═O |
| 30 | OC(═O)C[C@H](NC(═O)C(F)(F)C(F)(F)C(F)(F)C(F)(F)C(F)(F)C(F)(F)C(F)(F)C(F) |
| | (F)F)C(═O) |
| | NCCCNICCOCC1 |
| 31 | OC(═O)C[C@H](NC(═O)C(CCC(F)(F)C(F)(F)C(F)(F)C(F)(F)C(F)(F)C(F)(F)F)CCC |
| | (F)(F) |
| | C(F)(F)C(F)(F)C(F)(F)C(F)(F)C(F)(F)F)C(═O)NCCCNICCOCC1 |

In some embodiments, the dispersant compound comprises a compound described in Table 7.

TABLE 7

| Form- | | | Com - |
|---|---|---|---|
| ula | | | pound |
| ID | Formula | | ID |
| IX | C₃NHO₂ | R1 = CN, H, COOH | 30 |
| | (R1)₂(R2) | R2 = C₇H₁₅N₂O | |
| | (R3)(R4) | R3 = C₄F₉, C₆F₁₃, | |
| | | C₈F₁₇ | |
| | | R4 = CN, H, COOH | |
| X | C₈NH₁₀O₂ | R1 = CN, H, COOH | 31 |
| | (R1)(R2) | R2 = C₇H₁₅N₂O | |
| | (R3)(R4) | R3 = C₄F₉, C₆F₁₃, | |
| | | C₈F₁₇ | |
| | | R4 = CN, H, COOH | |

In some embodiments, the fluorinated solvent comprises Compound 0 (perfluorohexane) and the dispersant compound also comprises Compound 0.

In some embodiments, the protein of interest is dispersed in perfluorohexane in the presence of Compound 0. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane at a molar ratio of about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane at a molar ratio of about 1000:1 with the protein of interest. In some embodiments, the protein of interest dose not phase separate when dispersed in perfluorohexane. In some embodiments, the protein of interest phase separates when dispersed in perfluorohexane, wherein the dispersion efficiency is about 0.5%, 1%, 2%, 3%, 4%, 5%, 6%, 7%, 8%, 9%, or about 10%. In some embodiments, the dispersion efficiency is between about 1% and about 10%. In some embodiments, the dispersion efficiency is about 5%. In some embodiments, the dispersion efficiency is less than 5%.

In some embodiments, the fluorinated solvent comprises Compound 0 (perfluorohexane) and the dispersant compound is chosen from the group that includes perfluorooctane (Compound 1), perfluorotetradecane (Compound 2), trifluoroacetic acid (Compound 3), perfluoropropionic acid (Compound 4), perfluoropentanoic acid (Compound 5), perfluorooctanoic acid (Compound 6/Compound 6), perfluoronanonic acid (Compound 7/Compound 7), perfluorodecanoic acid (Compound 8/Compound 8), perfluoroundecanoic acid (Compound 9/Compound 9), perfluorododecanoic acid (Compound 10/Compound 10), perfluorotetradecanoic acid (Compound 11), perfluorooctadecanoic acid (Compound 12), perfluorocyclohexanecarboxylic acid (Compound 13), (2S) 2-FMOC-amino-3-pentafluorophenyl propanoic acid (Compound 14), pentafluorophenol (Compound 15), pentafluorobenzaldehyde (Compound 16), perfluorooctanesulfonic acid (Compound 17), 1H,1H-heptafluorobutylamine (Compound 18), perfluorooctylamine (Compound 19), 1H,1H-perfluorononylamine (Compound 20), 2,2-(1,1,1,2,2,3,3,4,4,5,5,6,6-tridecafluorooctane)-2-cyanoacetic acid (Compound 21), 2,2-(1,1,1,2,2,3,3,4,4,5,5,6,6-tridecafluorooctane)-2-acetic acid (Compound 22), 2,2-(1,1,1,2,2,3,3,4,4,5,5,6,6,7,7,8,8-heptadecafluorodecane)-2-cyanoacetic acid (Compound 23), 2,2-(1,1,1,2,2,3,3,4,4,5,5,6,6,7,7,8,8-heptadecafluorodecane)-acetic acid (Compound 24), 2,2-(1,1,1,2,2,3,3,4,4-nonafluorohexane)-2-cyanoacetic acid (Compound 25), 2,2-(1,1,1,2,2,3,3,4,4-nonafluorohexane)-2-acetic acid (Compound 26), 2,2-(1,1,1,2,2,3,3,4,4-nonafluorohexane)-2-diacetic acid (Compound 27), 2,2-(1,1,1,2,2,3,3,4,4,5,5,6,6-tridecafluorooctane)-2-diacetic acid (Compound 28), or 2,2-(1,1,1,2,2,3,3,4,4,5,5,6,6,7,7,8,8-heptadecafluorodecane)-2-diacetic acid (Compound 29), as described herein.

In some embodiments, the dispersant compound comprises Formula I:

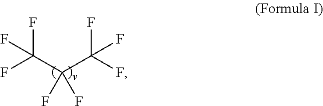

wherein the formula is (CF₂)v(CF₃)₂ and v is 6 or 12. In some embodiments, v is 6 (perfluorooctane). In some embodiments, v is 12 (perfluorotetradecane).

In some embodiments, the dispersant compound comprises Compound 1. In some embodiments, Compound 1 comprises perfluorooctane (PFO). In some embodiments, Compound 1 comprises the formula C₈F₁₈. In some embodiments, the dispersant compound comprises Formula I ((CF₂)_{V}(CF₃)₂), wherein v is 6 ((CF₂)₆(CF₃)₂), and the compound is Compound 1 comprising perfluorooctane. In some embodiments, perfluorooctane is octadecafluorooctane or 1,1,1,2,2,3,3,4,4,5,5,6,6,7,7,8,8,8-octadecafluorooctane. In some embodiments, the molecular weight of Compound 2 is about 438 g/mol, or about the molecular weight of PFO. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 1. In some embodiments, the fluorinated solvent is perfluorohexane and the perfluorinated dispersant compound comprises Compound 1. In some embodiments, the fluorinated solvent is perfluorooctane and the perfluorinated dispersant compound comprises Compound 1 that also comprises perfluorooctane. In some embodiments, the fluorinated solvent is perfluoropentane and the perfluorinated dispersant compound comprises Compound 1. In some embodiments, the fluorinated solvent is perfluoroheptane and the perfluorinated dispersant compound comprises Compound 1. In some embodiments, the fluorinated solvent is perfluoromethylcyclohexane and the perfluorinated dispersant compound comprises Compound 1. In some embodiments, the fluorinated solvent is perfluoro-1-3-dimethylcyclohexane and the perfluorinated dispersant compound comprises Compound 1. In some embodiments, the fluorinated solvent is perfluorodecalin and the perfluorinated dispersant compound comprises Compound 1.

In some embodiments, the protein of interest is dispersed in perfluorohexane in the presence of Compound 1. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 1, wherein the molar ratio of Compound 1 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 1, wherein the molar ratio of Compound 1 is about 1000:1 with the protein of interest. In some embodiments, the protein of interest phase separates when dispersed in perfluorohexane in the presence of Compound 1, but does not phase separate when dispersed in perfluorohexane in the absence of Compound 1. In some embodiments, the protein of interest phase separates when dispersed in perfluorohexane in the presence of Compound 1, wherein the dispersion efficiency is about 1%, 2%, 3%, 4%, 5%, 10%, 15%, 20%, or 25%. In some embodiments, the dispersion efficiency is between about 1% and about 25%. In some embodiments, the dispersion efficiency is about 15%.

In some embodiments, the dispersant compound comprises Compound 2. In some embodiments, Compound 2 comprises perfluorotetradecane. In some embodiments, Compound 2 comprises the formula C₁₄F₃₀. In some embodiments, the dispersant compound comprises Formula I ((CF₂)v(CF₃)₂), wherein v is 12 ((CF₂)₁₂(CF₃)₂), and the compound is Compound 2 comprising perfluorotetradecane. In some embodiments, perfluorotetradecane is 1,1,1,2,2,3,3,4,4,5,5,6,6,7,7,8,8,9,9,10,10,11,11,12,12,13,13,14,14,14-triacontafluorotetradecane. In some embodiments, the molecular weight of Compound 2 is about 738 g/mol, or about the molecular weight of perfluorotetradecane. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 2. In some embodiments, the fluorinated solvent is perfluorohexane and the perfluorinated dispersant compound comprises Compound 2. In some embodiments, the fluorinated solvent is perfluorooctane and the perfluorinated dispersant compound comprises Compound 2. In some embodiments, the fluorinated solvent is perfluoropentane and the perfluorinated dispersant compound comprises Compound 2. In some embodiments, the fluorinated solvent is perfluoroheptane and the perfluorinated dispersant compound comprises Compound 2. In some embodiments, the fluorinated solvent is perfluoromethylcyclohexane and the perfluorinated dispersant compound comprises Compound 2. In some embodiments, the fluorinated solvent is perfluoro-1-3-dimethylcyclohexane and the perfluorinated dispersant compound comprises Compound 2. In some embodiments, the fluorinated solvent is perfluorodecalin and the perfluorinated dispersant compound comprises Compound 2.

In some embodiments, the protein of interest is dispersed in perfluorohexane in the presence of Compound 2. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 2, wherein the molar ratio of Compound 2 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 2, wherein the molar ratio of Compound 2 is about 1000:1 with the protein of interest. In some embodiments, the protein of interest phase separates when dispersed in perfluorohexane in the presence of Compound 2, but does not phase separate when dispersed in perfluorohexane in the absence of Compound 2. In some embodiments, the protein of interest phase separates when dispersed in perfluorohexane in the presence of Compound 2, wherein the dispersion efficiency is about 1%, 2%, 3%, 4%, 5%, 10%, 15%, 20%, or 25%. In some embodiments, the dispersion efficiency is between about 1% and about 25%. In some embodiments, the dispersion efficiency is about 15%.

In some embodiments, the dispersant compound comprises Formula II:

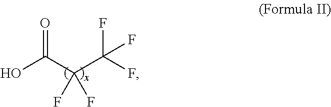

wherein the formula is (CF₂)_{X}(CF₃)COOH and x is 0, 1, 3, 6, 7, 8, 9, 10, 12, or 16. In some embodiments, x is 0 (trifluoroacetic acid). In some embodiments, x is 1 (perfluoropropionic acid). In some embodiments, x is 3 (perfluoropentanoic acid). In some embodiments, x is 6 (perfluorooctanoic acid). In some embodiments, x is 7 (perfluoronanonic acid). In some embodiments, x is 8 (perfluorodecanoic acid). In some embodiments, x is 9 (perfluoroundecanoic acid). In some embodiments, x is 10 (perfluorododecanoic acid). In some embodiments, x is 12 (perfluorotetradecanoic acid). In some embodiments, x is 16 (perfluorooctadecanoic acid).

In some embodiments, the dispersant compound comprises Compound 3. In some embodiments, Compound 3 comprises trifluoroacetic acid (TFA). In some embodiments, Compound 3 comprises the formula C₂HF₃O₂. In some embodiments, the dispersant compound comprises Formula II ((CF₂)_{X}CF₃COOH), wherein x is 0 ((CF₂)₀CF₃COOH), and the compound is Compound 3 comprising trifluoroacetic acid. In some embodiments, trifluoroacetic acid is 2,2,2-trifluoroacetic acid. In some embodiments, the molecular weight of Compound 3 is about 114 g/mol, or about the molecular weight of TFA. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 3. In some embodiments, the fluorinated solvent is perfluorohexane and the perfluorinated dispersant compound comprises Compound 3. In some embodiments, the fluorinated solvent is perfluorooctane and the perfluorinated dispersant compound comprises Compound 3. In some embodiments, the fluorinated solvent is perfluoropentane and the perfluorinated dispersant compound comprises Compound 3. In some embodiments, the fluorinated solvent is perfluoroheptane and the perfluorinated dispersant compound comprises Compound 3. In some embodiments, the fluorinated solvent is perfluoromethylcyclohexane and the perfluorinated dispersant compound comprises Compound 3. In some embodiments, the fluorinated solvent is perfluoro-1-3-dimethylcyclohexane and the perfluorinated dispersant compound comprises Compound 3. In some embodiments, the fluorinated solvent is perfluorodecalin and the perfluorinated dispersant compound comprises Compound 3.

In some embodiments, the protein of interest is dispersed in perfluorohexane in the presence of Compound 3. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 3, wherein the molar ratio of Compound 3 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 3, wherein the molar ratio of Compound 3 is about 1000:1 with the protein of interest. In some embodiments, the protein of interest phase separates when dispersed in perfluorohexane in the presence of Compound 3, but does not phase separate when dispersed in perfluorohexane in the absence of Compound 3. In some embodiments, the protein of interest phase separates when dispersed in perfluorohexane in the presence of Compound 3, wherein the dispersion efficiency is about 1%, 2%, 3%, 4%, 5%, 10%, 15%, 20%, or 25%. In some embodiments, the dispersion efficiency is between about 1% and about 25%. In some embodiments, the dispersion efficiency is about 15%.

In some embodiments, the dispersant compound comprises Compound 4. In some embodiments, Compound 4 comprises perfluoropropionic acid (PFPrA). In some embodiments, Compound 4 comprises the formula C₃HF₅O₂. In some embodiments, the dispersant compound comprises Formula II ((CF₂)_{X}CF₃COOH), wherein x is 1 (CF₂CF₃COOH), and the compound is Compound 4 comprising perfluoropropionic acid. In some embodiments, perfluoropropionic acid is 2,2,3,3,3-pentafluoropropanoic acid. In some embodiments, the molecular weight of Compound 4 is about 164 g/mol, or about the molecular weight of PFPrA. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 4. In some embodiments, the fluorinated solvent is perfluorohexane and the perfluorinated dispersant compound comprises Compound 4. In some embodiments, the fluorinated solvent is perfluorooctane and the perfluorinated dispersant compound comprises Compound 4. In some embodiments, the fluorinated solvent is perfluoropentane and the perfluorinated dispersant compound comprises Compound 4. In some embodiments, the fluorinated solvent is perfluoroheptane and the perfluorinated dispersant compound comprises Compound 4. In some embodiments, the fluorinated solvent is perfluoromethylcyclohexane and the perfluorinated dispersant compound comprises Compound 4. In some embodiments, the fluorinated solvent is perfluoro-1-3-dimethylcyclohexane and the perfluorinated dispersant compound comprises Compound 4. In some embodiments, the fluorinated solvent is perfluorodecalin and the perfluorinated dispersant compound comprises Compound 4.

In some embodiments, the protein of interest is dispersed in perfluorohexane in the presence of Compound 4. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 4, wherein the molar ratio of Compound 4 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 4, wherein the molar ratio of Compound 4 is about 1000:1 with the protein of interest. In some embodiments, the protein of interest phase separates when dispersed in perfluorohexane in the presence of Compound 4, but does not phase separate when dispersed in perfluorohexane in the absence of Compound 4. In some embodiments, the protein of interest phase separates when dispersed in perfluorohexane in the presence of Compound 4, wherein the dispersion efficiency is about 1%, 2%, 3%, 4%, 5%, 10%, 15%, 20%, or 25%. In some embodiments, the dispersion efficiency is between about 1% and about 25%. In some embodiments, the dispersion efficiency is about 15%.

In some embodiments, the dispersant compound comprises Compound 5. In some embodiments, Compound 5 comprises perfluoropentanoic acid (PFPeA). In some embodiments, Compound 5 comprises the formula C₅HF₉O₂. In some embodiments, the dispersant compound comprises Formula II ((CF₂)_{X}CF₃COOH), wherein x is 3 ((CF₂)₃CF₃COOH), and the compound is Compound 5 comprising perfluoropentanoic acid. In some embodiments, perfluoropentanoic acid is 2,2,3,3,4,4,5,5,5-nonafluoropentanoic acid. In some embodiments, the molecular weight of Compound 5 is about 264 g/mol, or about the molecular weight of PFPeA. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 5. In some embodiments, the fluorinated solvent is perfluorohexane and the perfluorinated dispersant compound comprises Compound 5. In some embodiments, the fluorinated solvent is perfluorooctane and the perfluorinated dispersant compound comprises Compound 5. In some embodiments, the fluorinated solvent is perfluoropentane and the perfluorinated dispersant compound comprises Compound 5. In some embodiments, the fluorinated solvent is perfluoroheptane and the perfluorinated dispersant compound comprises Compound 5. In some embodiments, the fluorinated solvent is perfluoromethylcyclohexane and the perfluorinated dispersant compound comprises Compound 5. In some embodiments, the fluorinated solvent is perfluoro-1-3-dimethylcyclohexane and the perfluorinated dispersant compound comprises Compound 5. In some embodiments, the fluorinated solvent is perfluorodecalin and the perfluorinated dispersant compound comprises Compound 5.

In some embodiments, the protein of interest is dispersed in perfluorohexane in the presence of Compound 5. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 5, wherein the molar ratio of Compound 5 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 5, wherein the molar ratio of Compound 5 is about 1000:1 with the protein of interest. In some embodiments, the protein of interest phase separates when dispersed in perfluorohexane in the presence of Compound 5, but does not phase separate when dispersed in perfluorohexane in the absence of Compound 5. In some embodiments, the protein of interest phase separates when dispersed in perfluorohexane in the presence of Compound 5, wherein the dispersion efficiency is about 1%, 2%, 3%, 4%, 5%, 10%, 15%, 20%, 25%, 30%, 35%, or 40%. In some embodiments, the dispersion efficiency is between about 1% and about 40%. In some embodiments, the dispersion efficiency is about 35%.

In some embodiments, the dispersant compound comprises Compound 6. In some embodiments, Compound 6 comprises perfluorooctanoic acid (PFOA). In some embodiments, Compound 6 comprises the formula C₈HF₁₅O₂. In some embodiments, the dispersant compound comprises Formula II ((CF₂)_{X}CF₃COOH), wherein x is 6 ((CF₂)₆CF₃COOH), and the compound is Compound 6 comprising perfluorooctanoic acid. In some embodiments, perfluorooctanoic acid is 2,2,3,3,4,4,5,5,6,6,7,7,8,8,8-pentadecafluorooctanoic acid. In some embodiments, the molecular weight of Compound 6 is about 414 g/mol, or about the molecular weight of PFOA. In some embodiments, Compound 6 comprises Formula II ((CF₂)_{X}CF₃COOH), wherein x is 6 and the compound is Compound 6 comprising perfluorooctanoic acid (PFOA). In some embodiments, Compound 6 is Compound 6. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 6. In some embodiments, the fluorinated solvent is perfluorohexane and the perfluorinated dispersant compound comprises Compound 6. In some embodiments, the fluorinated solvent is perfluorooctane and the perfluorinated dispersant compound comprises Compound 6. In some embodiments, the fluorinated solvent is perfluoropentane and the perfluorinated dispersant compound comprises Compound 6. In some embodiments, the fluorinated solvent is perfluoroheptane and the perfluorinated dispersant compound comprises Compound 6. In some embodiments, the fluorinated solvent is perfluoromethylcyclohexane and the perfluorinated dispersant compound comprises Compound 6. In some embodiments, the fluorinated solvent is perfluoro-1-3-dimethylcyclohexane and the perfluorinated dispersant compound comprises Compound 6. In some embodiments, the fluorinated solvent is perfluorodecalin and the perfluorinated dispersant compound comprises Compound 6.

In some embodiments, the protein of interest is dispersed in perfluorohexane in the presence of Compound 6. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 6, wherein the molar ratio of Compound 6 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 6, wherein the molar ratio of Compound 6 is about 1000:1 with the protein of interest. In some embodiments, the protein of interest phase separates when dispersed in perfluorohexane in the presence of Compound 6, but does not phase separate when dispersed in perfluorohexane in the absence of Compound 6. In some embodiments, the protein of interest phase separates when dispersed in perfluorohexane in the presence of Compound 6, wherein the dispersion efficiency is about 1%, 2%, 3%, 4%, 5%, 10%, 15%, 20%, 25%, 30%, 35%, or 40%. In some embodiments, the dispersion efficiency is between about 1% and about 40%. In some embodiments, the dispersion efficiency is about 35%.

In some embodiments, the dispersion efficiency of the protein of interest dissolved in PFH in the presence of PFOA is at least about 5%, 10%, 20%, 30%, 40%, 50%, 60%, 70%, 80%, or 90% when the protein of interest has a molecular weight of about 5 kDa to about 600 kDa. In some embodiments, the dispersion efficiency of the protein of interest is at least about 1%, 5%, 10%, 20%, 30%, 40%, 50%, 60%, 70%, 80%, or 90% when the protein of interest has a molecular weight of about 5 kDa to about 100 kDa. In some embodiments, the dispersion efficiency of the protein of interest dissolved in PFH in the presence of PFOA is at least about 35% when the protein of interest has a molecular weight of about 5 kDa to about 600 kDa.

In some embodiments, the dispersion efficiency of the protein of interest dissolved in PFH in the presence of PFOA is at least about 5%, 10%, 20%, 30%, 40%, 50%, 60%, 70%, 80%, or 90% when the solvent accessible surface area (SASA) is between about 1×10⁴ Å and about 15×10⁴ Å. In some embodiments, the dispersion efficiency of the protein of interest dissolved in PFH in the presence of PFOA is at least about 5%, 10%, 20%, 30%, 40%, 50%, 60%, 70%, 80%, or 90% when the hydrodynamic radii of the protein of interest is between about 2 and to 8 nm. In some embodiments, the dispersion efficiency of the protein of interest dissolved in PFH in the presence of PFOA is at least about 5%, 10%, 20%, 30%, 40%, 50%, 60%, 70%, 80%, or 90% when the isoelectric point (pI) of the protein of interest is between about 4 to about 8. In some embodiments, the dispersion efficiency of the protein of interest dissolved in PFH in the presence of PFOA is at least about 5%, 10%, 20%, 30%, 40%, 50%, 60%, 70%, 80%, or 90% when the α helicity content of the protein of interest is between 0% and about 35%. In some embodiments, the dispersion efficiency of the protein of interest dissolved in PFH in the presence of PFOA is at least about 5%, 10%, 20%, 30%, 40%, 50%, 60%, 70%, 80%, or 90% when the 0 sheet content of the protein of interest is between 0% and about 60%.

In some embodiments, the dispersion efficiency of the protein of interest dissolved in PFH in the presence of PFOA is between about 5% to about 100% when the protein of interest contains between about 0% to about 30% polar residues. In some embodiments, the dispersion efficiency of the protein of interest dissolved in PFH in the presence of PFOA is between about 35% to about 100% when the protein of interest contains between about 0% to about 30% polar residues. In some embodiments, the dispersion efficiency of the protein of interest dissolved in PFH in the presence of PFOA is between about 5% to about 100% when the protein of interest contains between about 0% to about 45% nonpolar residues. In some embodiments, the dispersion efficiency of the protein of interest dissolved in PFH in the presence of PFOA is between about 35% to about 100% when the protein of interest contains between about 0% to about 45% nonpolar residues.

In some embodiments, the protein of interest dissolved in PFH in the presence of PFOA, wherein the dispersant compound PFOA interacts with the protein of interest via hydrogen bonding with solvent shell H₂O molecules interacting with the protein of interest. In some embodiments, the protein of interest is dissolved in PFH in the presence of PFOA, wherein the dispersant compound PFOA interacts with the protein of interest via hydrogen bonding with nitrogen donors present in the protein backbone or the amino acid side chains of the protein of interest. In some embodiments, the protein of interest is dissolved in PFH in the presence of PFOA, wherein the dispersant compound PFOA interacts with the protein of interest via hydrogen bonding with oxygen donors present in the protein backbone or the amino acid side chains of the protein of interest. In some embodiments, the protein of interest is dissolved in PFH in the presence of PFOA, wherein the dispersant compound PFOA interacts with the protein of interest via hydrogen bonding with sulfur donors present in the amino acid side chains of the protein of interest. In some embodiments, the protein of interest dissolved in PFH in the presence of PFOA, wherein the carboxylic acid group of the dispersant compound PFOA interacts with the protein of interest via hydrogen bonding to the H₂O molecules interacting with the protein of interest. In some embodiments, the protein of interest dissolved in PFH in the presence of PFOA, wherein the carboxylic acid group of the dispersant compound PFOA interacts with the protein of interest via hydrogen bonding to the nitrogen, oxygen, or sulfur donors in the protein backbone or the amino acid side chains of the protein of interest. In some embodiments, the protein of interest dissolved in PFH in the presence of PFOA, wherein the one or more fluorine group of the dispersant compound PFOA interacts with the protein of interest via hydrogen bonding to the H₂O molecules interacting with the protein of interest. In some embodiments, the protein of interest dissolved in PFH in the presence of PFOA, wherein the one or more fluorine group of the dispersant compound PFOA interacts with the protein of interest via hydrogen bonding to the nitrogen, oxygen, or sulfur donors in the protein backbone or the amino acid side chains of the protein of interest.

In some embodiments, the protein of interest is dissolved in PFH in the presence of PFOA, wherein the dispersant compound PFOA makes hydrophobic contacts with protein solvent accessible amino acids. In some embodiments, the protein of interest is dissolved in PFH in the presence of PFOA, wherein the Gibbs free energy between the PFOA and the protein of interest is between about −20 to about −50 kcal/mol. In some embodiments, the protein of interest is dissolved in PFH in the presence of PFOA, wherein the Gibbs free energy between the PFOA and the docked rotamers from the protein of interest is between about −20 to about −50 kcal/mol.

In some embodiments, the structure of the protein of interest changes when dissolved in PFH in the presence of PFOA. In some embodiments, the protein of interest comprises a non-native secondary structure. In some embodiments, the change in molar ellipticity of the protein of interest is between about 2 deg cm⁻² dmol⁻¹ to about −6 deg cm⁻² dmol⁻¹. In some embodiments, the change in β-sheet content is between about −20% to about 20%. In some embodiments, the change in parallel β-sheet content is between about −20% to about 20%. In some embodiments, the change in antiparallel β-sheet content is between about −20% to about 20%. In some embodiments, the change in α-helix content is between about −20% to about 20%. In some embodiments, the change in turns content is between about −20% to about 20%. In some embodiments, the protein of interest is dissolved in PFH in the presence of PFOA, wherein the binding of the protein to the PFOA is 1:1. In some embodiments, the protein of interest is dissolved in PFH in the presence of PFOA, wherein the binding of the protein to the PFOA is not 1:1.

In some embodiments, the protein of interest is dissolved in PFH in the presence of PFOA, wherein the thermal stability of the protein is increased compared to the thermal stability of the protein of interest dissolved in an aqueous solvent. In some embodiments, the protein of interest is dissolved in PFH in the presence of PFOA, wherein the melting temperature of the protein is increased by about 5 °C, 10 °C, 20 °C, 30 °C, or 40 °C compared to the melting temperature of the protein of interest in an aqueous solvent. In some embodiments, the protein of interest is active in an aqueous solvent at 25 °C, but is not active in an aqueous solvent at 75 °C, 80 °C, 85 °C, or 90 °C. In some embodiments, the protein of interest is active in an aqueous solvent at 25 °C, but the activity is reduced when the protein of interest is dissolved in an aqueous solvent at 75 °C, 80 °C, 85 °C, or 90 °C. In some embodiments, the activity is reduced by about 80% or by about 90%. In some embodiments, the protein of interest is dissolved in PFH in the presence of PFOA, wherein the protein of interest is active at 25 °C and is also active at 75 °C, 80 °C, 85 °C, or 90 °C. In some embodiments, the protein of interest is dissolved in PFH in the presence of PFOA, wherein the activity is reduced by about 1% to about 25% when the temperature is about 75 °C, 80 °C, 85 °C, or 90 °C, compared to when the temperature is about 25 °C. In some embodiments, the protein of interest is dissolved in PFH in the presence of PFOA, wherein the activity is increased by about 1% to about 25% when the temperature is about 75 °C, 80 °C, 85 °C, or 90 °C, compared to when the temperature is about 25 °C.

In some embodiments, the protein of interest is dissolved in PFH in the presence of PFOA, wherein the protein comprises a reduced risk of contamination compared to when the protein is dissolved in an aqueous solvent. In some embodiments, the protein of interest is dissolved in PFH in the presence of PFOA, wherein the protein comprises a reduced risk of bacterial or viral contamination compared to when the protein is dissolved in PBS. In some embodiments, the protein of interest is dissolved in PFH in the presence of PFOA, wherein the protein comprises a reduced risk of E. coli contamination compared to when the protein is dissolved in PBS.

In some embodiments, the dispersant compound comprises Compound 7. In some embodiments, Compound 7 comprises perfluoronanonic acid (PFNA). In some embodiments, Compound 7 comprises the formula C₉HF₁₇O₂. In some embodiments, the dispersant compound comprises Formula II ((CF₂)_{X}CF₃COOH), wherein x is 7 ((CF₂)₇CF₃COOH), and the compound is Compound 7 comprising perfluoronanonic acid. In some embodiments, perfluoronanonic acid is 2,2,3,3,4,4,5,5,6,6,7,7,8,8,9,9,9-heptadecafluorononanoic acid. In some embodiments, the molecular weight of Compound 7 is about 464 g/mol, or about the molecular weight of PFNA. In some embodiments, Compound 7 comprises Formula II ((CF₂)_{X}CF₃COOH), wherein x is 7 ((CF₂)₇CF₃COOH), and the compound is Compound 7 comprising perfluoronanonic acid. In some embodiments, Compound 7 is Compound 7. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 7. In some embodiments, the fluorinated solvent is perfluorohexane and the perfluorinated dispersant compound comprises Compound 7. In some embodiments, the fluorinated solvent is perfluorooctane and the perfluorinated dispersant compound comprises Compound 7. In some embodiments, the fluorinated solvent is perfluoropentane and the perfluorinated dispersant compound comprises Compound 7. In some embodiments, the fluorinated solvent is perfluoroheptane and the perfluorinated dispersant compound comprises Compound 7. In some embodiments, the fluorinated solvent is perfluoromethylcyclohexane and the perfluorinated dispersant compound comprises Compound 7. In some embodiments, the fluorinated solvent is perfluoro-1-3-dimethylcyclohexane and the perfluorinated dispersant compound comprises Compound 7. In some embodiments, the fluorinated solvent is perfluorodecalin and the perfluorinated dispersant compound comprises Compound 7.

In some embodiments, the protein of interest is dispersed in perfluorohexane in the presence of Compound 7. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa.

In some embodiments, the protein of interest is dispersed in perfluorohexane in the presence of Compound 7, wherein the molar ratio of Compound 7 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 7, wherein the molar ratio of Compound 7 is about 1000:1 with the protein of interest. In some embodiments, the protein of interest phase separates when dispersed in perfluorohexane in the presence of Compound 7, but does not phase separate when dispersed in perfluorohexane in the absence of Compound 7. In some embodiments, the protein of interest phase separates when dispersed in perfluorohexane in the presence of Compound 7, wherein the dispersion efficiency is about 30%, 35%, 40%, 45%, 50%, 55%, 60%, 65%, 70%, 75%, 80%, 85%, 90%, 95%, or 100%. In some embodiments, the dispersion efficiency is about 50%. In some embodiments, the dispersion efficiency is about 55%. In some embodiments, the dispersion efficiency is about 60%. In some embodiments, the dispersion efficiency is about 65%. In some embodiments, the dispersion efficiency is about 75%. In some embodiments, the dispersion efficiency is about 80%. In some embodiments, the dispersion efficiency is about 85%. In some embodiments, the protein of interest phase separates when dispersed in perfluorohexane in the presence of Compound 7, wherein the dispersion efficiency is at least about 30%, about 40%, about 50%, about 60%, about 70%, about 80%, or at least about 90%. In some embodiments, the dispersion efficiency is at least 90%, 91%, 92%, 93%, 94%, 95%, 96%, 97%, 98%, or 99%.

In some embodiments, the dispersion efficiency of the protein of interest dissolved in PFH in the presence of PFNA is at least about 30%, 40%, 50%, 60%, 70%, 80%, or 90% when the protein of interest has a molecular weight of about 5 kDa to about 600 kDa. In some embodiments, the dispersion efficiency of the protein of interest is at least about 30%, 40%, 50%, 60%, 70%, 80%, or 90% when the protein of interest has a molecular weight of about 5 kDa to about 100 kDa. In some embodiments, the dispersion efficiency of the protein of interest is at least about 50%, 60%, 70%, 80%, or 90% when the protein of interest has a molecular weight of about 5 kDa to about 50 kDa. In some embodiments, the dispersion efficiency of the protein of interest is at least about 90%, 95%, 96%, 97%, 98%, or 99% when the protein of interest has a molecular weight of about 5 kDa to about 40 kDa. In some embodiments, the dispersion efficiency of the protein of interest dissolved in PFH in the presence of PFNA is at least about 55%, 60%, 70%, 80%, or 90% when the protein of interest has a molecular weight of about 5 kDa to about 500 kDa. In some embodiments, the dispersion efficiency of the protein of interest dissolved in PFH in the presence of PFNA is between about 55% to about 100 when the protein of interest has a molecular weight of about 5 kDa to about 500 kDa.

In some embodiments, the dispersion efficiency of the protein of interest dissolved in PFH in the presence of PFNA is at least about 30%, 40%, 50%, 60%, 70%, 80%, or 90% when the solvent accessible surface area (SASA) is between about 1×10⁴ Å and about 15×10⁴ Å. In some embodiments, the dispersion efficiency of the protein of interest dissolved in PFH in the presence of PFNA is at least about 50%, 60%, 70%, 80%, or 90% when the SASA is between about 1×10⁴ Å and about 15×10⁴ Å. In some embodiments, the dispersion efficiency of the protein of interest dissolved in PFH in the presence of PFNA is at least about 90%, 95%, 96%, 97%, 98%, or 99% when the SASA is between about 1×10⁴ Å and about 15×10⁴ Å. In some embodiments, the dispersion efficiency of the protein of interest dissolved in PFH in the presence of PFNA is at least about 90%, 95%, 96%, 97%, 98%, or 99% when the SASA is between about 1×10⁴ Å and about 2.5×10⁴ Å. In some embodiments, the dispersion efficiency of the protein of interest dissolved in PFH in the presence of PFNA is at least about 55%, 60%, 70%, 80%, or 90% when the SASA is between about 1×10⁴ Å and about 15×10⁴ Å. In some embodiments, the dispersion efficiency of the protein of interest dissolved in PFH in the presence of PFNA is between about 55% to about 100 when the SASA is between about 1×10⁴ Å and about 15.

In some embodiments, the dispersion efficiency of the protein of interest dissolved in PFH in the presence of PFNA is at least about 30%, 40%, 50%, 60%, 70%, 80%, or 90% when the hydrodynamic radii of the protein of interest is between about 2 and to 8 nm. In some embodiments, the dispersion efficiency of the protein of interest dissolved in PFH in the presence of PFNA is at least about 50%, 60%, 70%, 80%, or 90% when the hydrodynamic radii of the protein of interest is between about 2 to about 4 nm. In some embodiments, the dispersion efficiency of the protein of interest dissolved in PFH in the presence of PFNA is at least about 70%, 80%, or 90% when the hydrodynamic radii of the protein of interest is between about 2 to about 3 nm. In some embodiments, the dispersion efficiency of the protein of interest dissolved in PFH in the presence of PFNA is at least about 50%, 60%, 70%, 80%, or 90% when the hydrodynamic radii of the protein of interest is between about 6 to about 8 nm. In some embodiments, the dispersion efficiency of the protein of interest dissolved in PFH in the presence of PFNA is at least about 70%, 80%, or 90% when the hydrodynamic radii of the protein of interest is between about 7 to about 8 nm. In some embodiments, the dispersion efficiency of the protein of interest dissolved in PFH in the presence of PFNA is between about 30% to about 100% when the hydrodynamic radii of the protein of interest is between about 2 and to 8 nm. In some embodiments, the dispersion efficiency of the protein of interest dissolved in PFH in the presence of PFNA is between about 50% to about 100% when the hydrodynamic radii of the protein of interest is between about 2 to about 4 nm. In some embodiments, the dispersion efficiency of the protein of interest dissolved in PFH in the presence of PFNA is between about 70% to about 100% when the hydrodynamic radii of the protein of interest is between about 2 to about 3 nm. In some embodiments, the dispersion efficiency of the protein of interest dissolved in PFH in the presence of PFNA is between about 50% to about 100% when the hydrodynamic radii of the protein of interest is between about 6 to about 8 nm. In some embodiments, the dispersion efficiency of the protein of interest dissolved in PFH in the presence of PFNA is between about 70% to about 100% when the hydrodynamic radii of the protein of interest is between about 7 to about 8 nm. In some embodiments, the dispersion efficiency of the protein of interest dissolved in PFH in the presence of PFNA is at least 90, 91, 92%, 93%, 94%, 95%, 96%, 97%, 98%, or 99% when the hydrodynamic radii of the protein of interest is between about 2.5 to about 2.9 nm. In some embodiments, the dispersion efficiency of the protein of interest dissolved in PFH in the presence of PFNA is between about 90% to about 100% when the hydrodynamic radii of the protein of interest is between about 2.5 to about 2.9 nm. In some embodiments, the dispersion efficiency of the protein of interest dissolved in PFH in the presence of PFNA is about 95% when the hydrodynamic radii of the protein of interest is between about 2.5 to about 2.9 nm. In some embodiments, the dispersion efficiency of the protein of interest dissolved in PFH in the presence of PFNA is about 100% when the hydrodynamic radii of the protein of interest is between about 2.5 to about 2.9 nm. In some embodiments, the dispersion efficiency of the protein of interest dissolved in PFH in the presence of PFNA is at least 90, 91, 92%, 93%, 94%, 95%, 96%, 97%, 98%, or 99% when the hydrodynamic radii of the protein of interest is between about 6.5 to about 7.5 nm. In some embodiments, the dispersion efficiency of the protein of interest dissolved in PFH in the presence of PFNA is between about 90% to about 100% when the hydrodynamic radii of the protein of interest is between about 6.5 to about 7.5 nm. In some embodiments, the dispersion efficiency of the protein of interest dissolved in PFH in the presence of PFNA is about 95% when the hydrodynamic radii of the protein of interest is between about 6.5 to about 7.5 nm. In some embodiments, the dispersion efficiency of the protein of interest dissolved in PFH in the presence of PFNA is about 100% when the hydrodynamic radii of the protein of interest is between about 6.5 to about 7.5 nm. In some embodiments, the dispersion efficiency of the protein of interest dissolved in PFH in the presence of PFNA is at least about 55%, 60%, 70%, 80%, or 90% when the hydrodynamic radii of the protein of interest is between about 2 to about 8 nm. In some embodiments, the dispersion efficiency of the protein of interest dissolved in PFH in the presence of PFNA is between about 55% to about 100 when the hydrodynamic radii of the protein of interest is between about 2 to about 8 nm.

In some embodiments, the dispersion efficiency of the protein of interest dissolved in PFH in the presence of PFNA is at least about 30%, 40%, 50%, 60%, 70%, 80%, or 90% when the isoelectric point (pI) of the protein of interest is between about 4 to about 8. In some embodiments, the dispersion efficiency of the protein of interest dissolved in PFH in the presence of PFNA is at least about 50%, 60%, 70%, 80%, or 90% when the pI of the protein of interest is between about 4.5 to about 7.5. In some embodiments, the dispersion efficiency of the protein of interest dissolved in PFH in the presence of PFNA is at least about 90% when the pI of the protein of interest is between about 4.5 to about 5.5. In some embodiments, the dispersion efficiency of the protein of interest dissolved in PFH in the presence of PFNA is at least about 60%, 70%, 80%, or 90% when the pI of the protein of interest is between about 6.5 to about 7.5. In some embodiments, the dispersion efficiency of the protein of interest dissolved in PFH in the presence of PFNA is between about 30% to about 100% when the pI of the protein of interest is between about 4 to about 8. In some embodiments, the dispersion efficiency of the protein of interest dissolved in PFH in the presence of PFNA is at least about between about 50% to about 100% when the pI of the protein of interest is between about 4.5 to about 7.5. In some embodiments, the dispersion efficiency of the protein of interest dissolved in PFH in the presence of PFNA is between about 90% to about 100% when the pI of the protein of interest is between about 4.5 to about 5.5. In some embodiments, the dispersion efficiency of the protein of interest dissolved in PFH in the presence of PFNA is between about 60% to about 100% when the pI of the protein of interest is between about 6.5 to about 7.5. In some embodiments, the dispersion efficiency of the protein of interest dissolved in PFH in the presence of PFNA is at least about 90%, 91%, 92%, 93%, 94%, 95%, 96%, 97%, 98%, 99%, or about 100% when the pI of the protein of interest is between about 4.5 to about 5.5. In some embodiments, the dispersion efficiency of the protein of interest dissolved in PFH in the presence of PFNA is at least about 90% when the pI of the protein of interest is between about 4.5 to about 5.5. In some embodiments, the dispersion efficiency of the protein of interest dissolved in PFH in the presence of PFNA is at least about 95% when the pI of the protein of interest is between about 4.5 to about 5.5. In some embodiments, the dispersion efficiency of the protein of interest dissolved in PFH in the presence of PFNA is at least about 100% when the pI of the protein of interest is between about 4.5 to about 5.5. In some embodiments, the dispersion efficiency of the protein of interest dissolved in PFH in the presence of PFNA is at least about 30%, 40%, 50%, 60%, 70%, 80%, or 90% when the pI of the protein of interest is between about 4 to about 8. In some embodiments, the dispersion efficiency of the protein of interest dissolved in PFH in the presence of PFNA is at least about 55%, 60%, 70%, 80%, or 90% when the pI of the protein of interest is between about 4 to about 8. In some embodiments, the dispersion efficiency of the protein of interest dissolved in PFH in the presence of PFNA is between about 55% to about 100% when the pI of the protein of interest is between about 4 to about 8.

In some embodiments, the dispersion efficiency of the protein of interest dissolved in PFH in the presence of PFNA is at least about 30%, 40%, 50%, 60%, 70%, 80%, or 90% when the α helicity content of the protein of interest is between 0% and about 35%. In some embodiments, the dispersion efficiency of the protein of interest dissolved in PFH in the presence of PFNA is at least about 55%, 60%, 70%, 80%, or 90% when the α helicity content of the protein of interest is between 0% and about 35%. In some embodiments, the dispersion efficiency of the protein of interest dissolved in PFH in the presence of PFNA is between about 55% to about 100% when the α helicity content of the protein of interest is between 0 and about 35%. In some embodiments, the dispersion efficiency of the protein of interest dissolved in PFH in the presence of PFNA is at least about 55%, 60%, 70%, 80%, or 90% when the α helicity content of the protein of interest is between 0% and about 10%. In some embodiments, the dispersion efficiency of the protein of interest dissolved in PFH in the presence of PFNA is between about 55% to about 100% when the α helicity content of the protein of interest is between 0% and about 10%.

In some embodiments, the dispersion efficiency of the protein of interest dissolved in PFH in the presence of PFNA is at least about 30%, 40%, 50%, 60%, 70%, 80%, or 90% when the β sheet content of the protein of interest is between 0% and about 60%. In some embodiments, the dispersion efficiency of the protein of interest dissolved in PFH in the presence of PFNA is at least about 55%, 60%, 70%, 80%, or 90% when the β sheet content of the protein of interest is between about 10% and about 45%. In some embodiments, the dispersion efficiency of the protein of interest dissolved in PFH in the presence of PFNA is between about 55% to about 100% when the β sheet content of the protein of interest is between about 10% and about 45%. In some embodiments, the dispersion efficiency of the protein of interest dissolved in PFH in the presence of PFNA is at least about 55%, 60%, 70%, 80%, or 90% when the β sheet content of the protein of interest is between about 10% and about 45%. In some embodiments, the dispersion efficiency of the protein of interest dissolved in PFH in the presence of PFNA is between about 55% to about 100% when the R sheet content of the protein of interest is between about 10% and about 45%. In some embodiments, the dispersion efficiency of the protein of interest dissolved in PFH in the presence of PFNA is at least about 80%, or 90% when the β sheet content of the protein of interest is between about 35% and about 45%. In some embodiments, the dispersion efficiency of the protein of interest dissolved in PFH in the presence of PFNA is between about 80% to about 100% when the β sheet content of the protein of interest is between about 35% and about 45%. In some embodiments, the dispersion efficiency of the protein of interest dissolved in PFH in the presence of PFNA is at least about 85%, 90%, or 95% when the β sheet content of the protein of interest is between about 35% and about 45%. In some embodiments, the dispersion efficiency of the protein of interest dissolved in PFH in the presence of PFNA is between about 85% to about 100% when the 0 sheet content of the protein of interest is between about 35% and about 45%. In some embodiments, the dispersion efficiency of the protein of interest dissolved in PFH in the presence of PFNA is about 90% when the β sheet content of the protein of interest is between about 35% and about 45%. In some embodiments, the dispersion efficiency of the protein of interest dissolved in PFH in the presence of PFNA is about 95% when the β sheet content of the protein of interest is between about 35% and about 45%. In some embodiments, the dispersion efficiency of the protein of interest dissolved in PFH in the presence of PFNA is about 100% when the β sheet content of the protein of interest is between about 35% and about 45%.

In some embodiments, the dispersion efficiency of the protein of interest dissolved in PFH in the presence of PFNA is between about 40% to about 100% when the protein of interest contains between about 0% to about 30% polar residues. In some embodiments, the dispersion efficiency of the protein of interest dissolved in PFH in the presence of PFNA is between about 40% to about 100% when the protein of interest contains between about 0% to about 15% polar residues. In some embodiments, the dispersion efficiency of the protein of interest dissolved in PFH in the presence of PFNA is between about 40% to about 100% when the protein of interest contains about 0.5%, 1%, 2%, 3%, 4%, 5%, 6%, 7%, 8%, 9%, 10%, 11%, 12%, 13%, 14%, or 15% polar residues.

In some embodiments, the dispersion efficiency of the protein of interest dissolved in PFH in the presence of PFNA is between about 40% to about 100% when the protein of interest contains between about 0% to about 15% polar residues, wherein the polar residues comprise lysine, arginine, histidine, aspartate, or glutamate, or any combination thereof. In some embodiments, the dispersion efficiency of the protein of interest dissolved in PFH in the presence of PFNA is between about 40% to about 100% when the protein of interest contains between about 0% to about 15% polar charged residues, wherein the polar charged residues comprise lysine, arginine, histidine, aspartate, or glutamate, or any combination thereof. In some embodiments, the dispersion efficiency of the protein of interest dissolved in PFH in the presence of PFNA is between about 40% to about 100% when the protein of interest contains between about 0% to about 15% polar residues, wherein the polar residues comprise serine, threonine, cysteine, asparagine, or glutamine, or any combination thereof. In some embodiments, the dispersion efficiency of the protein of interest dissolved in PFH in the presence of PFNA is between about 40% to about 100% when the protein of interest contains between about 0% to about 15% polar uncharged residues, wherein the polar uncharged residues comprise lysine, arginine, histidine, asparagine, or glutamine, or any combination thereof. In some embodiments, the dispersion efficiency of the protein of interest dissolved in PFH in the presence of PFNA is between about 40% to about 100% when the protein of interest contains between about 0% to about 15% polar residues, wherein the polar residues comprise lysine, arginine, histidine, aspartate, glutamate, lysine, arginine, histidine, asparagine, or glutamine, or any combination thereof.

In some embodiments, the dispersion efficiency of the protein of interest dissolved in PFH in the presence of PFNA is between about 40% to about 100% when the protein of interest contains between about 0% to about 15% lysine residues. In some embodiments, the dispersion efficiency of the protein of interest dissolved in PFH in the presence of PFNA is between about 40% to about 100% when the protein of interest contains between about 0% to about 15% arginine residues. In some embodiments, the dispersion efficiency of the protein of interest dissolved in PFH in the presence of PFNA is between about 40% to about 100% when the protein of interest contains between about 0% to about 15% histidine residues. In some embodiments, the dispersion efficiency of the protein of interest dissolved in PFH in the presence of PFNA is between about 40% to about 100% when the protein of interest contains between about 0% to about 15% aspartate residues. In some embodiments, the dispersion efficiency of the protein of interest dissolved in PFH in the presence of PFNA is between about 40% to about 100% when the protein of interest contains between about 0% to about 15% glutamate residues. In some embodiments, the dispersion efficiency of the protein of interest dissolved in PFH in the presence of PFNA is between about 40% to about 100% when the protein of interest contains between about 0% to about 15% serine residues. In some embodiments, the dispersion efficiency of the protein of interest dissolved in PFH in the presence of PFNA is between about 40% to about 100% when the protein of interest contains between about 0% to about 15% threonine residues. In some embodiments, the dispersion efficiency of the protein of interest dissolved in PFH in the presence of PFNA is between about 40% to about 100% when the protein of interest contains between about 0% to about 15% cystine residues. In some embodiments, the dispersion efficiency of the protein of interest dissolved in PFH in the presence of PFNA is between about 40% to about 100% when the protein of interest contains between about 0% to about 15% asparagine residues. In some embodiments, the dispersion efficiency of the protein of interest dissolved in PFH in the presence of PFNA is between about 40% to about 100% when the protein of interest contains between about 0% to about 15% glutamine residues.

In some embodiments, the dispersion efficiency of the protein of interest dissolved in PFH in the presence of PFNA is between about 40% to about 100% when the protein of interest contains between about 1% to about 12% lysine residues. In some embodiments, the dispersion efficiency of the protein of interest dissolved in PFH in the presence of PFNA is between about 40% to about 100% when the protein of interest contains between about 1% to about 8% arginine residues. In some embodiments, the dispersion efficiency of the protein of interest dissolved in PFH in the presence of PFNA is between about 40% to about 100% when the protein of interest contains between about 1% to about 8% histidine residues. In some embodiments, the dispersion efficiency of the protein of interest dissolved in PFH in the presence of PFNA is between about 40% to about 100% when the protein of interest contains between about 3% to about 8% aspartate residues. In some embodiments, the dispersion efficiency of the protein of interest dissolved in PFH in the presence of PFNA is between about 40% to about 100% when the protein of interest contains between about 3% to about 11% glutamate residues. In some embodiments, the dispersion efficiency of the protein of interest dissolved in PFH in the presence of PFNA is between about 40% to about 100% when the protein of interest contains between about 3% to about 15% serine residues. In some embodiments, the dispersion efficiency of the protein of interest dissolved in PFH in the presence of PFNA is between about 40% to about 100% when the protein of interest contains between about 4% to about 10% threonine residues. In some embodiments, the dispersion efficiency of the protein of interest dissolved in PFH in the presence of PFNA is between about 40% to about 100% when the protein of interest contains between about 0.5% to about 7% cysteine residues. In some embodiments, the dispersion efficiency of the protein of interest dissolved in PFH in the presence of PFNA is between about 40% to about 100% when the protein of interest contains between about 2% to about 7% asparagine residues. In some embodiments, the dispersion efficiency of the protein of interest dissolved in PFH in the presence of PFNA is between about 40% to about 100% when the protein of interest contains between about 1% to about 6% glutamine residues.

In some embodiments, the dispersion efficiency of the protein of interest dissolved in PFH in the presence of PFNA is between about 40% to about 100% when the protein of interest contains between about 0% to about 45% nonpolar residues. In some embodiments, the dispersion efficiency of the protein of interest dissolved in PFH in the presence of PFNA is between about 40% to about 100% when the protein of interest contains between about 0% to about 30% nonpolar residues. In some embodiments, the dispersion efficiency of the protein of interest dissolved in PFH in the presence of PFNA is between about 40% to about 100% when the protein of interest contains between about 0% to about 15% nonpolar residues. In some embodiments, the dispersion efficiency of the protein of interest dissolved in PFH in the presence of PFNA is between about 40% to about 100% when the protein of interest contains about 0.5%, 1%, 2%, 3%, 4%, 5%, 6%, 7%, 8%, 9%, 10%, 11%, 12%, 13%, 14%, or 15% nonpolar residues. In some embodiments, the dispersion efficiency of the protein of interest dissolved in PFH in the presence of PFNA is between about 40% to about 100% when the protein of interest contains between about 0% to about 15% nonpolar residues, wherein the nonpolar residues comprise glycine, alanine, valine, leucine, isoleucine, methionine, phenylalanine, tyrosine, tryptophan, or proline, or any combination thereof.

In some embodiments, the dispersion efficiency of the protein of interest dissolved in PFH in the presence of PFNA is between about 40% to about 100% when the protein of interest contains between about 0% to about 15% glycine residues. In some embodiments, the dispersion efficiency of the protein of interest dissolved in PFH in the presence of PFNA is between about 40% to about 100% when the protein of interest contains between about 0% to about 15% alanine residues. In some embodiments, the dispersion efficiency of the protein of interest dissolved in PFH in the presence of PFNA is between about 40% to about 100% when the protein of interest contains between about 0% to about 15% valine residues. In some embodiments, the dispersion efficiency of the protein of interest dissolved in PFH in the presence of PFNA is between about 40% to about 100% when the protein of interest contains between about 0% to about 15% leucine residues. In some embodiments, the dispersion efficiency of the protein of interest dissolved in PFH in the presence of PFNA is between about 40% to about 100% when the protein of interest contains between about 0% to about 15% isoleucine residues. In some embodiments, the dispersion efficiency of the protein of interest dissolved in PFH in the presence of PFNA is between about 40% to about 100% when the protein of interest contains between about 0% to about 15% methionine residues. In some embodiments, the dispersion efficiency of the protein of interest dissolved in PFH in the presence of PFNA is between about 40% to about 100% when the protein of interest contains between about 0% to about 15% phenylalanine residues. In some embodiments, the dispersion efficiency of the protein of interest dissolved in PFH in the presence of PFNA is between about 40% to about 100% when the protein of interest contains between about 0% to about 15% tyrosine residues. In some embodiments, the dispersion efficiency of the protein of interest dissolved in PFH in the presence of PFNA is between about 40% to about 100% when the protein of interest contains between about 0% to about 15% tryptophan residues. In some embodiments, the dispersion efficiency of the protein of interest dissolved in PFH in the presence of PFNA is between about 40% to about 100% when the protein of interest contains between about 0% to about 15% proline residues.

In some embodiments, the dispersion efficiency of the protein of interest dissolved in PFH in the presence of PFNA is between about 40% to about 100% when the protein of interest contains between about 2% to about 10% glycine residues. In some embodiments, the dispersion efficiency of the protein of interest dissolved in PFH in the presence of PFNA is between about 40% to about 100% when the protein of interest contains between about 2% to about 15% alanine residues. In some embodiments, the dispersion efficiency of the protein of interest dissolved in PFH in the presence of PFNA is between about 40% to about 100% when the protein of interest contains between about 4% to about 12% valine residues. In some embodiments, the dispersion efficiency of the protein of interest dissolved in PFH in the presence of PFNA is between about 40% to about 100% when the protein of interest contains between about 4% to about 14% leucine residues. In some embodiments, the dispersion efficiency of the protein of interest dissolved in PFH in the presence of PFNA is between about 40% to about 100% when the protein of interest contains between about 0% to about 6% isoleucine residues. In some embodiments, the dispersion efficiency of the protein of interest dissolved in PFH in the presence of PFNA is between about 40% to about 100% when the protein of interest contains between about 0.25% to about 3% methionine residues. In some embodiments, the dispersion efficiency of the protein of interest dissolved in PFH in the presence of PFNA is between about 40% to about 100% when the protein of interest contains between about 1.5% to about 6% phenylalanine residues. In some embodiments, the dispersion efficiency of the protein of interest dissolved in PFH in the presence of PFNA is between about 40% to about 100% when the protein of interest contains between about 1% to about 5% tyrosine residues. In some embodiments, the dispersion efficiency of the protein of interest dissolved in PFH in the presence of PFNA is between about 40% to about 100% when the protein of interest contains between about 0% to about 5% tryptophan residues. In some embodiments, the dispersion efficiency of the protein of interest dissolved in PFH in the presence of PFNA is between about 40% to about 100% when the protein of interest contains between about 3% to about 8% proline residues.

In some embodiments, the protein of interest dissolved in PFH in the presence of PFNA, wherein the dispersant compound PFNA interacts with the protein of interest via hydrogen bonding with solvent shell H₂O molecules interacting with the protein of interest. In some embodiments, the protein of interest is dissolved in PFH in the presence of PFNA, wherein the dispersant compound PFNA interacts with the protein of interest via hydrogen bonding with nitrogen donors present in the protein backbone of the protein of interest.

In some embodiments, the protein of interest is dissolved in PFH in the presence of PFNA, wherein the dispersant compound PFNA interacts with the protein of interest via hydrogen bonding with nitrogen donors present in the protein backbone of the protein of interest, wherein the amino acid comprises arginine, histidine, lysine, aspartate, glutamate, asparagine, glutamine, serine, threonine, cysteine, glycine, proline, alanine, valine, isoleucine, leucine, methionine, phenylalanine, tyrosine, or tryptophan. In some embodiments, the protein of interest is dissolved in PFH in the presence of PFNA, wherein the dispersant compound PFNA interacts with the protein of interest via hydrogen bonding with nitrogen donors present in the protein backbone of the protein of interest, wherein the amino acid comprises serine, threonine, cysteine, methionine, tyrosine, tryptophan, aspartate, glutamate, asparagine, glutamine, histidine, leucine, or arginine.

In some embodiments, the protein of interest is dissolved in PFH in the presence of PFNA, wherein the dispersant compound PFNA interacts with the protein of interest via hydrogen bonding with oxygen donors present in the protein backbone of the protein of interest, wherein the amino acid comprises arginine, histidine, lysine, aspaitate, glutamate, asparagine, glutamine, serine, threonine, cysteine, glycine, proline, alanine, valine, isoleucine, leucine, methionine, phenylalanine, tyrosine, or tryptophan. In some embodiments, the protein of interest is dissolved in PFH in the presence of PFNA, wherein the dispersant compound PFNA interacts with the protein of interest via hydrogen bonding with oxygen donors present in the protein backbone of the protein of interest, wherein the amino acid comprises serine, threonine, cysteine, methionine, tyrosine, tryptophan, aspartate, glutamate, asparagine, glutamine, histidine, leucine, or arginine.

In some embodiments, the protein of interest is dissolved in PFH in the presence of PFNA, wherein the dispersant compound PFNA interacts with the protein of interest via hydrogen bonding with nitrogen donors present in the amino acid side chains of the protein of interest wherein the amino acid comprises tryptophan, asparagine, glutamine, histidine, or arginine. In some embodiments, the protein of interest is dissolved in PFH in the presence of PFNA, wherein the dispersant compound PFNA interacts with the protein of interest via hydrogen bonding with nitrogen donors present in the protein backbone and amino acid side chains of the protein of interest, wherein the amino acid comprises tryptophan, asparagine, glutamine, histidine, or arginine.

In some embodiments, the protein of interest is dissolved in PFH in the presence of PFNA, wherein the dispersant compound PFNA interacts with the protein of interest via hydrogen bonding with oxygen donors present in the amino acid side chains of the protein of interest, wherein the amino acid comprises serine, threonine, tyrosine, aspartate, glutamate, asparagine, or glutamine. In some embodiments, the protein of interest is dissolved in PFH in the presence of PFNA, wherein the dispersant compound PFNA interacts with the protein of interest via hydrogen bonding with oxygen donors present in the protein backbone and amino acid side chains of the protein of interest, wherein the amino acid comprises serine, threonine, tyrosine, aspartate, glutamate, asparagine, or glutamine.

In some embodiments, the protein of interest is dissolved in PFH in the presence of PFNA, wherein the dispersant compound PFNA interacts with the protein of interest via hydrogen bonding with sulfur donors present in the amino acid side chains of the protein of interest, wherein the amino acid comprises cysteine or methionine.

In some embodiments, the protein of interest dissolved in PFH in the presence of PFNA, wherein the carboxylic acid group of the dispersant compound PFNA interacts with the protein of interest via hydrogen bonding to the H₂O molecules interacting with the protein of interest. In some embodiments, the protein of interest dissolved in PFH in the presence of PFNA, wherein the carboxylic acid group of the dispersant compound PFNA interacts with the protein of interest via hydrogen bonding to the nitrogen donors present in the protein backbone of the protein of interest. In some embodiments, the protein of interest dissolved in PFH in the presence of PFNA, wherein the carboxylic acid group of the dispersant compound PFNA interacts with the protein of interest via hydrogen bonding to the oxygen donors present in the amino acid side chains of the protein of interest. In some embodiments, the amino acid is serine, threonine, tyrosine, aspartate, or glutamate. In some embodiments, the protein of interest dissolved in PFH in the presence of PFNA, wherein the carboxylic acid group of the dispersant compound PFNA interacts with the protein of interest via hydrogen bonding to the nitrogen donors present in the amino acid side chains of the protein of interest. In some embodiments, the amino acid is histidine. In some embodiments, the protein of interest dissolved in PFH in the presence of PFNA, wherein the carboxylic acid group of the dispersant compound PFNA interacts with the protein of interest via hydrogen bonding to the sulfur donors present in the amino acid side chains of the protein of interest.

In some embodiments, the protein of interest dissolved in PFH in the presence of PFNA, wherein one or more fluorine group of the dispersant compound PFNA interacts with the protein of interest via hydrogen bonding to the H₂O molecules interacting with the protein of interest. In some embodiments, the protein of interest dissolved in PFH in the presence of PFNA, wherein one or more fluorine group of the dispersant compound PFNA interacts with the protein of interest via hydrogen bonding to the nitrogen donors present in the protein backbone of the protein of interest. In some embodiments, the protein of interest dissolved in PFH in the presence of PFNA, wherein one or more fluorine group of the dispersant compound PFNA interacts with the protein of interest via hydrogen bonding to the oxygen donors present in the amino acid side chains of the protein of interest. In some embodiments, the amino acid is serine, threonine, tyrosine, asparagine, or glutamine. In some embodiments, the amino acid is serine, threonine, or tyrosine. In some embodiments, the protein of interest dissolved in PFH in the presence of PFNA, wherein one or more fluorine group of the dispersant compound PFNA interacts with the protein of interest via hydrogen bonding to the nitrogen donors present in the amino acid side chains of the protein of interest. In some embodiments, the amino acid is histidine or arginine. In some embodiments, the protein of interest dissolved in PFH in the presence of PFNA, wherein one or more fluorine group of the dispersant compound PFNA interacts with the protein of interest via hydrogen bonding to the sulfur donors present in the amino acid side chains of the protein of interest.

In some embodiments, the protein of interest is dissolved in PFH in the presence of PFNA, wherein the dispersant compound PFNA makes hydrophobic contacts with protein solvent accessible amino acids. In some embodiments, the protein of interest is dissolved in PFH in the presence of PFNA, wherein the dispersant compound PFNA makes hydrophobic contacts with protein solvent accessible amino acids, wherein the solvent accessible amino acid is G, A, S, T, C, V, L, I, M, P, F, Y, W, D, E, N, Q, H, L, or R. In some embodiments, the solvent accessible amino acid is G, A, S, T, V, L, I, P, F, Y, W, D, E, N, Q, H, L, or R. In some embodiments, the solvent accessible amino acid is S, T, Y, D, H, L, or R.

In some embodiments, the protein of interest is dissolved in PFH in the presence of PFNA, wherein the Gibbs free energy between the PFNA and the protein of interest is between about −20 to about −50 kcal/mol. In some embodiments, the protein of interest is dissolved in PFH in the presence of PFNA, wherein the Gibbs free energy between the PFNA and the docked rotamers from the protein of interest is between about −20 to about −50 kcal/mol.

In some embodiments, the structure of the protein of interest changes when dissolved in PFH in the presence of PFNA. In some embodiments, the protein of interest comprises a non-native secondary structure. In some embodiments, the change in molar ellipticity of the protein of interest is between about 2 deg cm⁻² dmol⁻¹ to about −6 deg cm⁻² dmol⁻¹. In some embodiments, the change in molar ellipticity of the protein of interest is between about 1 deg cm⁻² dmol⁻¹ to about −1 deg cm⁻² dmol⁻¹. In some embodiments, the molar ellipticity of the protein of interest decreases by about −1 deg cm⁻² dmol⁻¹ to about −6 deg cm⁻² dmol⁻¹. In some embodiments, the molar ellipticity of the protein of interest increases by about 1 deg cm⁻² dmol⁻¹ to about 2 deg cm⁻² dmol⁻¹. In some embodiments, the change in β-sheet content is between about −20% to about 20%. In some embodiments, the change in parallel β-sheet content is between about −20% to about 20%. In some embodiments, the change in antiparallel β-sheet content is between about −20% to about 20%. In some embodiments, the change in α-helix content is between about −20% to about 20%. In some embodiments, the change in turns content is between about −20% to about 20%. In some embodiments, the change in β-sheet content is between about −8% to about 8%. In some embodiments, the change in parallel β-sheet content is between about −8% to about 8%. In some embodiments, the change in antiparallel β-sheet content is between about −8% to about 3%. In some embodiments, the change in α-helix content is between about −1% to about 5%. In some embodiments, the change in turn content is between about −6% to about 1%.

In some embodiments, the protein of interest is dissolved in PFH in the presence of PFNA, wherein the binding of the protein to the PFNA is 1:1. In some embodiments, the protein of interest is dissolved in PFH in the presence of PFNA, wherein the binding of the protein to the PFNA is not 1:1.

In some embodiments, the protein of interest is dissolved in PFH in the presence of PFNA, wherein the thermal stability of the protein is increased compared to the thermal stability of the protein of interest dissolved in an aqueous solvent. In some embodiments, the protein of interest is dissolved in PFH in the presence of PFNA, wherein the melting temperature of the protein is increased by about 10 °C, 20 °C, 30 °C, or 40 °C compared to the melting temperature of the protein of interest in an aqueous solvent. In some embodiments, the protein of interest is active in an aqueous solvent at 25 °C, but is not active in an aqueous solvent at 75 °C, 80 °C, 85 °C, or 90 °C. In some embodiments, the protein of interest is active in an aqueous solvent at 25 °C, but the activity is reduced when the protein of interest is dissolved in an aqueous solvent at 75 °C, 80 °C, 85 °C, or 90 GC. In some embodiments, the activity is reduced by about 80% or by about 90%. In some embodiments, the protein of interest is dissolved in PFH in the presence of PFNA, wherein the protein of interest is active at 25 °C and is also active at 75 °C, 80 °C, 85 °C, or 90 °C. In some embodiments, the protein of interest is dissolved in PFH in the presence of PFNA, wherein the activity is reduced by about 1% to about 25% when the temperature is about 75 °C, 80 °C, 85 °C, or 90 °C, compared to when the temperature is about 25 GC. In some embodiments, the protein of interest is dissolved in PFH in the presence of PFNA, wherein the activity is reduced by about 1% to about 10% when the temperature is about 75 °C, 80 °C, 85 °C, or 90 °C, compared to when the temperature is about 25 GC. In some embodiments, the protein of interest is dissolved in PFH in the presence of PFNA, wherein the activity is increased by about 1% to about 25% when the temperature is about 75 °C, 80 °C, 85 °C, or 90 °C, compared to when the temperature is about 25 GC. In some embodiments, the protein of interest is dissolved in PFH in the presence of PFNA, wherein the activity is increased by about 1% to about 10% when the temperature is about 75 °C, 80 °C, 85 °C, or 90 °C, compared to when the temperature is about 25 GC.

In some embodiments, the protein of interest is dissolved in PFH in the presence of PFNA, wherein the protein comprises a reduced risk of contamination compared to when the protein is dissolved in an aqueous solvent. In some embodiments, the protein of interest is dissolved in PFH in the presence of PFNA, wherein the protein comprises a reduced risk of bacterial or viral contamination compared to when the protein is dissolved in PBS. In some embodiments, the protein of interest is dissolved in PFH in the presence of PFNA, wherein the protein comprises a reduced risk of E. coli contamination compared to when the protein is dissolved in PBS.

In some embodiments, the dispersant compound comprises Compound 8. In some embodiments, Compound 8 comprises perfluorodecanoic acid (PFDA). In some embodiments, Compound 8 comprises the formula C₁₀HF₁₉O₂. In some embodiments, the dispersant compound comprises Formula II ((CF₂)_{X}CF₃COOH), wherein x is 8 ((CF₂)₈CF₃COOH), and the compound is Compound 8 comprising perfluorodecanoic acid. In some embodiments, perfluorodecanoic acid is 2,2,3,3,4,4,5,5,6,6,7,7,8,8,9,9,10,10,10-nonadecafluorodecanoic acid. In some embodiments, the molecular weight of Compound 8 is about 514 g/mol, or about the molecular weight of PFDA. In some embodiments, Compound 8 comprises Formula II ((CF₂)_{X}CF₃COOH), wherein x is 8 ((CF₂)₈CF₃COOH), and the compound is Compound 8 comprising perfluorodecanoic acid. In some embodiments, Compound 8 is Compound 8. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 8. In some embodiments, the fluorinated solvent is perfluorohexane and the perfluorinated dispersant compound comprises Compound 8. In some embodiments, the fluorinated solvent is perfluorooctane and the perfluorinated dispersant compound comprises Compound 8. In some embodiments, the fluorinated solvent is perfluoropentane and the perfluorinated dispersant compound comprises Compound 8. In some embodiments, the fluorinated solvent is perfluoroheptane and the perfluorinated dispersant compound comprises Compound 8. In some embodiments, the fluorinated solvent is perfluoromethylcyclohexane and the perfluorinated dispersant compound comprises Compound 8. In some embodiments, the fluorinated solvent is perfluoro-1-3-dimethylcyclohexane and the perfluorinated dispersant compound comprises Compound 8. In some embodiments, the fluorinated solvent is perfluorodecalin and the perfluorinated dispersant compound comprises Compound 8.

In some embodiments, the protein of interest is dispersed in perfluorohexane in the presence of Compound 8. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 8, wherein the molar ratio of Compound 8 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 8, wherein the molar ratio of Compound 8 is about 1000:1 with the protein of interest. In some embodiments, the protein of interest phase separates when dispersed in perfluorohexane in the presence of Compound 8, but does not phase separate when dispersed in perfluorohexane in the absence of Compound 8. In some embodiments, the protein of interest phase separates when dispersed in perfluorohexane in the presence of Compound 8, wherein the dispersion efficiency is about 10%, 15%, 20%, 25%, 30%, 35%, 40%, 45%, 50%, 55%, 60%, 65%, 70%, 75%, or about 80%. In some embodiments, the dispersion efficiency is between about 10% and about 80%. In some embodiments, the dispersion efficiency is about 80%. In some embodiments, the dispersion efficiency is about 50%. In some embodiments, the dispersion efficiency is about 40%.

In some embodiments, the dispersant compound comprises Compound 9. In some embodiments, Compound 9 comprises perfluoroundecanoic acid (PFUnDA). In some embodiments, Compound 9 comprises the formula C₁₁HF₂₁O₂. In some embodiments, the dispersant compound comprises Formula II ((CF₂)_{X}CF₃COOH), wherein x is 9 ((CF₂)₉CF₃COOH), and the compound is Compound 9 comprising perfluoroundecanoic acid. In some embodiments, perfluoroundecanoic acid is 2,2,3,3,4,4,5,5,6,6,7,7,8,8,9,9,10,10,11,11,11-henicosafluoroundecanoic acid. In some embodiments, the molecular weight of Compound 9 is about 564 g/mol, or about the molecular weight of PFUnDA. In some embodiments, Compound 9 comprises Formula II ((CF₂)_{X}CF₃COOH), wherein x is 9 ((CF₂)₉CF₃COOH), and the compound is Compound 9 comprising perfluoroundecanoic acid. In some embodiments, Compound 9 is Compound 9. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 9. In some embodiments, the fluorinated solvent is perfluorohexane and the perfluorinated dispersant compound comprises Compound 9. In some embodiments, the fluorinated solvent is perfluorooctane and the perfluorinated dispersant compound comprises Compound 9. In some embodiments, the fluorinated solvent is perfluoropentane and the perfluorinated dispersant compound comprises Compound 9. In some embodiments, the fluorinated solvent is perfluoroheptane and the perfluorinated dispersant compound comprises Compound 9. In some embodiments, the fluorinated solvent is perfluoromethylcyclohexane and the perfluorinated dispersant compound comprises Compound 9. In some embodiments, the fluorinated solvent is perfluoro-1-3-dimethylcyclohexane and the perfluorinated dispersant compound comprises Compound 9. In some embodiments, the fluorinated solvent is perfluorodecalin and the perfluorinated dispersant compound comprises Compound 9.

In some embodiments, the protein of interest is dispersed in perfluorohexane in the presence of Compound 9. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 9, wherein the molar ratio of Compound 9 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 9, wherein the molar ratio of Compound 9 is about 1000:1 with the protein of interest. In some embodiments, the protein of interest phase separates when dispersed in perfluorohexane in the presence of Compound 9, but does not phase separate when dispersed in perfluorohexane in the absence of Compound 9. In some embodiments, the protein of interest phase separates when dispersed in perfluorohexane in the presence of Compound 9, wherein the dispersion efficiency is about 5%, 10%, 15%, 20%, 25%, 30%, 35%, 40%, 45%, 50%, 55%, 60%, 65%, 70%, 75%, or about 80%. In some embodiments, the dispersion efficiency is between about 5% and about 80%. In some embodiments, the dispersion efficiency is about 80%. In some embodiments, the dispersion efficiency is about 50%. In some embodiments, the dispersion efficiency is about 25%.

In some embodiments, the dispersant compound comprises Compound 10. In some embodiments, Compound 10 comprises perfluorododecanoic acid. In some embodiments, Compound 10 comprises the formula C₁₂HF₂₃O₂. In some embodiments, the dispersant compound comprises Formula II ((CF₂)_{X}(CF₃)₂COOH), wherein x is 10 ((CF₂)₁₀CF₃COOH), and the compound is Compound 10 comprising perfluorododecanoic acid. In some embodiments, perfluorododecanoic acid is 2,2,3,3,4,4,5,5,6,6,7,7,8,8,9,9,10,10,11,11,12,12,12-tricosafluorododecanoic acid. In some embodiments, the molecular weight of Compound 10 is about 614 g/mol, or about the molecular weight of perfluorododecanoic acid. In some embodiments, Compound 10 comprises Formula II ((CF₂)_{X}(CF₃)₂COOH), wherein x is 10 ((CF₂)₁₀CF₃COOH), and the compound is Compound 10 comprising perfluorododecanoic acid. In some embodiments, Compound 10 is Compound 10. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 10. In some embodiments, the fluorinated solvent is perfluorohexane and the perfluorinated dispersant compound comprises Compound 10. In some embodiments, the fluorinated solvent is perfluorooctane and the perfluorinated dispersant compound comprises Compound 10. In some embodiments, the fluorinated solvent is perfluoropentane and the perfluorinated dispersant compound comprises Compound 10. In some embodiments, the fluorinated solvent is perfluoroheptane and the perfluorinated dispersant compound comprises Compound 10. In some embodiments, the fluorinated solvent is perfluoromethylcyclohexane and the perfluorinated dispersant compound comprises Compound 10. In some embodiments, the fluorinated solvent is perfluoro-1-3-dimethylcyclohexane and the perfluorinated dispersant compound comprises Compound 10. In some embodiments, the fluorinated solvent is perfluorodecalin and the perfluorinated dispersant compound comprises Compound 10.

In some embodiments, the protein of interest is dispersed in perfluorohexane in the presence of Compound 10. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 10, wherein the molar ratio of Compound 10 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 10, wherein the molar ratio of Compound 10 is about 1000:1 with the protein of interest. In some embodiments, the protein of interest phase separates when dispersed in perfluorohexane in the presence of Compound 10, but does not phase separate when dispersed in perfluorohexane in the absence of Compound 10. In some embodiments, the protein of interest phase separates when dispersed in perfluorohexane in the presence of Compound 10, wherein the dispersion efficiency is about 5%, 10%, 15%, 20%, 25%, 30%, 35%, 40%, 45%, 50%, 55%, 60%, 65%, 70%, 75%, or about 80%. In some embodiments, the dispersion efficiency is between about 5% and about 80%. In some embodiments, the dispersion efficiency is about 80%. In some embodiments, the dispersion efficiency is about 50%. In some embodiments, the dispersion efficiency is about 25%. In some embodiments, the dispersion efficiency is about 15%.

In some embodiments, the dispersant compound comprises Compound 11. In some embodiments, Compound 11 comprises perfluorotetradecanoic acid (PFTeDA). In some embodiments, Compound 11 comprises the formula C₁₃HF₂₇O₂. In some embodiments, the dispersant compound comprises Formula II ((CF₂)_{X}(CF₃)₂COOH), wherein x is 12 ((CF₂)₁₂CF₃COOH), and the compound is Compound 11 comprising perfluorotetradecanoic acid. In some embodiments, perfluorotetradecanoic acid is 2,2,3,3,4,4,5,5,6,6,7,7,8,8,9,9,10,10,11,11,12,12,13,13,14,14,14-heptacosafluorotetradecanoic acid. In some embodiments, the molecular weight of Compound 11 is about 714 g/mol, or about the molecular weight of PFTeDA. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 11. In some embodiments, the fluorinated solvent is perfluorohexane and the perfluorinated dispersant compound comprises Compound 11. In some embodiments, the fluorinated solvent is perfluorooctane and the perfluorinated dispersant compound comprises Compound 11. In some embodiments, the fluorinated solvent is perfluoropentane and the perfluorinated dispersant compound comprises Compound 11. In some embodiments, the fluorinated solvent is perfluoroheptane and the perfluorinated dispersant compound comprises Compound 11. In some embodiments, the fluorinated solvent is perfluoromethylcyclohexane and the perfluorinated dispersant compound comprises Compound 11. In some embodiments, the fluorinated solvent is perfluoro-1-3-dimethylcyclohexane and the perfluorinated dispersant compound comprises Compound 11. In some embodiments, the fluorinated solvent is perfluorodecalin and the perfluorinated dispersant compound comprises Compound 11.

In some embodiments, the protein of interest is dispersed in perfluorohexane in the presence of Compound 11. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 11, wherein the molar ratio of Compound 11 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 11, wherein the molar ratio of Compound 11 is about 1000:1 with the protein of interest. In some embodiments, the protein of interest phase separates when dispersed in perfluorohexane in the presence of Compound 11, but does not phase separate when dispersed in perfluorohexane in the absence of Compound 11. In some embodiments, the protein of interest phase separates when dispersed in perfluorohexane in the presence of Compound 11, wherein the dispersion efficiency is about 5%, 10%, 15%, 20%, 25%, 30%, 35%, 40%, 45%, 50%, 55%, or about 60%. In some embodiments, the dispersion efficiency is between about 5% and about 60%. In some embodiments, the dispersion efficiency is about 50%. In some embodiments, the dispersion efficiency is about 25%. In some embodiments, the dispersion efficiency is about 10%.

In some embodiments, the dispersant compound comprises Compound 12. In some embodiments, Compound 12 comprises perfluorooctadecanoic acid. In some embodiments, Compound 12 comprises the formula C₁₈HF₃₅02. In some embodiments, the dispersant compound comprises Formula II ((CF₂)_{X}(CF₃)₂COOH), wherein x is 16 ((CF₂)₁₆CF₃COOH), and the compound is Compound 12 comprising perfluorooctadecanoic acid. In some embodiments, perfluorooctadecanoic acid is 2,2,3,3,4,4,5,5,6,6,7,7,8,8,9,9,10,10,11,11,12,12,13,13,14,14,15,15,16,16,17,17,18,18,18-pentatriacontafluorooctadecanoic acid. In some embodiments, the molecular weight of Compound 12 is about 914 g/mol, or about the molecular weight of perfluorooctadecanoic acid. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 12. In some embodiments, the fluorinated solvent is perfluorohexane and the perfluorinated dispersant compound comprises Compound 12. In some embodiments, the fluorinated solvent is perfluorooctane and the perfluorinated dispersant compound comprises Compound 12. In some embodiments, the fluorinated solvent is perfluoropentane and the perfluorinated dispersant compound comprises Compound 12. In some embodiments, the fluorinated solvent is perfluoroheptane and the perfluorinated dispersant compound comprises Compound 12. In some embodiments, the fluorinated solvent is perfluoromethylcyclohexane and the perfluorinated dispersant compound comprises Compound 12. In some embodiments, the fluorinated solvent is perfluoro-1-3-dimethylcyclohexane and the perfluorinated dispersant compound comprises Compound 12. In some embodiments, the fluorinated solvent is perfluorodecalin and the perfluorinated dispersant compound comprises Compound 12.

In some embodiments, the protein of interest is dispersed in perfluorohexane in the presence of Compound 12. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 12, wherein the molar ratio of Compound 12 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 12, wherein the molar ratio of Compound 12 is about 1000:1 with the protein of interest. In some embodiments, the protein of interest phase separates when dispersed in perfluorohexane in the presence of Compound 12, but does not phase separate when dispersed in perfluorohexane in the absence of Compound 12. In some embodiments, the protein of interest phase separates when dispersed in perfluorohexane in the presence of Compound 12, wherein the dispersion efficiency is about 5%, 10%, 15%, 20%, or about 25%. In some embodiments, the dispersion efficiency is between about 5% and about 25%. In some embodiments, the dispersion efficiency is about 25%. In some embodiments, the dispersion efficiency is about 10%.

In some embodiments, the dispersant compound comprises Formula III:

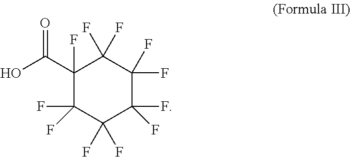 [SMILES: O=C(O)C1(F)C(F)(F)C(F)(F)C(F)(F)C(F)(F)C1(F)F]

In some embodiments, the Formula III comprises the formula C₆F₁₁COOH. In some embodiments, Formula III is perfluorocyclohexanecarboxylic acid. In some embodiments, Compound 13 comprises perfluorocyclohexanecarboxylic acid. In some embodiments, Compound 13 comprises the formula C₇HF₁₁O₂. In some embodiments, the dispersant compound comprises Formula III (C₆F₁₁COOH), and the compound is Compound 13 comprising perfluorocyclohexanecarboxylic acid. In some embodiments, perfluorocyclohexanecarboxylic acid is 1,2,2,3,3,4,4,5,5,6,6-undecafluorocyclohexane-1-carboxylic acid. In some embodiments, the molecular weight of Compound 13 is about 326 g/mol, or about the molecular weight of perfluorocyclohexanecarboxylic acid. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 13. In some embodiments, the fluorinated solvent is perfluorohexane and the perfluorinated dispersant compound comprises Compound 13. In some embodiments, the fluorinated solvent is perfluorooctane and the perfluorinated dispersant compound comprises Compound 13. In some embodiments, the fluorinated solvent is perfluoropentane and the perfluorinated dispersant compound comprises Compound 13. In some embodiments, the fluorinated solvent is perfluoroheptane and the perfluorinated dispersant compound comprises Compound 13. In some embodiments, the fluorinated solvent is perfluoromethylcyclohexane and the perfluorinated dispersant compound comprises Compound 13. In some embodiments, the fluorinated solvent is perfluoro-1-3-dimethylcyclohexane and the perfluorinated dispersant compound comprises Compound 13. In some embodiments, the fluorinated solvent is perfluorodecalin and the perfluorinated dispersant compound comprises Compound 13.

In some embodiments, the protein of interest is dispersed in perfluorohexane in the presence of Compound 13. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 13, wherein the molar ratio of Compound 13 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 13, wherein the molar ratio of Compound 13 is about 1000:1 with the protein of interest. In some embodiments, the protein of interest phase separates when dispersed in perfluorohexane in the presence of Compound 13, but does not phase separate when dispersed in perfluorohexane in the absence of Compound 13. In some embodiments, the protein of interest phase separates when dispersed in perfluorohexane in the presence of Compound 13, wherein the dispersion efficiency is about 5%, 10%, 15%, 20%, 25%, 30%, 35%, 40%, 45%, 50%, 55%, 60%, 65%, 70%, 75% or about 80%. In some embodiments, the dispersion efficiency is between about 5% and about 80%. In some embodiments, the dispersion efficiency is about 80%. In some embodiments, the dispersion efficiency is about 25%. In some embodiments, the dispersion efficiency is about 10%.

In some embodiments, the dispersant compound comprises Formula IV:

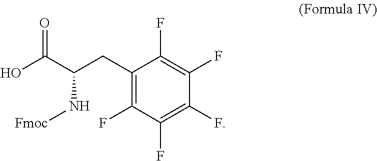

In some embodiments, Formula IV comprises (2S) 2-FMOC-amino-3-pentafluorophenyl propanoic acid. In some embodiments, Formula IV comprises an 9-fluorenylmethyloxycarbonyl (FMOC) protecting group. In some embodiments, Formula IV comprises Fmoc-L-pentafluorophenylalanine. In some embodiments, Formula IV comprises (2S)-2-({[(9H-fluoren-9-yl)methoxy]carbonyl}amino)-3-(2,3,4,5,6-pentafluorophenyl)propanoic acid. In some embodiments, the dispersant compound comprises Compound 14. In some embodiments, Compound 14 comprises (2S)-2-({[(9H-fluoren-9-yl)methoxy]carbonyl}amino)-3-(2,3,4,5,6-pentafluorophenyl)propanoic acid. In some embodiments, Compound 14 comprises the formula C₂₄H₁₆F₅NO₄. In some embodiments, the dispersant compound comprises Formula 14 (C₂₄H₁₆F₅NO₄), wherein and the compound is Compound 14 comprising (2S)-2-({[(9H-fluoren-9-yl)methoxy]carbonyl}amino)-3-(2,3,4,5,6-pentafluorophenyl)propanoic acid. In some embodiments, the molecular weight of Compound 14 is about 477 g/mol, or about the molecular weight of (2S)-2-({[(9H-fluoren-9-yl)methoxy]carbonyl}amino)-3-(2,3,4,5,6-pentafluorophenyl)propanoic acid. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 14. In some embodiments, the fluorinated solvent is perfluorohexane and the perfluorinated dispersant compound comprises Compound 14. In some embodiments, the fluorinated solvent is perfluorooctane and the perfluorinated dispersant compound comprises Compound 14. In some embodiments, the fluorinated solvent is perfluoropentane and the perfluorinated dispersant compound comprises Compound 14. In some embodiments, the fluorinated solvent is perfluoroheptane and the perfluorinated dispersant compound comprises Compound 14. In some embodiments, the fluorinated solvent is perfluoromethylcyclohexane and the perfluorinated dispersant compound comprises Compound 14. In some embodiments, the fluorinated solvent is perfluoro-1-3-dimethylcyclohexane and the perfluorinated dispersant compound comprises Compound 14. In some embodiments, the fluorinated solvent is perfluorodecalin and the perfluorinated dispersant compound comprises Compound 14.

In some embodiments, the protein of interest is dispersed in perfluorohexane in the presence of Compound 14. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 14, wherein the molar ratio of Compound 14 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 14, wherein the molar ratio of Compound 14 is about 1000:1 with the protein of interest. In some embodiments, the protein of interest phase separates when dispersed in perfluorohexane in the presence of Compound 14, but does not phase separate when dispersed in perfluorohexane in the absence of Compound 14. In some embodiments, the protein of interest phase separates when dispersed in perfluorohexane in the presence of Compound 14, wherein the dispersion efficiency is about 1%, 2%, 3%, 4%, 5%, 6%, 7%, 8%, 9%, or 10%. In some embodiments, the dispersion efficiency is between about 1% and about 10%. In some embodiments, the dispersion efficiency is about 5%.

In some embodiments, the dispersant compound comprises Formula V:

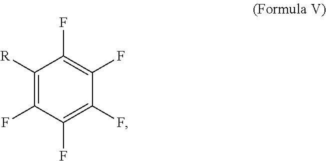

wherein the formula is C₆F₅R and R is OH or COH. In some embodiments, R is OH and the compound is pentafluorophenol. In some embodiments, R is COH and the compound is pentafluorobenzaldehyde.

In some embodiments, the dispersant compound comprises Compound 15. In some embodiments, Compound 15 comprises pentafluorophenol. In some embodiments, Compound 15 comprises the formula C₆HF₅O. In some embodiments, the dispersant compound comprises Formula V (C₆F₅R), where R is OH (C₆F₅OH), and the compound is Compound 15 comprising pentafluorophenol. In some embodiments, pentafluorophenol is 2,3,4,5,6-pentafluorophenol. In some embodiments, the molecular weight of Compound 15 is about 184 g/mol, or about the molecular weight of pentafluorophenol. In some embodiments, the fluorinated solvent is chosen from the group that includes perfluoropentane, perfluorohexane, perfluoroheptane, perfluorooctane, pertluoromethylcyclohexane, perfluoro-1-3-dimethylcyclohexane, or perfluorodecalin, and the perfluorinated dispersant compound comprises Compound 15. In some embodiments, the fluorinated solvent is perfluorohexane and the perfluorinated dispersant compound comprises Compound 15. In some embodiments, the fluorinated solvent is perfluorooctane and the perfluorinated dispersant compound comprises Compound 15. In some embodiments, the fluorinated solvent is perfluoropentane and the perfluorinated dispersant compound comprises Compound 15. In some embodiments, the fluorinated solvent is perfluoroheptane and the perfluorinated dispersant compound comprises Compound 15. In some embodiments, the fluorinated solvent is perfluoromethylcyclohexane and the perfluorinated dispersant compound comprises Compound 15. In some embodiments, the fluorinated solvent is perfluoro-1-3-dimethylcyclohexane and the perfluorinated dispersant compound comprises Compound 15. In some embodiments, the fluorinated solvent is perfluorodecalin and the perfluorinated dispersant compound comprises Compound 15.

In some embodiments, the protein of interest is dispersed in perfluorohexane in the presence of Compound 15. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 15, wherein the molar ratio of Compound 15 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 15, wherein the molar ratio of Compound 15 is about 1000:1 with the protein of interest. In some embodiments, the protein of interest phase separates when dispersed in perfluorohexane in the presence of Compound 15, but does not phase separate when dispersed in perfluorohexane in the absence of Compound 15. In some embodiments, the protein of interest phase separates when dispersed in perfluorohexane in the presence of Compound 15, wherein the dispersion efficiency is about 5%, 10%, 15%, 20%, 25%, 30%, 35%, or about 40%. In some embodiments, the dispersion efficiency is between about 5% and about 40%. In some embodiments, the dispersion efficiency is about 40%. In some embodiments, the dispersion efficiency is about 10%.

In some embodiments, the dispersant compound comprises Compound 16. In some embodiments, Compound 16 comprises pentafluorobenzaldehyde. In some embodiments, Compound 16 comprises the formula C₇HF₅O. In some embodiments, the dispersant compound comprises Formula V (C₆F₅R), where R is COH (C₆F₅COH), and the compound is Compound 16 comprising pentafluorobenzaldehyde. In some embodiments, pentafluorobenzaldehyde is 2,3,4,5,6-pentafluorobenzaldehyde. In some embodiments, the molecular weight of Compound 16 is about 196 g/mol, or about the molecular weight of pentafluorobenzaldehyde. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 16. In some embodiments, the fluorinated solvent is perfluorohexane and the perfluorinated dispersant compound comprises Compound 16. In some embodiments, the fluorinated solvent is perfluorooctane and the perfluorinated dispersant compound comprises Compound 16. In some embodiments, the fluorinated solvent is perfluoropentane and the perfluorinated dispersant compound comprises Compound 16. In some embodiments, the fluorinated solvent is perfluoroheptane and the perfluorinated dispersant compound comprises Compound 16. In some embodiments, the fluorinated solvent is perfluoromethylcyclohexane and the perfluorinated dispersant compound comprises Compound 16. In some embodiments, the fluorinated solvent is perfluoro-1-3-dimethylcyclohexane and the perfluorinated dispersant compound comprises Compound 16. In some embodiments, the fluorinated solvent is perfluorodecalin and the perfluorinated dispersant compound comprises Compound 16.

In some embodiments, the protein of interest is dispersed in perfluorohexane in the presence of Compound 16. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 16, wherein the molar ratio of Compound 16 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 16, wherein the molar ratio of Compound 16 is about 1000:1 with the protein of interest. In some embodiments, the protein of interest phase separates when dispersed in perfluorohexane in the presence of Compound 16, but does not phase separate when dispersed in perfluorohexane in the absence of Compound 16. In some embodiments, the protein of interest phase separates when dispersed in perfluorohexane in the presence of Compound 16, wherein the dispersion efficiency is about 5%, 10%, 15%, 20%, or about 25%. In some embodiments, the dispersion efficiency is between about 5% and about 80%. In some embodiments, the dispersion efficiency is about 10%.

In some embodiments, the dispersant compound comprises Formula VI:

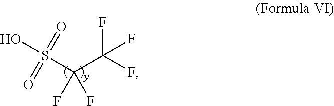

wherein the formula is (CF₂)_{Y}CF₃SO₂OH and y is 7. In some embodiments, the dispersant compound comprises Compound 17. In some embodiments, Compound 17 comprises perfluorooctanesulfonic acid (PFOS). In some embodiments, Compound 17 comprises the formula C₈HF₁₇O₃S. In some embodiments, the dispersant compound comprises Formula VI ((CF₂)_{Y}CF₃SO₂OH), wherein y is 7 ((CF₂)₇CF₃SO₂OH), and the compound is Compound 17 comprising perfluorooctanesulfonic acid. In some embodiments, perfluorooctanesulfonic acid is 1,1,2,2,3,3,4,4,5,5,6,6,7,7,8,8,8-heptadecafluorooctane-1-sulfonic acid. In some embodiments, the molecular weight of Compound 17 is about 500 g/mol, or about the molecular weight of perfluorooctanesulfonic acid. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 17. In some embodiments, the fluorinated solvent is perfluorohexane and the perfluorinated dispersant compound comprises Compound 17. In some embodiments, the fluorinated solvent is perfluorooctane and the perfluorinated dispersant compound comprises Compound 17. In some embodiments, the fluorinated solvent is perfluoropentane and the perfluorinated dispersant compound comprises Compound 17. In some embodiments, the fluorinated solvent is perfluoroheptane and the perfluorinated dispersant compound comprises Compound 17. In some embodiments, the fluorinated solvent is perfluoromethylcyclohexane and the perfluorinated dispersant compound comprises Compound 17. In some embodiments, the fluorinated solvent is perfluoro-1-3-dimethylcyclohexane and the perfluorinated dispersant compound comprises Compound 17. In some embodiments, the fluorinated solvent is perfluorodecalin and the perfluorinated dispersant compound comprises Compound 17.

In some embodiments, the protein of interest is dispersed in perfluorohexane in the presence of Compound 17. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 17, wherein the molar ratio of Compound 17 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 17, wherein the molar ratio of Compound 17 is about 1000:1 with the protein of interest. In some embodiments, the protein of interest phase separates when dispersed in perfluorohexane in the presence of Compound 17, but does not phase separate when dispersed in perfluorohexane in the absence of Compound 17. In some embodiments, the protein of interest phase separates when dispersed in perfluorohexane in the presence of Compound 17, wherein the dispersion efficiency is about 5%, 10%, 15%, 20%, 25%, 30%, 35%, 40%, 45%, 50%, 55%, or about 60%. In some embodiments, the dispersion efficiency is between about 5% and about 60%. In some embodiments, the dispersion efficiency is about 60%. In some embodiments, the dispersion efficiency is about 25%. In some embodiments, the dispersion efficiency is about 10%.

In some embodiments, the dispersion efficiency of the protein of interest dissolved in PFH in the presence of PFOS is at least about 5%, 10%, 20%, 30%, 40%, 50%, 60%, 70%, 80%, or 90% when the protein of interest has a molecular weight of about 5 kDa to about 600 kDa. In some embodiments, the dispersion efficiency of the protein of interest is at least about 1%, 5%, 10%, 20%, 30%, 40%, 50%, 60%, 70%, 80%, or 90% when the protein of interest has a molecular weight of about 5 kDa to about 100 kDa. In some embodiments, the dispersion efficiency of the protein of interest dissolved in PFH in the presence of PFOS is at least about 35% when the protein of interest has a molecular weight of about 5 kDa to about 600 kDa.

In some embodiments, the dispersion efficiency of the protein of interest dissolved in PFH in the presence of PFOS is at least about 5%, 10%, 20%, 30%, 40%, 50%, 60%, 70%, 80%, or 90% when the solvent accessible surface area (SASA) is between about 1×10⁴ Å and about 15×10⁴ Å. In some embodiments, the dispersion efficiency of the protein of interest dissolved in PFH in the presence of PFOS is at least about 5%, 10%, 20%, 30%, 40%, 50%, 60%, 70%, 80%, or 90% when the hydrodynamic radii of the protein of interest is between about 2 and to 8 nm. In some embodiments, the dispersion efficiency of the protein of interest dissolved in PFH in the presence of PFOS is at least about 5%, 10%, 20%, 30%, 40%, 50%, 60%, 70%, 80%, or 90% when the isoelectric point (pI) of the protein of interest is between about 4 to about 8. In some embodiments, the dispersion efficiency of the protein of interest dissolved in PFH in the presence of PFOS is at least about 5%, 10%, 20%, 30%, 40%, 50%, 60%, 70%, 80%, or 90% when the α helicity content of the protein of interest is between 0% and about 35%. In some embodiments, the dispersion efficiency of the protein of interest dissolved in PFH in the presence of PFOS is at least about 5%, 10%, 20%, 30%, 40%, 50%, 60%, 70%, 80%, or 90% when the β sheet content of the protein of interest is between 0% and about 60%.

In some embodiments, the dispersion efficiency of the protein of interest dissolved in PFH in the presence of PFOS is between about 5% to about 100% when the protein of interest contains between about 0% to about 30% polar residues. In some embodiments, the dispersion efficiency of the protein of interest dissolved in PFH in the presence of PFOS is between about 35% to about 100% when the protein of interest contains between about 0% to about 30% polar residues. In some embodiments, the dispersion efficiency of the protein of interest dissolved in PFH in the presence of PFOS is between about 5% to about 100% when the protein of interest contains between about 0% to about 45% nonpolar residues. In some embodiments, the dispersion efficiency of the protein of interest dissolved in PFH in the presence of PFOS is between about 35% to about 100% when the protein of interest contains between about 0% to about 45% nonpolar residues.

In some embodiments, the protein of interest dissolved in PFH in the presence of PFOS, wherein the dispersant compound PFOS interacts with the protein of interest via hydrogen bonding with solvent shell H₂O molecules interacting with the protein of interest. In some embodiments, the protein of interest is dissolved in PFH in the presence of PFOS, wherein the dispersant compound PFOS interacts with the protein of interest via hydrogen bonding with nitrogen donors present in the protein backbone or the amino acid side chains of the protein of interest. In some embodiments, the protein of interest is dissolved in PFH in the presence of PFOS, wherein the dispersant compound PFOS interacts with the protein of interest via hydrogen bonding with oxygen donors present in the protein backbone or the amino acid side chains of the protein of interest. In some embodiments, the protein of interest is dissolved in PFH in the presence of PFOS, wherein the dispersant compound PFOS interacts with the protein of interest via hydrogen bonding with sulfur donors present in the amino acid side chains of the protein of interest. In some embodiments, the protein of interest dissolved in PFH in the presence of PFOS, wherein the carboxylic acid group of the dispersant compound PFOS interacts with the protein of interest via hydrogen bonding to the H₂O molecules interacting with the protein of interest. In some embodiments, the protein of interest dissolved in PFH in the presence of PFOS, wherein the carboxylic acid group of the dispersant compound PFOS interacts with the protein of interest via hydrogen bonding to the nitrogen, oxygen, or sulfur donors in the protein backbone or the amino acid side chains of the protein of interest. In some embodiments, the protein of interest dissolved in PFH in the presence of PFOS, wherein the one or more fluorine group of the dispersant compound PFOS interacts with the protein of interest via hydrogen bonding to the H₂O molecules interacting with the protein of interest. In some embodiments, the protein of interest dissolved in PFH in the presence of PFOS, wherein the one or more fluorine group of the dispersant compound PFOS interacts with the protein of interest via hydrogen bonding to the nitrogen, oxygen, or sulfur donors in the protein backbone or the amino acid side chains of the protein of interest.

In some embodiments, the protein of interest is dissolved in PFH in the presence of PFOS, wherein the dispersant compound PFOS makes hydrophobic contacts with protein solvent accessible amino acids. In some embodiments, the protein of interest is dissolved in PFH in the presence of PFOS, wherein the Gibbs free energy between the PFOS and the protein of interest is between about −20 to about −50 kcal/mol. In some embodiments, the protein of interest is dissolved in PFH in the presence of PFOS, wherein the Gibbs free energy between the PFOS and the docked rotamers from the protein of interest is between about −20 to about −50 kcal/mol.

In some embodiments, the structure of the protein of interest changes when dissolved in PFH in the presence of PFOS. In some embodiments, the protein of interest comprises a non-native secondary structure. In some embodiments, the change in molar ellipticity of the protein of interest is between about 2 deg cm⁻² dmol⁻¹ to about −6 deg cm⁻² dmol⁻¹. In some embodiments, the change in β-sheet content is between about −20% to about 20%. In some embodiments, the change in parallel β-sheet content is between about −20% to about 20%. In some embodiments, the change in antiparallel β-sheet content is between about −20% to about 20%. In some embodiments, the change in α-helix content is between about −20% to about 20%. In some embodiments, the change in turns content is between about −20% to about 20%. In some embodiments, the protein of interest is dissolved in PFH in the presence of PFOS, wherein the binding of the protein to the PFOS is 1:1. In some embodiments, the protein of interest is dissolved in PFH in the presence of PFOS, wherein the binding of the protein to the PFOS is not 1:1.

In some embodiments, the protein of interest is dissolved in PFH in the presence of PFOS, wherein the thermal stability of the protein is increased compared to the thermal stability of the protein of interest dissolved in an aqueous solvent. In some embodiments, the protein of interest is dissolved in PFH in the presence of PFOS, wherein the melting temperature of the protein is increased by about 5 °C, 10 °C, 20 °C, 30 °C, or 40 °C compared to the melting temperature of the protein of interest in an aqueous solvent. In some embodiments, the protein of interest is active in an aqueous solvent at 25 °C, but is not active in an aqueous solvent at 75 °C, 80 °C, 85 °C, or 90 °C. In some embodiments, the protein of interest is active in an aqueous solvent at 25 °C, but the activity is reduced when the protein of interest is dissolved in an aqueous solvent at 75 °C, 80 °C, 85 °C, or 90 °C. In some embodiments, the activity is reduced by about 80% or by about 90%. In some embodiments, the protein of interest is dissolved in PFH in the presence of PFOS, wherein the protein of interest is active at 25 °C and is also active at 75 °C, 80 °C, 85 °C, or 90 °C. In some embodiments, the protein of interest is dissolved in PFH in the presence of PFOS, wherein the activity is reduced by about 1% to about 25% when the temperature is about 75 °C, 80 °C, 85 °C, or 90 °C, compared to when the temperature is about 25 °C. In some embodiments, the protein of interest is dissolved in PFH in the presence of PFOS, wherein the activity is increased by about 1% to about 25% when the temperature is about 75 °C, 80 °C, 85 °C, or 90 °C, compared to when the temperature is about 25 °C.

In some embodiments, the protein of interest is dissolved in PFH in the presence of PFOS, wherein the protein comprises a reduced risk of contamination compared to when the protein is dissolved in an aqueous solvent. In some embodiments, the protein of interest is dissolved in PFH in the presence of PFOS, wherein the protein comprises a reduced risk of bacterial or viral contamination compared to when the protein is dissolved in PBS. In some embodiments, the protein of interest is dissolved in PFH in the presence of PFOS, wherein the protein comprises a reduced risk of E. coli contamination compared to when the protein is dissolved in PBS.

In some embodiments, the dispersant compound comprises Formula VII:

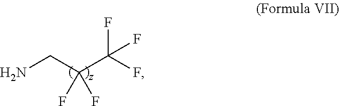

wherein the formula is NH₂CH₂(CF₂)_{Z}CF₃ and z is 2, 6, or 7. In some embodiments, z is 2 (1H,1H-heptafluorobutylamine). In some embodiments, z is 6 (1H,1H-pentadecafluorooctylamine). In some embodiments, z is 7 (1H,1H-perfluorononylamine).

In some embodiments, the dispersant compound comprises Compound 18. In some embodiments, Compound 18 comprises 1H,1H-heptafluorobutylamine. In some embodiments, Compound 18 comprises the formula C₄H₄F₇N. In some embodiments, the dispersant compound comprises Formula VII (NH₂CH₂(CF₂)_{Z}CF₃), wherein z is 2 (NH₂CH₂(CF₂)₂CF₃), and the compound is Compound 18 comprising 1H,1H-heptafluorobutylamine. In some embodiments, 1H,1H-heptafluorobutylamine is 2,2,3,3,4,4,4-heptafluorobutylamine. In some embodiments, the molecular weight of Compound 18 is about 199 g/mol, or about the molecular weight of 1H,1H-heptafluorobutylamine. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 18. In some embodiments, the fluorinated solvent is perfluorohexane and the perfluorinated dispersant compound comprises Compound 18. In some embodiments, the fluorinated solvent is perfluorooctane and the perfluorinated dispersant compound comprises Compound 18. In some embodiments, the fluorinated solvent is perfluoropentane and the perfluorinated dispersant compound comprises Compound 18. In some embodiments, the fluorinated solvent is perfluoroheptane and the perfluorinated dispersant compound comprises Compound 18. In some embodiments, the fluorinated solvent is perfluoromethylcyclohexane and the perfluorinated dispersant compound comprises Compound 18. In some embodiments, the fluorinated solvent is perfluoro-1-3-dimethylcyclohexane and the perfluorinated dispersant compound comprises Compound 18. In some embodiments, the fluorinated solvent is perfluorodecalin and the perfluorinated dispersant compound comprises Compound 18.

In some embodiments, the protein of interest is dispersed in perfluorohexane in the presence of Compound 18. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 18, wherein the molar ratio of Compound 18 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 18, wherein the molar ratio of Compound 18 is about 1000:1 with the protein of interest. In some embodiments, the protein of interest phase separates when dispersed in perfluorohexane in the presence of Compound 18, but does not phase separate when dispersed in perfluorohexane in the absence of Compound 18. In some embodiments, the protein of interest phase separates when dispersed in perfluorohexane in the presence of Compound 18, wherein the dispersion efficiency is about 5%, 10%, 15%, 20%, 25%, 30%, or about 35%. In some embodiments, the dispersion efficiency is between about 5% and about 35%. In some embodiments, the dispersion efficiency is about 80%. In some embodiments, the dispersion efficiency is about 25%. In some embodiments, the dispersion efficiency is about 10%.

In some embodiments, the dispersant compound comprises Compound 19. In some embodiments, Compound 19 comprises 1H,1H-pentadecafluorooctylamine. In some embodiments, Compound 19 comprises the formula C₈H₄F₁₅N. In some embodiments, the dispersant compound comprises Formula VII (NH₂CH₂(CF₂)_{Z}CF₃), wherein z is 6 (NH₂CH₂(CF₂)₆CF₃), and the compound is Compound 19 comprising 1H,1H-pentadecafluorooctylamine. In some embodiments, 1H,1H-pentadecafluorooctylamine is 2,2,3,3,4,4,5,5,6,6,7,7,8,8,8-pentadecafluorooctan-1-amine. In some embodiments, the molecular weight of Compound 19 is about 399 g/mol, or about the molecular weight of 1H,1H-pentadecafluorooctylamine. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 19. In some embodiments, the fluorinated solvent is perfluorohexane and the perfluorinated dispersant compound comprises Compound 19. In some embodiments, the fluorinated solvent is perfluorooctane and the perfluorinated dispersant compound comprises Compound 19. In some embodiments, the fluorinated solvent is perfluoropentane and the perfluorinated dispersant compound comprises Compound 19. In some embodiments, the fluorinated solvent is perfluoroheptane and the perfluorinated dispersant compound comprises Compound 19. In some embodiments, the fluorinated solvent is perfluoromethylcyclohexane and the perfluorinated dispersant compound comprises Compound 19. In some embodiments, the fluorinated solvent is perfluoro-1-3-dimethylcyclohexane and the perfluorinated dispersant compound comprises Compound 19. In some embodiments, the fluorinated solvent is perfluorodecalin and the perfluorinated dispersant compound comprises Compound 19.

In some embodiments, the protein of interest is dispersed in perfluorohexane in the presence of Compound 19. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 19, wherein the molar ratio of Compound 19 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 19, wherein the molar ratio of Compound 19 is about 1000:1 with the protein of interest. In some embodiments, the protein of interest phase separates when dispersed in perfluorohexane in the presence of Compound 19, but does not phase separate when dispersed in perfluorohexane in the absence of Compound 19. In some embodiments, the protein of interest phase separates when dispersed in perfluorohexane in the presence of Compound 19, wherein the dispersion efficiency is about 5%, 10%, 15%, 20%, or about 25%. In some embodiments, the dispersion efficiency is between about 5% and about 25%. In some embodiments, the dispersion efficiency is about 10%.

In some embodiments, the dispersant compound comprises Compound 20. In some embodiments, Compound 20 comprises 1H,1H-perfluorononylamine. In some embodiments, Compound 20 comprises the formula C₉H₄F₁₇N. In some embodiments, the dispersant compound comprises Formula VII (NH₂CH₂(CF₂)_{Z}CF₃), wherein z is 7 (NH₂CH₂(CF₂)₇CF₃), and the compound is Compound 20 comprising 1H,1H-perfluorononylamine. In some embodiments, 1H,1H-perfluorononylamine is 2,2,3,3,4,4,5,5,6,6,7,7,8,8,9,9,9-heptadecafluorononan-1-amine. In some embodiments, the molecular weight of Compound 20 is about 449 g/mol, or about the molecular weight of 1H,1H-perfluorononylamine. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 20. In some embodiments, the fluorinated solvent is perfluorohexane and the perfluorinated dispersant compound comprises Compound 20. In some embodiments, the fluorinated solvent is perfluorooctane and the perfluorinated dispersant compound comprises Compound 20. In some embodiments, the fluorinated solvent is perfluoropentane and the perfluorinated dispersant compound comprises Compound 20. In some embodiments, the fluorinated solvent is perfluoroheptane and the perfluorinated dispersant compound comprises Compound 20. In some embodiments, the fluorinated solvent is perfluoromethylcyclohexane and the perfluorinated dispersant compound comprises Compound 20. In some embodiments, the fluorinated solvent is perfluoro-1-3-dimethylcyclohexane and the perfluorinated dispersant compound comprises Compound 20. In some embodiments, the fluorinated solvent is perfluorodecalin and the perfluorinated dispersant compound comprises Compound 20.

In some embodiments, the protein of interest is dispersed in perfluorohexane in the presence of Compound 20. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 20, wherein the molar ratio of Compound 20 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 20, wherein the molar ratio of Compound 20 is about 1000:1 with the protein of interest. In some embodiments, the protein of interest phase separates when dispersed in perfluorohexane in the presence of Compound 20, but does not phase separate when dispersed in perfluorohexane in the absence of Compound 20. In some embodiments, the protein of interest phase separates when dispersed in perfluorohexane in the presence of Compound 20, wherein the dispersion efficiency is about 5%, 10%, 15%, 20%, 25%, 30%, 35%, 40%, 45%, 50%, 55%, or about 60%. In some embodiments, the dispersion efficiency is between about 5% and about 60%. In some embodiments, the dispersion efficiency is about 50%. In some embodiments, the dispersion efficiency is about 25%. In some embodiments, the dispersion efficiency is about 10%.

In some embodiments, the dispersant compound comprises Formula VIII:

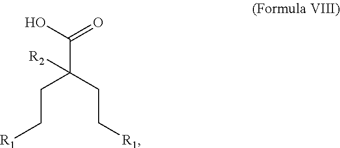

wherein the formula is C₅H₈(R1)₂(R2)COOH, wherein R₁ is C₆F₁₃, C₈F₁₇, or C₄F₉, and wherein R₂ is CN, H, or COOH. In some embodiments, the dispersant compound based on Formula VIII is chosen from: 2,2-(1,1,1,2,2,3,3,4,4,5,5,6,6-tridecafluorooctane)-2-cyanoacetic acid, 2,2-(1,1,1,2,2,3,3,4,4,5,5,6,6-tridecafluorooctane)-2-acetic acid, 2,2-(1,1,1,2,2,3,3,4,4,5,5,6,6,7,7,8,8-heptadecafluorodecane)-2-cyanoacetic acid, 2,2-(1,1,1,2,2,3,3,4,4,5,5,6,6,7,7,8,8-heptadecafluorodecane)-acetic acid, 2,2-(1,1,1,2,2,3,3,4,4-nonafluorohexane)-2-cyanoacetic acid, 2,2-(1,1,1,2,2,3,3,4,4-nonafluorohexane)-2-acetic acid, 2,2-(1,1,1,2,2,3,3,4,4-nonafluorohexane)-2-diacetic acid, 2,2-(1,1,1,2,2,3,3,4,4,5,5,6,6-tridecafluorooctane)-2-diacetic acid, or 2,2-(1,1,1,2,2,3,3,4,4,5,5,6,6,7,7,8,8-heptadecafluorodecane)-2-diacetic acid.

In some embodiments, the dispersant compound comprises Compound 21, wherein the formula is based on Formula VIII, and wherein R₁ is C₆F₁₃ and R₂ is CN. In some embodiments, the dispersant compound comprises Compound 22, wherein the formula is based on Formula VIII, and wherein R₁ is C₆F₁₃ and R₂ is H. In some embodiments, the dispersant compound comprises Compound 23, wherein the formula is based on Formula VIII, and wherein R₁ is C₈F₁₇ and R₂ is CN. In some embodiments, the dispersant compound comprises Compound 24, wherein the formula is based on Formula VIII, and wherein R₁ is C₈F₁₇ and R₂ is CN. In some embodiments, the dispersant compound comprises Compound 25, wherein the formula is based on Formula VIII, and wherein R₁ is C₄F₉ and R₂ is CN. In some embodiments, the dispersant compound comprises Compound 26, wherein the formula is based on Formula VIII, and wherein R₁ is C₄F₉ and R₂ is H. In some embodiments, the dispersant compound comprises Compound 27, wherein the formula is based on Formula VIII, and wherein R₁ is C₄F₉ and R₂ is COOH. In some embodiments, the dispersant compound comprises Compound 28, wherein the formula is based on Formula VIII, and wherein R₁ is C₆F₁₃ and R₂ is COOH. In some embodiments, the dispersant compound comprises Compound 29, wherein the formula is based on Formula VIII, and wherein R₁ is C₈F₁₇ and R₂ is COOH.

In some embodiments, the perfluorinated dispersant compound comprises a chemical name chosen from the group described in Table 3, where R1_Extended comprises the chemical group R1_Extended chosen from the group described in Table 4. In some embodiments, the dispersant compound comprises Formula VIII_B:

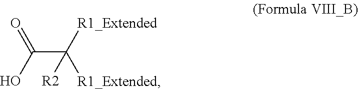

wherein the formula is C(R1_Extended)₂(R2)COOH, wherein R₁_Extended is C₈H₅F₁₃, C₁₀H₅F₁₇, or C₆H₅F₉, and wherein R₂ is CN, H, or COOH. In some embodiments, the dispersant compound comprises Compound 21, wherein the formula is based on Formula VIII_B, and wherein R₁_Extended is C₈H₅F₁₃ and R₂ is CN. In some embodiments, the dispersant compound comprises Compound 22, wherein the formula is based on Formula VIII_B, and wherein R₁_Extended is C₈H₅F₁₃ and R₂ is H. In some embodiments, the dispersant compound comprises Compound 23, wherein the formula is based on Formula VIII_B, and wherein R₁_Extended is C₁₀H₅F₁₇ and R₂ is CN. In some embodiments, the dispersant compound comprises Compound 24, wherein the formula is based on Formula VIII_B, and wherein R₁_Extended is C₁₀H₅F₁₇ and R₂ is CN. In some embodiments, the dispersant compound comprises Compound 25, wherein the formula is based on Formula VIII_B, and wherein R₁_Extended is C₆H₅F₉ and R₂ is CN. In some embodiments, the dispersant compound comprises Compound 26, wherein the formula is based on Formula VIII_B, and wherein R₁_Extended is C₆H₅F₉ and R₂ is H. In some embodiments, the dispersant compound comprises Compound 27, wherein the formula is based on Formula VIII_B, and wherein R₁_Extended is C₆H₅F₉ and R₂ is COOH. In some embodiments, the dispersant compound comprises Compound 28, wherein the formula is based on Formula VIII_B, and wherein R₁_Extended is C₈H₅F₁₃ and R₂ is COOH. In some embodiments, the dispersant compound comprises Compound 29, wherein the formula is based on Formula VIII_B, and wherein R₁_Extended is C₁₀H₅F₁₇ and R₂ is COOH

In some embodiments, the Compound 21 comprises a chemical group R1, wherein R1 is C₆F₁₃. In some embodiments, the Compound 21 comprises two chemical groups R1, wherein R1 is C₆F₁₃. In some embodiments, the Compound 21 comprises the group R2, wherein R₂ is CN. In some embodiments, the Compound 21 comprises two chemical groups R1, wherein R1 is C₆F₁₃, and additionally comprises the group R₂, wherein R₂ is CN. In some embodiments, Compound 21 comprises Formula VIII:

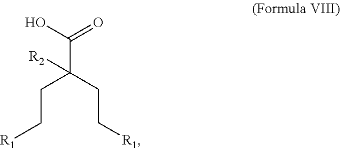

wherein the formula is C₅H₈(R1)₂(R2)COOH, wherein R₁ is C₆F₁₃, and wherein R₂ is CN. In some embodiments, the Compound 21 comprises a chemical group R1_Extended, wherein R1_Extended is C₈H₅F₁₃. In some embodiments, the Compound 21 comprises two chemical groups R1_Extended, wherein R1_Extended is C₈H₅F₁₃. In some embodiments, the Compound 21 comprises two chemical groups R1_Extended, wherein R1_Extended is C₈H₅F₁₃, and additionally comprises the group R₂, wherein R₂ is CN. In some embodiments, Compound 21 comprises Formula VIII_B:

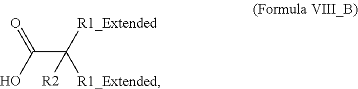

wherein the formula is C(R1_Extended)₂(R2)COOH, wherein R₁_Extended is C₈H₅F₁₃ and wherein R₂ is CN. In some embodiments, Compound 21 comprises the chemical formula 2,2-(R1_Extended)-2-cyanoacetic acid, wherein R1_Extended is the chemical group 1,1,1,2,2,3,3,4,4,5,5,6,6-tridecafluorooctane. In some embodiments, Compound 21 comprises 2,2-(1,1,1,2,2,3,3,4,4,5,5,6,6-tridecafluorooctane)-2-cyanoacetic acid.

In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 21. In some embodiments, the fluorinated solvent is perfluorohexane and the perfluorinated dispersant compound comprises Compound 21. In some embodiments, the fluorinated solvent is perfluorooctane and the perfluorinated dispersant compound comprises Compound 21. In some embodiments, the fluorinated solvent is perfluoropentane and the perfluorinated dispersant compound comprises Compound 21. In some embodiments, the fluorinated solvent is perfluoroheptane and the perfluorinated dispersant compound comprises Compound 21. In some embodiments, the fluorinated solvent is perfluoromethylcyclohexane and the perfluorinated dispersant compound comprises Compound 21. In some embodiments, the fluorinated solvent is perfluoro-1-3-dimethylcyclohexane and the perfluorinated dispersant compound comprises Compound 21. In some embodiments, the fluorinated solvent is perfluorodecalin and the perfluorinated dispersant compound comprises Compound 21.

In some embodiments, the protein of interest is dispersed in perfluorohexane in the presence of Compound 21. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 21, wherein the molar ratio of Compound 21 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 21, wherein the molar ratio of Compound 21 is about 1000:1 with the protein of interest. In some embodiments, the protein of interest phase separates when dispersed in perfluorohexane in the presence of Compound 21, but does not phase separate when dispersed in perfluorohexane in the absence of Compound 21. In some embodiments, the protein of interest phase separates when dispersed in perfluorohexane in the presence of Compound 21, wherein the dispersion efficiency is about 5%, 10%, 15%, 20%, 25%, 30%, 35%, 40%, 45%, 50%, 55%, 60%, 65%, 70%, 75%, 80%, 85%, 90%, 95%, or about 100%. In some embodiments, the dispersion efficiency is between about 5% and about 100%. In some embodiments, the dispersion efficiency is about 100%. In some embodiments, the dispersion efficiency is at least about 50%. In some embodiments, the dispersion efficiency is at least about 40%. In some embodiments, the dispersion efficiency is at least about 25%.

In some embodiments, the dispersant compound comprises Compound 22. In some embodiments, the Compound 22 comprises a chemical group R1, wherein R1 is C₆F₁₃. In some embodiments, the Compound 22 comprises two chemical groups R1, wherein R1 is C₆F₁₃. In some embodiments, the Compound 22 comprises the group R₂, wherein R₂ is CN. In some embodiments, the Compound 22 comprises two chemical groups R1, wherein R1 is C₆F₁₃, and additionally comprises the group R₂, wherein R₂ is H. In some embodiments, Compound 22 comprises Formula VIII:

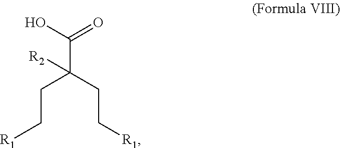

wherein the formula is C₅H₈(R1)₂(R2)COOH, wherein R₁ is C₆F₁₃, and wherein R₂ is H. In some embodiments, the Compound 22 comprises a chemical group R1_Extended, wherein R1_Extended is C₈H₅F₁₃. In some embodiments, the Compound 22 comprises two chemical groups R1_Extended, wherein R1_Extended is C₈H₅F₁₃. In some embodiments, the Compound 22 comprises two chemical groups R1_Extended, wherein R1_Extended is C₈H₅F₁₃, and additionally comprises the group R₂, wherein R₂ is H. In some embodiments, Compound 22 comprises Formula VIII_B:

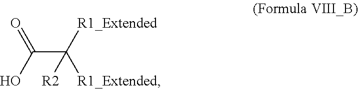

wherein the formula is C(R1_Extended)₂(R2)COOH, wherein R₁_Extended is C₈H₅F₁₃ and wherein R₂ is H. In some embodiments, Compound 22 comprises the chemical formula 2,2-(R1_Extended)-2-acetic acid, wherein R1_Extended is the chemical group 1,1,1,2,2,3,3,4,4,5,5,6,6-tridecafluorooctane. In some embodiments, Compound 22 comprises 2,2-(1,1,1,2,2,3,3,4,4,5,5,6,6-tridecafluorooctane)-2-acetic acid.

In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 22. In some embodiments, the fluorinated solvent is perfluorohexane and the perfluorinated dispersant compound comprises Compound 22. In some embodiments, the fluorinated solvent is perfluorooctane and the perfluorinated dispersant compound comprises Compound 22. In some embodiments, the fluorinated solvent is perfluoropentane and the perfluorinated dispersant compound comprises Compound 22. In some embodiments, the fluorinated solvent is perfluoroheptane and the perfluorinated dispersant compound comprises Compound 22. In some embodiments, the fluorinated solvent is perfluoromethylcyclohexane and the perfluorinated dispersant compound comprises Compound 22. In some embodiments, the fluorinated solvent is perfluoro-1-3-dimethylcyclohexane and the perfluorinated dispersant compound comprises Compound 22. In some embodiments, the fluorinated solvent is perfluorodecalin and the perfluorinated dispersant compound comprises Compound 22.

In some embodiments, the fluorinated solvent is perfluorohexane and the dispersant compound is perfluoronanonic acid. In some embodiments, the fluorinated solvent is perfluorooctane and the dispersant compound is perfluoronanonic acid. In some embodiments, the fluorinated solvent is perfluorohexane and the dispersant compound comprises Formula VIII:

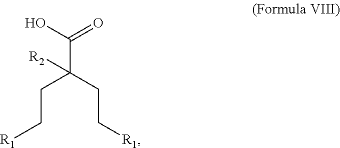

wherein R₁ is C₆F₁₃ and R₂ is H, and the compound is Compound 22. In some embodiments, the fluorinated solvent is perfluorooctane and the dispersant compound comprises Formula VIII:

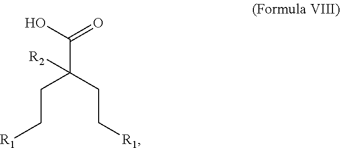

wherein R₁ is C₆F₁₃ and R₂ is H, and the compound is Compound 22.

In some embodiments, the protein of interest is dispersed in perfluorohexane in the presence of Compound 22. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 22, wherein the molar ratio of Compound 22 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 22, wherein the molar ratio of Compound 22 is about 1000:1 with the protein of interest. In some embodiments, the protein of interest phase separates when dispersed in perfluorohexane in the presence of Compound 22, but does not phase separate when dispersed in perfluorohexane in the absence of Compound 22. In some embodiments, the protein of interest phase separates when dispersed in perfluorohexane in the presence of Compound 22, wherein the dispersion efficiency is about 5%, 10%, 15%, 20%, 25%, 30%, 35%, 40%, 45%, 50%, 55%, 60%, 65%, 70%, 75%, 80%, 85%, 90%, 95%, or about 100%. In some embodiments, the dispersion efficiency is between about 5% and about 100%. In some embodiments, the dispersion efficiency is about 100%. In some embodiments, the dispersion efficiency is at least about 90%. In some embodiments, the dispersion efficiency is at least about 50%. In some embodiments, the dispersion efficiency is at least about 25%.

In some embodiments, the dispersant compound comprises Compound 23. In some embodiments, the Compound 23 comprises a chemical group R1, wherein R1 is C₈F₁₇. In some embodiments, the Compound 23 comprises two chemical groups R1, wherein R1 is C₈F₁₇. In some embodiments, the Compound 23 comprises the group R₂, wherein R₂ is CN. In some embodiments, the Compound 23 comprises two chemical groups R1, wherein R1 is C₈F₁₇, and additionally comprises the group R₂, wherein R₂ is CN. In some embodiments, Compound 23 comprises Formula VIII:

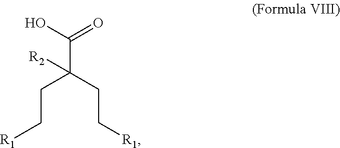

wherein the formula is C₅H₈(R1)₂(R2)COOH, wherein R₁ is C₈F₁₇, and wherein R₂ is CN. In some embodiments, the Compound 23 comprises a chemical group R1_Extended, wherein R1_Extended is C₁₀H₅F₁₇. In some embodiments, the Compound 23 comprises two chemical groups R1 Extended, wherein R1_Extended is C₁₀H₅F₁₇. In some embodiments, the Compound 23 comprises two chemical groups R1_Extended, wherein R1_Extended is C₁₀H₅F₁₇, and additionally comprises the group R₂, wherein R₂ is CN. In some embodiments, Compound 23 comprises Formula VIII_B:

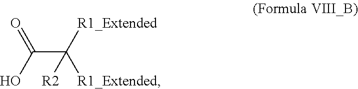

wherein the formula is C(R1_Extended)₂(R2)COOH, wherein R₁_Extended is C₁₀H₅F₁₇ and wherein R₂ is CN. In some embodiments, Compound 23 comprises the chemical formula 2,2-(R1_Extended)-2-cyanoacetic acid, wherein R1_Extended is the chemical group 1,1,1,2,2,3,3,4,4,5,5,6,6,7,7,8,8-heptadecafluorodecane. In some embodiments, Compound 23 comprises 2,2-(1,1,1,2,2,3,3,4,4,5,5,6,6,7,7,8,8-heptadecafluorodecane)-2-cyanoacetic acid.

In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 23. In some embodiments, the fluorinated solvent is perfluorohexane and the perfluorinated dispersant compound comprises Compound 23. In some embodiments, the fluorinated solvent is perfluorooctane and the perfluorinated dispersant compound comprises Compound 23. In some embodiments, the fluorinated solvent is perfluoropentane and the perfluorinated dispersant compound comprises Compound 23. In some embodiments, the fluorinated solvent is perfluoroheptane and the perfluorinated dispersant compound comprises Compound 23. In some embodiments, the fluorinated solvent is perfluoromethylcyclohexane and the perfluorinated dispersant compound comprises Compound 23. In some embodiments, the fluorinated solvent is perfluoro-1-3-dimethylcyclohexane and the perfluorinated dispersant compound comprises Compound 23. In some embodiments, the fluorinated solvent is perfluorodecalin and the perfluorinated dispersant compound comprises Compound 23.

In some embodiments, the protein of interest is dispersed in perfluorohexane in the presence of Compound 23. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 23, wherein the molar ratio of Compound 23 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 23, wherein the molar ratio of Compound 23 is about 1000:1 with the protein of interest. In some embodiments, the protein of interest phase separates when dispersed in perfluorohexane in the presence of Compound 23, but does not phase separate when dispersed in perfluorohexane in the absence of Compound 23. In some embodiments, the protein of interest phase separates when dispersed in perfluorohexane in the presence of Compound 23, wherein the dispersion efficiency is about 5%, 10%, 15%, 20%, 25%, 30%, 35%, 40%, 45%, 50%, 55%, 60%, 65%, 70%, 75%, 80%, 85%, 90%, 95%, or about 100%. In some embodiments, the dispersion efficiency is between about 5% and about 100%. In some embodiments, the dispersion efficiency is about 100%. In some embodiments, the dispersion efficiency is at least about 80%. In some embodiments, the dispersion efficiency is at least about 50%. In some embodiments, the dispersion efficiency is at least about 25%. In some embodiments, the dispersion efficiency is at least about 15%.

In some embodiments, the dispersant compound comprises Compound 24. In some embodiments, the Compound 24 comprises a chemical group R1, wherein R1 is C₈F₁₇. In some embodiments, the Compound 24 comprises two chemical groups R1, wherein R1 is C₈F₁₇. In some embodiments, the Compound 24 comprises the group R₂, wherein R₂ is CN. In some embodiments, the Compound 24 comprises two chemical groups R1, wherein R1 is C₈F₁₇, and additionally comprises the group R₂, wherein R₂ is H. In some embodiments, Compound 24 comprises Formula VIII:

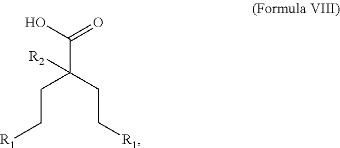

wherein the formula is C₅H₈(R1)₂(R2)COOH, wherein R₁ is C₈F₁₇, and wherein R₂ is H. In some embodiments, the Compound 24 comprises a chemical group R1_Extended, wherein R1_Extended is C₁₀H₅F₁₇. In some embodiments, the Compound 24 comprises two chemical groups R1 Extended, wherein R1_Extended is C₁₀H₅F₁₇. In some embodiments, the Compound 24 comprises two chemical groups R1_Extended, wherein R1_Extended is C₁₀H₅F₁₇, and additionally comprises the group R₂, wherein R₂ is H. In some embodiments, Compound 24 comprises Formula VIII_B:

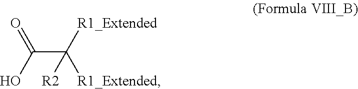

wherein the formula is C(R1_Extended)₂(R2)COOH, wherein R₁_Extended is C₁₀H₅F₁₇ and wherein R₂ is H. In some embodiments, Compound 24 comprises the chemical formula 2,2-(R1_Extended)-2-acetic acid, wherein R1_Extended is the chemical group 1,1,1,2,2,3,3,4,4,5,5,6,6,7,7,8,8-heptadecafluorodecane. In some embodiments, Compound 24 comprises 2,2-(1,1,1,2,2,3,3,4,4,5,5,6,6,7,7,8,8-heptadecafluorodecane)-acetic acid.

In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 24. In some embodiments, the fluorinated solvent is perfluorohexane and the perfluorinated dispersant compound comprises Compound 24. In some embodiments, the fluorinated solvent is perfluorooctane and the perfluorinated dispersant compound comprises Compound 24. In some embodiments, the fluorinated solvent is perfluoropentane and the perfluorinated dispersant compound comprises Compound 24. In some embodiments, the fluorinated solvent is perfluoroheptane and the perfluorinated dispersant compound comprises Compound 24. In some embodiments, the fluorinated solvent is perfluoromethylcyclohexane and the perfluorinated dispersant compound comprises Compound 24. In some embodiments, the fluorinated solvent is perfluoro-1-3-dimethylcyclohexane and the perfluorinated dispersant compound comprises Compound 24. In some embodiments, the fluorinated solvent is perfluorodecalin and the perfluorinated dispersant compound comprises Compound 24.

In some embodiments, the protein of interest is dispersed in perfluorohexane in the presence of Compound 24. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 24, wherein the molar ratio of Compound 24 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 24, wherein the molar ratio of Compound 24 is about 1000:1 with the protein of interest. In some embodiments, the protein of interest phase separates when dispersed in perfluorohexane in the presence of Compound 24, but does not phase separate when dispersed in perfluorohexane in the absence of Compound 24. In some embodiments, the protein of interest phase separates when dispersed in perfluorohexane in the presence of Compound 24, wherein the dispersion efficiency is about 5%, 10%, 15%, 20%, 25%, 30%, 35%, 40%, 45%, 50%, 55%, 60%, 65%, 70%, 75%, 80%, 85%, 90%, 95%, or about 100%. In some embodiments, the dispersion efficiency is between about 5% and about 100%. In some embodiments, the dispersion efficiency is about 100%. In some embodiments, the dispersion efficiency is at least about 80%. In some embodiments, the dispersion efficiency is at least about 50%. In some embodiments, the dispersion efficiency is at least about 25%. In some embodiments, the dispersion efficiency is at least about 15%.

In some embodiments, the dispersant compound comprises Compound 25. In some embodiments, the Compound 25 comprises a chemical group R1, wherein R1 is C₄F₉. In some embodiments, the Compound 25 comprises two chemical groups R1, wherein R1 is C₄F₉. In some embodiments, the Compound 25 comprises the group R₂, wherein R₂ is CN. In some embodiments, the Compound 25 comprises two chemical groups R1, wherein R1 is C₄F₉, and additionally comprises the group R₂, wherein R₂ is CN. In some embodiments, Compound 25 comprises Formula VIII:

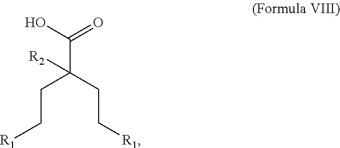

wherein the formula is C₅H₈(R1)₂(R2)COOH, wherein R₁ is C₄F₉, and wherein R₂ is CN. In some embodiments, the Compound 25 comprises a chemical group R1_Extended, wherein R1_Extended is C₆H₅F₉. In some embodiments, the Compound 25 comprises two chemical groups R1_Extended, wherein R1_Extended is C₆H₅F₉. In some embodiments, the Compound 25 comprises two chemical groups R1_Extended, wherein R1_Extended is C₆H₅F₉, and additionally comprises the group R₂, wherein R₂ is CN. In some embodiments, Compound 25 comprises Formula VIII_B:

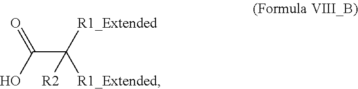

wherein the formula is C(R1_Extended)₂(R2)COOH, wherein R₁_Extended is C₆H₅F₉ and wherein R₂ is CN. In some embodiments, Compound 25 comprises the chemical formula 2,2-(R1_Extended)-2-cyanoacetic acid, wherein R1_Extended is the chemical group 1,1,1,2,2,3,3,4,4-nonafluorohexane. In some embodiments, Compound 25 comprises 2,2-(1,1,1,2,2,3,3,4,4-nonafluorohexane)-2-cyanoacetic acid.

In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 25. In some embodiments, the fluorinated solvent is perfluorohexane and the perfluorinated dispersant compound comprises Compound 25. In some embodiments, the fluorinated solvent is perfluorooctane and the perfluorinated dispersant compound comprises Compound 25. In some embodiments, the fluorinated solvent is perfluoropentane and the perfluorinated dispersant compound comprises Compound 25. In some embodiments, the fluorinated solvent is perfluoroheptane and the perfluorinated dispersant compound comprises Compound 25. In some embodiments, the fluorinated solvent is perfluoromethylcyclohexane and the perfluorinated dispersant compound comprises Compound 25. In some embodiments, the fluorinated solvent is perfluoro-1-3-dimethylcyclohexane and the perfluorinated dispersant compound comprises Compound 25. In some embodiments, the fluorinated solvent is perfluorodecalin and the perfluorinated dispersant compound comprises Compound 25.

In some embodiments, the protein of interest is dispersed in perfluorohexane in the presence of Compound 25. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 25, wherein the molar ratio of Compound 25 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 25, wherein the molar ratio of Compound 25 is about 1000:1 with the protein of interest. In some embodiments, the protein of interest phase separates when dispersed in perfluorohexane in the presence of Compound 25, but does not phase separate when dispersed in perfluorohexane in the absence of Compound 25. In some embodiments, the protein of interest phase separates when dispersed in perfluorohexane in the presence of Compound 25, wherein the dispersion efficiency is about 5%, 10%, 15%, 20%, 25%, 30%, 35%, 40%, 45%, 50%, 55%, 60%, 65%, 70%, 75%, 80%, 85%, 90%, 95%, or about 100%. In some embodiments, the dispersion efficiency is between about 5% and about 100%. In some embodiments, the dispersion efficiency is about 100%. In some embodiments, the dispersion efficiency is at least about 80%. In some embodiments, the dispersion efficiency is at least about 50%. In some embodiments, the dispersion efficiency is at least about 25%. In some embodiments, the dispersion efficiency is at least about 15%.

In some embodiments, the dispersant compound comprises Compound 26. In some embodiments, the Compound 26 comprises a chemical group R1, wherein R1 is C₄F₉. In some embodiments, the Compound 26 comprises two chemical groups R1, wherein R1 is C₄F₉. In some embodiments, the Compound 26 comprises the group R₂, wherein R₂ is CN. In some embodiments, the Compound 26 comprises two chemical groups R1, wherein R1 is C₄F₉, and additionally comprises the group R2, wherein R2 is H. In some embodiments, Compound 26 comprises Formula VIII:

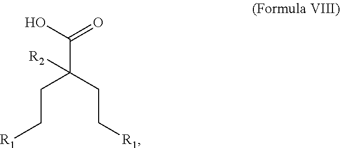

wherein the formula is C₅H₈(R1)₂(R2)COOH, wherein R₁ is C₄F₉, and wherein R₂ is H. In some embodiments, the Compound 26 comprises a chemical group R1_Extended, wherein R1_Extended is C₆H₅F₉. In some embodiments, the Compound 26 comprises two chemical groups R1_Extended, wherein R1_Extended is C₆H₅F₉. In some embodiments, the Compound 26 comprises two chemical groups R1_Extended, wherein R1_Extended is C₆H₅F₉, and additionally comprises the group R₂, wherein R₂ is H. In some embodiments, Compound 26 comprises Formula VIII_B:

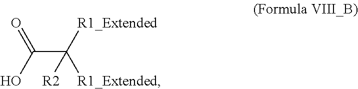

wherein the formula is C(R1_Extended)₂(R2)COOH, wherein R₁_Extended is C₆H₅F₉ and wherein R₂ is H. In some embodiments, Compound 26 comprises the chemical formula 2,2-(R1_Extended)-2-acetic acid, wherein R1_Extended is the chemical group 1,1,1,2,2,3,3,4,4-nonafluorohexane. In some embodiments, Compound 26 comprises 2,2-(1,1,1,2,2,3,3,4,4-nonafluorohexane)-2-acetic acid.

In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 26. In some embodiments, the fluorinated solvent is perfluorohexane and the perfluorinated dispersant compound comprises Compound 26. In some embodiments, the fluorinated solvent is perfluorooctane and the perfluorinated dispersant compound comprises Compound 26. In some embodiments, the fluorinated solvent is perfluoropentane and the perfluorinated dispersant compound comprises Compound 26. In some embodiments, the fluorinated solvent is perfluoroheptane and the perfluorinated dispersant compound comprises Compound 26. In some embodiments, the fluorinated solvent is perfluoromethylcyclohexane and the perfluorinated dispersant compound comprises Compound 26. In some embodiments, the fluorinated solvent is perfluoro-1-3-dimethylcyclohexane and the perfluorinated dispersant compound comprises Compound 26. In some embodiments, the fluorinated solvent is perfluorodecalin and the perfluorinated dispersant compound comprises Compound 26.

In some embodiments, the protein of interest is dispersed in perfluorohexane in the presence of Compound 26. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 26, wherein the molar ratio of Compound 26 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 26, wherein the molar ratio of Compound 26 is about 1000:1 with the protein of interest. In some embodiments, the protein of interest phase separates when dispersed in perfluorohexane in the presence of Compound 26, but does not phase separate when dispersed in perfluorohexane in the absence of Compound 26. In some embodiments, the protein of interest phase separates when dispersed in perfluorohexane in the presence of Compound 26, wherein the dispersion efficiency is about 5%, 10%, 15%, 20%, 25%, 30%, 35%, 40%, 45%, 50%, 55%, 60%, 65%, 70%, 75%, 80%, 85%, 90%, 95%, or about 100%. In some embodiments, the dispersion efficiency is between about 5% and about 100%. In some embodiments, the dispersion efficiency is about 100%. In some embodiments, the dispersion efficiency is at least about 80%. In some embodiments, the dispersion efficiency is at least about 50%. In some embodiments, the dispersion efficiency is at least about 25%. In some embodiments, the dispersion efficiency is at least about 15%.

In some embodiments, the dispersant compound comprises Compound 27. In some embodiments, the Compound 27 comprises a chemical group R1, wherein R1 is C₄F₉. In some embodiments, the Compound 27 comprises two chemical groups R1, wherein R1 is C₄F₉. In some embodiments, the Compound 27 comprises the group R₂, wherein R₂ is CN. In some embodiments, the Compound 27 comprises two chemical groups R1, wherein R1 is C₄F₉, and additionally comprises the group R₂, wherein R₂ is COOH. In some embodiments, Compound 27 comprises Formula VIII:

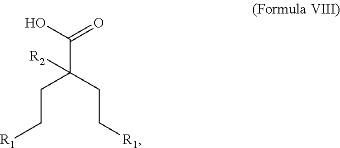

wherein the formula is C₅H₈(R1)₂(R2)COOH, wherein R₁ is C₄F₉, and wherein R₂ is COOH. In some embodiments, the Compound 27 comprises a chemical group R1_Extended, wherein R1_Extended is C₆H₅F₉. In some embodiments, the Compound 27 comprises two chemical groups R1_Extended, wherein R1_Extended is C₆H₅F₉. In some embodiments, the Compound 27 comprises two chemical groups R1_Extended, wherein R1_Extended is C₆H₅F₉, and additionally comprises the group R₂, wherein R₂ is COOH. In some embodiments, Compound 27 comprises Formula VIII_B:

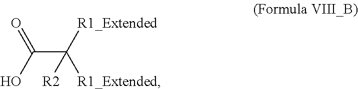

wherein the formula is C(R1_Extended)₂(R2)COOH, wherein R₁ Extended is C₆H₅F₉ and wherein R₂ is COOH. In some embodiments, Compound 27 comprises the chemical formula 2,2-(R1_Extended)-2-diacetic acid, wherein R1_Extended is the chemical group 1,1,1,2,2,3,3,4,4-nonafluorohexane. In some embodiments, Compound 27 comprises 2,2-(1,1,1,2,2,3,3,4,4-nonafluorohexane)-2-diacetic acid.

In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 27. In some embodiments, the fluorinated solvent is perfluorohexane and the perfluorinated dispersant compound comprises Compound 27. In some embodiments, the fluorinated solvent is perfluorooctane and the perfluorinated dispersant compound comprises Compound 27. In some embodiments, the fluorinated solvent is perfluoropentane and the perfluorinated dispersant compound comprises Compound 27. In some embodiments, the fluorinated solvent is perfluoroheptane and the perfluorinated dispersant compound comprises Compound 27. In some embodiments, the fluorinated solvent is perfluoromethylcyclohexane and the perfluorinated dispersant compound comprises Compound 27. In some embodiments, the fluorinated solvent is perfluoro-1-3-dimethylcyclohexane and the perfluorinated dispersant compound comprises Compound 27. In some embodiments, the fluorinated solvent is perfluorodecalin and the perfluorinated dispersant compound comprises Compound 27.

In some embodiments, the protein of interest is dispersed in perfluorohexane in the presence of Compound 27. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 27, wherein the molar ratio of Compound 27 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 27, wherein the molar ratio of Compound 27 is about 1000:1 with the protein of interest. In some embodiments, the protein of interest phase separates when dispersed in perfluorohexane in the presence of Compound 27, but does not phase separate when dispersed in perfluorohexane in the absence of Compound 27. In some embodiments, the protein of interest phase separates when dispersed in perfluorohexane in the presence of Compound 27, wherein the dispersion efficiency is about 5%, 10%, 15%, 20%, 25%, 30%, 35%, 40%, 45%, 50%, 55%, 60%, 65%, 70%, 75%, 80%, 85%, 90%, 95%, or about 100%. In some embodiments, the dispersion efficiency is between about 5% and about 100%. In some embodiments, the dispersion efficiency is about 100%. In some embodiments, the dispersion efficiency is at least about 80%. In some embodiments, the dispersion efficiency is at least about 50%. In some embodiments, the dispersion efficiency is at least about 25%. In some embodiments, the dispersion efficiency is at least about 15%.

In some embodiments, the dispersant compound comprises Compound 28. In some embodiments, the Compound 28 comprises a chemical group R1, wherein R1 is C₆F₁₃. In some embodiments, the Compound 28 comprises two chemical groups R1, wherein R1 is C₆F₁₃. In some embodiments, the Compound 28 comprises the group R₂, wherein R₂ is CN. In some embodiments, the Compound 28 comprises two chemical groups R1, wherein R1 is C₆F₁₃, and additionally comprises the group R₂, wherein R₂ is COOH. In some embodiments, Compound 28 comprises Formula VIII:

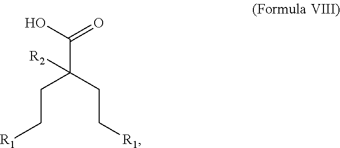

wherein the formula is C₅H₈(R1)₂(R2)COOH, wherein R₁ is C₆F₁₃, and wherein R₂ is COOH. In some embodiments, the Compound 28 comprises a chemical group R1_Extended, wherein R1_Extended is C₈H₅F₁₃. In some embodiments, the Compound 28 comprises two chemical groups R1_Extended, wherein R1_Extended is C₈H₅F₁₃. In some embodiments, the Compound 28 comprises two chemical groups R1_Extended, wherein R1_Extended is C₈H₅F₁₃, and additionally comprises the group R₂, wherein R₂ is COOH. In some embodiments, Compound 28 comprises Formula VIII_B:

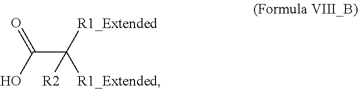

wherein the formula is C(R1_Extended)₂(R2)COOH, wherein R₁_Extended is C₈H₅F₁₃ and wherein R₂ is COOH. In some embodiments, Compound 28 comprises the chemical formula 2,2-(R1_Extended)-2-diacetic acid, wherein R1_Extended is the chemical group 1,1,1,2,2,3,3,4,4,5,5,6,6-tridecafluorooctane. In some embodiments, Compound 28 comprises 2,2-(1,1,1,2,2,3,3,4,4,5,5,6,6-tridecafluorooctane)-2-diacetic acid.

In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 28. In some embodiments, the fluorinated solvent is perfluorohexane and the perfluorinated dispersant compound comprises Compound 28. In some embodiments, the fluorinated solvent is perfluorooctane and the perfluorinated dispersant compound comprises Compound 28. In some embodiments, the fluorinated solvent is perfluoropentane and the perfluorinated dispersant compound comprises Compound 28. In some embodiments, the fluorinated solvent is perfluoroheptane and the perfluorinated dispersant compound comprises Compound 28. In some embodiments, the fluorinated solvent is perfluoromethylcyclohexane and the perfluorinated dispersant compound comprises Compound 28. In some embodiments, the fluorinated solvent is perfluoro-1-3-dimethylcyclohexane and the perfluorinated dispersant compound comprises Compound 28. In some embodiments, the fluorinated solvent is perfluorodecalin and the perfluorinated dispersant compound comprises Compound 28.

In some embodiments, the protein of interest is dispersed in perfluorohexane in the presence of Compound 28. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 28, wherein the molar ratio of Compound 28 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 28, wherein the molar ratio of Compound 28 is about 1000:1 with the protein of interest. In some embodiments, the protein of interest phase separates when dispersed in perfluorohexane in the presence of Compound 28, but does not phase separate when dispersed in perfluorohexane in the absence of Compound 28. In some embodiments, the protein of interest phase separates when dispersed in perfluorohexane in the presence of Compound 28, wherein the dispersion efficiency is about 5%, 10%, 15%, 20%, 25%, 30%, 35%, 40%, 45%, 50%, 55%, 60%, 65%, 70%, 75%, 80%, 85%, 90%, 95%, or about 100%. In some embodiments, the dispersion efficiency is between about 5% and about 100%. In some embodiments, the dispersion efficiency is about 100%. In some embodiments, the dispersion efficiency is at least about 80%. In some embodiments, the dispersion efficiency is at least about 50%. In some embodiments, the dispersion efficiency is at least about 25%. In some embodiments, the dispersion efficiency is at least about 15%.

In some embodiments, the dispersant compound comprises Compound 29. In some embodiments, the Compound 29 comprises a chemical group R1, wherein R1 is C₈F₁₇. In some embodiments, the Compound 29 comprises two chemical groups R1, wherein R1 is C₈F₁₇. In some embodiments, the Compound 29 comprises the group R2, wherein R₂ is CN. In some embodiments, the Compound 29 comprises two chemical groups R1, wherein R1 is C₈F₁₇, and additionally comprises the group R₂, wherein R₂ is COOH. In some embodiments, Compound 29 comprises Formula VIII:

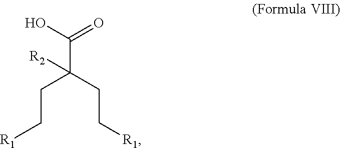

wherein the formula is C₅H₈(R1)₂(R2)COOH, wherein R₁ is C₈F₁₇, and wherein R₂ is COOH. In some embodiments, the Compound 29 comprises a chemical group R1_Extended, wherein R1_Extended is C₁₀H₅F₁₇. In some embodiments, the Compound 29 comprises two chemical groups R1 Extended, wherein R1_Extended is C₁₀H₅F₁₇. In some embodiments, the Compound 29 comprises two chemical groups R1_Extended, wherein R1_Extended is C₁₀H₅F₁₇, and additionally comprises the group R₂, wherein R₂ is COOH. In some embodiments, Compound 29 comprises Formula VIII_B:

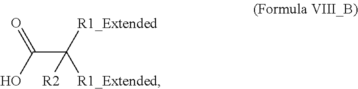

wherein the formula is C(R1_Extended)₂(R2)COOH, wherein R₁_Extended is C₁₀H₅F₁₇ and wherein R₂ is COOH. In some embodiments, Compound 29 comprises the chemical formula 2,2-(R1_Extended)-2-diacetic acid, wherein R1_Extended is the chemical group 1,1,1,2,2,3,3,4,4,5,5,6,6,7,7,8,8-heptadecafluorodecane. In some embodiments, Compound 29 comprises 2,2-(1,1,1,2,2,3,3,4,4,5,5,6,6,7,7,8,8-heptadecafluorodecane)-2-diacetic acid.

In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Compound 29. In some embodiments, the fluorinated solvent is perfluorohexane and the perfluorinated dispersant compound comprises Compound 29. In some embodiments, the fluorinated solvent is perfluorooctane and the perfluorinated dispersant compound comprises Compound 29. In some embodiments, the fluorinated solvent is perfluoropentane and the perfluorinated dispersant compound comprises Compound 29. In some embodiments, the fluorinated solvent is perfluoroheptane and the perfluorinated dispersant compound comprises Compound 29. In some embodiments, the fluorinated solvent is perfluoromethylcyclohexane and the perfluorinated dispersant compound comprises Compound 29. In some embodiments, the fluorinated solvent is perfluoro-1-3-dimethylcyclohexane and the perfluorinated dispersant compound comprises Compound 29. In some embodiments, the fluorinated solvent is perfluorodecalin and the perfluorinated dispersant compound comprises Compound 29.

In some embodiments, the protein of interest is dispersed in perfluorohexane in the presence of Compound 29. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 29, wherein the molar ratio of Compound 29 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 29, wherein the molar ratio of Compound 29 is about 1000:1 with the protein of interest. In some embodiments, the protein of interest phase separates when dispersed in perfluorohexane in the presence of Compound 29, but does not phase separate when dispersed in perfluorohexane in the absence of Compound 29. In some embodiments, the protein of interest phase separates when dispersed in perfluorohexane in the presence of Compound 29, wherein the dispersion efficiency is about 5%, 10%, 15%, 20%, 25%, 30%, 35%, 40%, 45%, 50%, 55%, 60%, 65%, 70%, 75%, 80%, 85%, 90%, 95%, or about 100%. In some embodiments, the dispersion efficiency is between about 5% and about 100%. In some embodiments, the dispersion efficiency is about 100%. In some embodiments, the dispersion efficiency is at least about 80%. In some embodiments, the dispersion efficiency is at least about 50%. In some embodiments, the dispersion efficiency is at least about 25%. In some embodiments, the dispersion efficiency is at least about 15%.

In some embodiments, a composition is provided, the composition comprising a fluorinated solvent, a perfluorinated dispersant compound, and a protein of interest, wherein: the perfluorinated dispersant compound interacts with the protein of interest; the protein of interest is dissolved in the fluorinated solvent; the fluorinated solvent is chosen from perfluoropentane, perfluorohexane, perfluoroheptane, perfluorooctane, perfluoromethylcyclohexane, perfluoro-1,3-dimethylcyclohexane, or perfluorodecalin; and the perfluorinated dispersant comprises Formula IX:

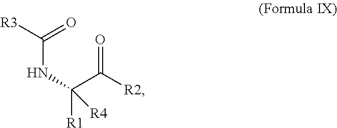

wherein R₁ is CN, H, or COOH, wherein R₂ is the lysosomal targeting group C₇H₁₅N₂O, wherein R₃ is C₄F₉, C₆F₁₃, C₈F₁₇, and wherein R₄ is CN, H, or COOH. In some embodiments, the dispersant compound comprises Formula IX, wherein R₁ is COOH, wherein R₂ is the lysosomal targeting group C₇H₁₅N₂O, wherein R₃ is C₈F₁₇, and wherein R₄ is H. In some embodiments,
the dispersant compound comprises Compound 30:

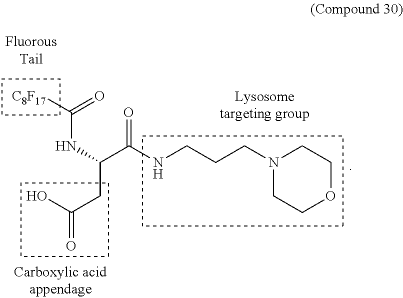

In some embodiments, a composition is provided, the composition comprising a fluorinated solvent, a perfluorinated dispersant compound, and a protein of interest, wherein: the perfluorinated dispersant compound interacts with the protein of interest; the protein of interest is dissolved in the fluorinated solvent; the fluorinated solvent is chosen from perfluoropentane, perfluorohexane, perfluoroheptane, perfluorooctane, perfluoromethylcyclohexane, perfluoro-1,3-dimethylcyclohexane, or perfluorodecalin; and the perfluorinated dispersant compound comprises Formula IX, wherein the compound is Compound 30 comprising the chemical formula that can be derived from the SMILES ID from Table 6.

In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Formula IX. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Formula IX, wherein the compound is Compound 30. In some embodiments, the fluorinated solvent is perfluorohexane and the perfluorinated dispersant compound comprises Formula IX. In some embodiments, the fluorinated solvent is perfluorooctane and the perfluorinated dispersant compound comprises Formula IX. In some embodiments, the fluorinated solvent is perfluoropentane and the perfluorinated dispersant compound comprises Formula IX. In some embodiments, the fluorinated solvent is perfluoroheptane and the perfluorinated dispersant compound comprises Formula IX. In some embodiments, the fluorinated solvent is perfluoromethylcyclohexane and the perfluorinated dispersant compound comprises Formula IX. In some embodiments, the fluorinated solvent is perfluoro-1-3-dimethylcyclohexane and the perfluorinated dispersant compound comprises Formula IX. In some embodiments, the fluorinated solvent is perfluorodecalin and the perfluorinated dispersant compound comprises Formula IX.

In some embodiments, a composition is provided, the composition comprising a fluorinated solvent, a perfluorinated dispersant compound, and a protein of interest, wherein: the perfluorinated dispersant compound interacts with the protein of interest; the protein of interest is dissolved in the fluorinated solvent; the fluorinated solvent is chosen from perfluoropentane, perfluorohexane, perfluoroheptane, perfluorooctane, perfluoromethylcyclohexane, perfluoro-1,3-dimethylcyclohexane, or perfluorodecalin; and the perfluorinated dispersant comprises Formula X:

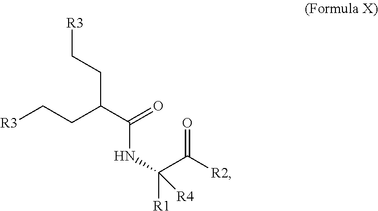

wherein R₁ is CN, H, or COOH, wherein R₂ is the lysosomal targeting group C₇H₁₅N₂O, wherein R₃ is C₄F₉, C₆F₁₃, C₈F₁₇, and wherein R₄ is CN, H, or COOH. In some embodiments, the dispersant compound comprises Formula X, wherein R₁ is COOH, wherein R₂ is the lysosomal targeting group C₇H₁₅N₂O, wherein R₃ is C₆F₁₃, and wherein R₄ is H. In some embodiments,
the dispersant compound comprises Compound 31:

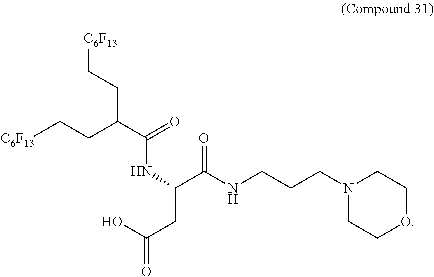

In some embodiments, a composition is provided, the composition comprising a fluorinated solvent, a perfluorinated dispersant compound, and a protein of interest, wherein: the perfluorinated dispersant compound interacts with the protein of interest; the protein of interest is dissolved in the fluorinated solvent; the fluorinated solvent is chosen from perfluoropentane, perfluorohexane, perfluoroheptane, perfluorooctane, perfluoromethylcyclohexane, perfluoro-1,3-dimethylcyclohexane, or perfluorodecalin; and the perfluorinated dispersant compound comprises Formula X, wherein the compound is Compound 31 comprising the chemical formula that can be derived from the SMILES ID from Table 6.

In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Formula X. In some embodiments, the fluorinated solvent is chosen from the group that includes perfluoropentane, perfluorohexane, perfluoroheptane, perfluorooctane, perfluoromethylcyclohexane, perfluoro-1-3-dimethylcyclohexane, or perfluorodecalin, and the perfluorinated dispersant compound comprises Formula X, wherein the compound is Compound 31. In some embodiments, the fluorinated solvent is perfluorohexane and the perfluorinated dispersant compound comprises Formula X. In some embodiments, the fluorinated solvent is perfluorooctane and the perfluorinated dispersant compound comprises Formula X. In some embodiments, the fluorinated solvent is perfluoropentane and the perfluorinated dispersant compound comprises Formula X. In some embodiments, the fluorinated solvent is perfluoroheptane and the perfluorinated dispersant compound comprises Formula X. In some embodiments, the fluorinated solvent is perfluoromethylcyclohexane and the perfluorinated dispersant compound comprises Formula X. In some embodiments, the fluorinated solvent is perfluoro-1-3-dimethylcyclohexane and the perfluorinated dispersant compound comprises Formula X. In some embodiments, the fluorinated solvent is perfluorodecalin and the perfluorinated dispersant compound comprises Formula X.

In some embodiments, the protein of interest is dispersed in perfluorohexane in the presence of a compound comprising Formula IX. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of a compound comprising Formula IX, wherein the molar ratio of a compound comprising Formula IX is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of a compound comprising Formula IX, wherein the molar ratio of a compound comprising Formula IX is about 1000:1 with the protein of interest. In some embodiments, the protein of interest phase separates when dispersed in perfluorohexane in the presence of a compound comprising Formula IX, but does not phase separate when dispersed in perfluorohexane in the absence of a compound comprising Formula IX. In some embodiments, the protein of interest phase separates when dispersed in perfluorohexane in the presence of a compound comprising Formula IX, wherein the dispersion efficiency is about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or about 100%. In some embodiments, the dispersion efficiency is between about 5% and about 100%. In some embodiments, the dispersion efficiency is at least about 10%.

In some embodiments, the protein of interest is dispersed in perfluorohexane in the presence of a compound comprising Formula X. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of a compound comprising Formula X, wherein the molar ratio of a compound comprising Formula X is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of a compound comprising Formula X, wherein the molar ratio of a compound comprising Formula X is about 1000:1 with the protein of interest. In some embodiments, the protein of interest phase separates when dispersed in perfluorohexane in the presence of a compound comprising Formula X, but does not phase separate when dispersed in perfluorohexane in the absence of a compound comprising Formula X. In some embodiments, the protein of interest phase separates when dispersed in perfluorohexane in the presence of a compound comprising Formula X, wherein the dispersion efficiency is about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or about 100%. In some embodiments, the dispersion efficiency is between about 5% and about 100%. In some embodiments, the dispersion efficiency is at least about 10%.

In some embodiments, the protein of interest is dispersed in perfluorohexane in the presence of Compound 30. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 30, wherein the molar ratio of Compound 30 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 30, wherein the molar ratio of Compound 30 is about 1000:1 with the protein of interest. In some embodiments, the protein of interest phase separates when dispersed in perfluorohexane in the presence of Compound 30, but does not phase separate when dispersed in perfluorohexane in the absence of Compound 30. In some embodiments, the protein of interest phase separates when dispersed in perfluorohexane in the presence of Compound 30, wherein the dispersion efficiency is about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or about 100%. In some embodiments, the dispersion efficiency is between about 5% and about 100%. In some embodiments, the dispersion efficiency is at least about 10%.

In some embodiments, the protein of interest is dispersed in perfluorohexane in the presence of Compound 31. In some embodiments, the protein of interest can include, but is not limited to, human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the protein of interest has a molecular weight of at least 5 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, or 100 kDa. In some embodiments, the protein of interest has a molecular weight of about 27 kDa, 64.5 kDa, 66.5 kDa, 76 kDa, or 81 kDa. In some embodiments, the protein of interest has a molecular weight of at least 100 kDa, 110 kDa, 120 kDa, 130 kDa, 140 kDa, 150 kDa, 160 kDa, 170 kDa, 180 kDa, 190 kDa, or 200 kDa. In some embodiments, the protein of interest has a molecular weight of about 150 kDa. In some embodiments, the protein of interest has a molecular weight of at least 200 kDa, 250 kDa, 300 kDa, 350 kDa, 400 kDa, 450 kDa, 500 kDa, 550 kDa, or 600 kDa. In some embodiments, the protein of interest has a molecular weight of about 464 kDa, 483 kDa, or 520 kDa. In some embodiments, the protein of interest is dispersed in perfluorohexane in the presence of Compound 31, wherein the molar ratio of Compound 31 is about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the protein of interest is dispersed in perfluorohexane in the presence of Compound 31, wherein the molar ratio of Compound 31 is about 1000:1 with the protein of interest. In some embodiments, the protein of interest phase separates when dispersed in perfluorohexane in the presence of Compound 31, but does not phase separate when dispersed in perfluorohexane in the absence of Compound 31. In some embodiments, the protein of interest phase separates when dispersed in perfluorohexane in the presence of Compound 31, wherein the dispersion efficiency is about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or about 100%. In some embodiments, the dispersion efficiency is between about 5% and about 100%. In some embodiments, the dispersion efficiency is at least about 10%.

In some embodiments, a composition is provided, the composition comprising a fluorinated solvent, a perfluorinated dispersant compound, and a protein of interest, wherein: the perfluorinated dispersant compound interacts with the protein of interest; the protein of interest is dissolved in the fluorinated solvent; the fluorinated solvent is chosen from perfluoropentane, perfluorohexane, perfluoroheptane, perfluorooctane, perfluoromethylcyclohexane, perfluoro-1,3-dimethylcyclohexane, or perfluorodecalin; and the perfluorinated dispersant compound is chosen from a compound that comprises: Formula I, Formula II, Formula III, Formula IV, Formula V, Formula VI, Formula VII, Formula VIII, Formula IX, or Formula X, or any combination thereof. In some embodiments, a composition is provided, the composition comprising a fluorinated solvent, a perfluorinated dispersant compound, and a protein of interest, wherein: the perfluorinated dispersant compound interacts with the protein of interest; the protein of interest is dissolved in the fluorinated solvent; the fluorinated solvent is chosen from perfluoropentane, perfluorohexane, perfluoroheptane, perfluorooctane, perfluoromethylcyclohexane, perfluoro-1,3-dimethylcyclohexane, or perfluorodecalin; and the perfluorinated dispersant compound is chosen from: perfluorooctane (Compound 1), perfluorotetradecane (Compound 2), trifluoroacetic acid (Compound 3), perfluoropropionic acid (Compound 4), perfluoropentanoic acid (Compound 5), perfluorooctanoic acid (Compound 6), perfluoronanonic acid (Compound 7), perfluorodecanoic acid (Compound 8), perfluoroundecanoic acid (Compound 9), perfluorododecanoic acid (Compound 10), perfluorotetradecanoic acid (Compound 11), perfluorooctadecanoic acid (Compound 12), perfluorocyclohexanecarboxylic acid (Compound 13), (2S) 2-FMOC-amino-3-pentafluorophenyl propanoic acid (Compound 14), pentafluorophenol (Compound 15), pentafluorobenzaldehyde (Compound 16), perfluorooctanesulfonic acid (Compound 17), 1H,1H-heptafluorobutylamine (Compound 18), perfluorooctylamine (Compound 19), and 1H,1H-perfluorononylamine (Compound 20). In some embodiments, the dispersant compound is perfluorinated. In some embodiments, the dispersant compound is a perfluorinated compound chosen from the group that includes: perfluorooctanoic acid (Compound 6), perfluoronanonic acid (Compound 7), perfluorodecanoic acid (Compound 8) perfluoroundecanoic acid (Compound 9), perfluorododecanoic acid (Compound 10), 2,2-(1,1,1,2,2,3,3,4,4,5,5,6,6-tridecafluorooctane)-2-cyanoacetic acid (Compound 21), 2,2-(1,1,1,2,2,3,3,4,4,5,5,6,6-tridecafluorooctane)-2-acetic acid (Compound 22), 2,2-(1,1,1,2,2,3,3,4,4,5,5,6,6,7,7,8,8-heptadecafluorodecane)-2-cyanoacetic acid (Compound 23), 2,2-(1,1,1,2,2,3,3,4,4,5,5,6,6,7,7,8,8-heptadecafluorodecane)-acetic acid (Compound 24), 2,2-(1,1,1,2,2,3,3,4,4-nonafluorohexane)-2-cyanoacetic acid (Compound 25), 2,2-(1,1,1,2,2,3,3,4,4-nonafluorohexane)-2-acetic acid (Compound 26), 2,2-(1,1,1,2,2,3,3,4,4-nonafluorohexane)-2-diacetic acid (Compound 27), 2,2-(1,1,1,2,2,3,3,4,4,5,5,6,6-tridecafluorooctane)-2-diacetic acid (Compound 28), 2,2-(1,1,1,2,2,3,3,4,4,5,5,6,6,7,7,8,8-heptadecafluorodecane)-2-diacetic acid (Compound 29), Compound 30, or Compound 31, or any combination thereof, as described herein.

In some embodiments, a composition is provided, wherein the composition comprises a protein of interest dissolved in the fluorinated solvent perfluorohexane, and a dispersant compound, wherein the dispersant compound interacts with the protein of interest and the dispersant compound comprises the formula of Formula: 1, II, IIT, IV, V, VI, VII, VIII, IX, or X. In some embodiments, the dispersant compound is chosen from Compounds: 1, 2, 3, 4, 5, 6, 7, 8, 9, 10, 11, 12, 13, 14, 15, 16, 17, 18, 19, 20, 6, 7, 8, 9, 10, 21, 22, 23, 24, 25, 26, 27, 28, 29, 30, or 31. In some embodiments, a composition is provided, wherein the composition comprises a protein of interest dissolved in the fluorinated solvent perfluorohexane, and a dispersant compound, wherein the dispersant compound interacts with the protein of interest and the dispersant compound is chosen from Compounds: 1, 2, 3, 4, 5, 6, 7, 8, 9, 10, 11, 12, 13, 14, 15, 16, 17, 18, 19, 20, 6, 7, 8, 9, 10, 21, 22, 23, 24, 25, 26, 27, 28, 29, 30, or 31.

In some embodiments, the protein of interest is human hemoglobin (Hb), green fluorescent protein (GFP), bovine serum albumin (BSA), β-Galactosidase (β-Gal), rabbit serum immunoglobulin (IgG), bovine apo-transferrin (TFN), or urease (URE). In some embodiments, the dispersant compound comprises the formula of Formula: I, II, III, IV, V, VI, VII, VIII, IX, or X; and protein of interest is chosen from the group that includes, but is not limited to: human hemoglobin (Hb), green fluorescent protein (GFP), bovine serum albumin (BSA), β-Galactosidase (β-Gal), rabbit serum immunoglobulin (IgG), bovine apo-transferrin (TFN), and urease (URE). In some embodiments, the dispersant compound is chosen from Compounds: 1, 2, 3, 4, 5, 6, 7, 8, 9, 10, 11, 12, 13, 14, 15, 16, 17, 18, 19, 20, 6, 7, 8, 9, 10, 21, 22, 23, 24, 25, 26, 27, 28, 29, 30, or 31; and protein of interest is chosen from the group that includes, but is not limited to: human hemoglobin (Hb), green fluorescent protein (GFP), bovine serum albumin (BSA), β-Galactosidase (β-Gal), rabbit serum immunoglobulin (IgG), bovine apo-transferrin (TFN), and urease (URE).

In some embodiments, the protein of interest is dispersed in into perfluorohexane in the presence of the dispersion compound, wherein the dispersion compound is at a molar ratio of 1000:1 with the protein, and wherein the dispersion compound is chosen from a compound comprising the formula of Formula: I, II, III, IV, V, VI, VII, VIII, IX, or X. In some embodiments, the protein of interest is dispersed in into perfluorohexane in the presence of the dispersion compound, wherein the dispersion compound is at a molar ratio of 1000:1 with the protein, and wherein the dispersion compound is chosen from Compounds: 1, 2, 3,4,5,6,7, 8,9, 10, 11, 12, 13, 14, 15, 16, 17, 18, 19, 20, 6, 7, 8, 9, 10, 21, 22, 23, 24, 25, 26, 27, 28, 29, 30, or 31.

In some embodiments, the dispersant compound interacts with the protein of interest via hydrogen bonding with nitrogen donors present in the protein backbone and amino acid side chains of the protein of interest, wherein the dispersant compound is chosen from a compound comprising the formula of Formula: I, II, III, IV, V, VI, VII, VIII, IX, or X. In some embodiments, the dispersant compound interacts with the protein of interest via hydrogen bonding with nitrogen donors present in the protein backbone and amino acid side chains of the protein of interest, wherein the dispersant compound is chosen from Compounds: 1, 2, 3, 4, 5, 6, 7, 8, 9, 10, 11, 12, 13, 14, 15, 16, 17, 18, 19, 20, 6, 7, 8, 9, 10, 21, 22, 23, 24, 25, 26, 27, 28, 29, 30, or 31. In some embodiments, the dispersant compound interacts with the protein of interest via hydrogen bonding with nitrogen donors present in the protein backbone and amino acid side chains of the protein of interest, wherein the dispersant compound is at a molar ratio of 1000:1 with the protein.

In some embodiments, the protein of interest comprises a non-native secondary structure. In some embodiments, the dispersant compound interacts with the protein of interest via hydrogen bonding with nitrogen donors present in the protein backbone and amino acid side chains of the protein of interest, wherein the protein of interest comprises a non-native secondary structure, and wherein the dispersant compound is chosen from a compound comprising the formula of Formula: I, II, III, IV, V, VI, VII, VIII, IX, or X. In some embodiments, the dispersant compound interacts with the protein of interest via hydrogen bonding with nitrogen donors present in the protein backbone and amino acid side chains of the protein of interest, wherein the protein of interest comprises a non-native secondary structure, and wherein the dispersant compound is chosen from Compounds: 1, 2, 3, 4, 5, 6, 7, 8, 9, 10, 11, 12, 13, 14, 15, 16, 17, 18, 19, 20, 6, 7, 8, 9, 10, 21, 22, 23, 24, 25, 26, 27, 28, 29, 30, or 31. In some embodiments, the dispersant compound interacts with the protein of interest via hydrogen bonding with nitrogen donors present in the protein backbone and amino acid side chains of the protein of interest, wherein the protein of interest comprises a non-native secondary structure, and wherein the dispersant compound is at a molar ratio of about 100:1, 200:1, 300:1, 400:1, 500:1, 600:1, 700:1, 800:1, 900:1, 1000:1, 1250:1, 1500:1, 1750:1, 2000:1, 2500:1, 3000:1, 4000:1, or 5000:1 with the protein of interest. In some embodiments, the dispersant compound interacts with the protein of interest via hydrogen bonding with nitrogen donors present in the protein backbone and amino acid side chains of the protein of interest, wherein the protein of interest comprises a non-native secondary structure, and wherein the dispersant compound is at a molar ratio of about 1000:1 with the protein.

In some embodiments, the protein of interest phase separates when dispersed in perfluorohexane in the presence of the dispersion compound comprising the formula of Formula: I, II, III, IV, V, VI, VII, VIII, IX, or X, wherein the dispersion efficiency is at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95%. In some embodiments, the protein of interest phase separates when dispersed in perfluorohexane in the presence of the dispersion compound comprising the formula of Formula: I, II, III, IV, V, VI, VII, VIII, IX, or X, wherein the dispersion efficiency is at least about 10%, 15%, 20%, 25%, 30%, 35%, 40%, 45%, 50%, 55%, 60%, 65%, 70%, 75%, 80%, 85%, 90%, or 95%. In some embodiments, the protein of interest phase separates when dispersed in perfluorohexane in the presence of the dispersion compound comprising the formula of Formula: I, II, III, IV, V, VI, VII, VIII, IX, or X, wherein the dispersion efficiency is at least about 95%, 96%, 97%, 98%, or 99%. In some embodiments, the protein of interest phase separates when dispersed in perfluorohexane in the presence of the dispersion compound comprising the formula of Formula: I, II, III, IV, V, VI, VII, VIII, IX, or X, wherein the dispersion efficiency is between about 10% to about 100%. In some embodiments, the protein of interest phase separates when dispersed in perfluorohexane in the presence of the dispersion compound comprising the formula of Formula: I, II, III, IV, V, VI, VII, VIII, IX, or X, wherein the dispersion efficiency is about 100%. In some embodiments, the protein of interest phase separates when dispersed in perfluorohexane in the presence of the dispersion compound comprising the formula of Formula: I, II, III, IV, V, VI, VII, VIII, IX, or X, wherein the dispersion efficiency is about at least 80%. In some embodiments, the protein of interest phase separates when dispersed in perfluorohexane in the presence of the dispersion compound comprising the formula of Formula: I, II, III, IV, V, VI, VII, VIII, IX, or X, wherein the dispersion efficiency is at least about 50%. In some embodiments, the protein of interest phase separates when dispersed in perfluorohexane in the presence of the dispersion compound comprising the formula of Formula: I, II, III, IV, V, VI, VII, VIII, IX, or X, wherein the dispersion efficiency is at least about 25%. In some embodiments, the protein of interest phase separates when dispersed in perfluorohexane in the presence of the dispersion compound comprising the formula of Formula: I, II, III, IV, V, VI, VII, VIII, IX, or X, wherein the dispersion efficiency is at least about 10%. In some embodiments, the protein of interest phase separates when dispersed in perfluorohexane in the presence of the dispersion compound comprising the formula of Formula: I, II, III, IV, V, VI, VII, VIII, IX, or X, wherein the dispersion efficiency is at least about 5%.

In some embodiments, the protein of interest phase separates when dispersed in perfluorohexane in the presence of the dispersion Compound 1, 2, 3, 4, 5, 6, 7, 8, 9, 10, 11, 12, 13, 14, 15, 16, 17, 18, 19, 20, 6, 7, 8, 9, 10, 21, 22, 23, 24, 25, 26, 27, 28, 29, 30, or 31, wherein the dispersion efficiency is at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, or 95%. In some embodiments, the protein of interest phase separates when dispersed in perfluorohexane in the presence of the dispersion Compound 1, 2, 3, 4, 5, 6, 7, 8, 9, 10, 11, 12, 13, 14, 15, 16, 17, 18, 19, 20, 6, 7, 8, 9, 10, 21, 22, 23, 24, 25, 26, 27, 28, 29, 30, or 31, wherein the dispersion efficiency is at least about 10%, 15%, 20%, 25%, 30%, 35%, 40%, 45%, 50%, 55%, 60%, 65%, 70%, 75%, 80%, 85%, 90%, or 95%. In some embodiments, the protein of interest phase separates when dispersed in perfluorohexane in the presence of the dispersion Compound 1, 2, 3, 4, 5, 6, 7, 8, 9, 10, 11, 12, 13, 14, 15, 16, 17, 18, 19, 20, 6, 7, 8, 9, 10, 21, 22, 23, 24, 25, 26, 27, 28, 29, 30, or 31, wherein the dispersion efficiency is at least about 95%, 96%, 97%, 98%, or 99%. In some embodiments, the protein of interest phase separates when dispersed in perfluorohexane in the presence of the dispersion Compound 1, 2, 3, 4, 5, 6, 7, 8, 9, 10, 11, 12, 13, 14, 15, 16, 17, 18, 19, 20, 6, 7, 8, 9, 10, 21, 22, 23, 24, 25, 26, 27, 28, 29, 30, or 31, wherein the dispersion efficiency is between about 10% to about 100%. In some embodiments, the protein of interest phase separates when dispersed in perfluorohexane in the presence of the dispersion Compound 1, 2, 3, 4, 5, 6, 7, 8, 9, 10, 11, 12, 13, 14, 15, 16, 17, 18, 19, 20, 6, 7, 8, 9, 10, 21, 22, 23, 24, 25, 26, 27, 28, 29, 30, or 31, wherein the dispersion efficiency is about 100%. In some embodiments, the protein of interest phase separates when dispersed in perfluorohexane in the presence of the dispersion Compound 1, 2, 3, 4, 5, 6, 7, 8, 9, 10,11, 12, 13, 14, 15, 16, 17, 18, 19, 20, 6,7, 8, 9, 10, 21, 22, 23, 24, 25, 26, 27, 28, 29, 30, or 31, wherein the dispersion efficiency is about at least 80%. In some embodiments, the protein of interest phase separates when dispersed in perfluorohexane in the presence of the dispersion Compound 1, 2, 3, 4, 5, 6, 7, 8, 9, 10, 11, 12, 13, 14, 15, 16, 17, 18, 19, 20, 6, 7, 8, 9, 10, 21, 22, 23, 24, 25, 26, 27, 28, 29, 30, or 31, wherein the dispersion efficiency is at least about 50%. In some embodiments, the protein of interest phase separates when dispersed in perfluorohexane in the presence of the dispersion Compound 1, 2, 3, 4, 5, 6, 7, 8, 9, 10, 11, 12, 13, 14, 15, 16, 17, 18, 19, 20, 6, 7, 8, 9, 10, 21, 22, 23, 24, 25, 26, 27, 28, 29, 30, or 31, wherein the dispersion efficiency is at least about 25%. In some embodiments, the protein of interest phase separates when dispersed in perfluorohexane in the presence of the dispersion Compound 1, 2, 3, 4, 5, 6, 7, 8, 9, 10, 11, 12, 13, 14, 15, 16, 17, 18, 19, 20, 6, 7, 8, 9, 10, 21, 22, 23, 24, 25, 26, 27, 28, 29, 30, or 31, wherein the dispersion efficiency is at least about 10%. In some embodiments, the protein of interest phase separates when dispersed in perfluorohexane in the presence of the dispersion Compound 1, 2, 3, 4, 5, 6, 7, 8, 9, 10, 11, 12, 13, 14, 15, 16, 17, 18, 19, 20, 6, 7, 8, 9, 10, 21, 22, 23, 24, 25, 26, 27, 28, 29, 30, or 31, wherein the dispersion efficiency is at least about 5%.

In some embodiments, the protein of interest dissolved in PFH in the presence of PFOA, PFNA, or PFOS wherein the protein of interest is taken into cells. In some embodiments, the cells can include, but are not limited to, human cells, animal cells, plant cells, insect cells, or any combination thereof. In some embodiments, the cells can include, but are not limited to, primary cells, transformed cells, self-renewing cells, any cell type that can be cultured in suspension culture, or any combination thereof. Self-renewing cells can include immortal cell lines, stem cells, or any combination thereof. In some embodiments, the cells comprise immortal cell lines. Immortal cell lines can include, but are not limited to, Chinese Hamster Ovary (CHO) cells, Henrietta Lacks (HeLa) cells, H-9 cells, Jurkat cells, C6/36 cells, High Five cells, Schneider 2 (S2) cells, Spodoptera frugiperda 21 (Sf21) cells, Spodoptera frugiperda 9 (Sf9) cells, SH-SY5Y cells, A549 cells, or PC-3 cells. In some embodiments, the cells comprise stem cells. Stem cells can include, but are not limited to, embryonic stem cells, adult stem cells, or induced pluripotent stem cells. Embryonic stem cells can include, but are not limited to, any embryonic stem cell with qualities of pluripotency. Pluripotency refers to the ability to give rise to cell types representative of all the embryonic tissues and representative of all adult tissues when differentiated. Adult stem cells can include, but are not limited to, hematopoietic stem cells, mesenchymal stem cells, fibroblast stem cells, any tissue-specific stem cell, or any combination thereof. Tissue-specific stem cells can include, but are not limited to, neural stem cells, epithelial stem cells, and skin stem cells. Induced pluripotent stem cells can be any stem cell derived from adult stem cells that has been induced for pluripotency. Induced pluripotent stem cells can include, but are not limited to induced mesenchymal stem cells, induced fibroblast stem cells, induced neural stem cells, induced epithelial stem cells, and induced skin stem cells. In some embodiments, the protein of interest is taken up into cells at an efficiency of at least 50%.

In some embodiments, the protein of interest is labeled with a fluorophore.

In some embodiments, a method of dissolving a protein of interest in a fluorinated solvent is provided, the method comprising: contacting the protein of interest with a dispersant compound, and dissolving the protein of interest and dispersant compound in the fluorinated solvent. In some embodiments, the method comprises any of the compositions described in the above embodiments.

In some embodiments, the method of dissolving a protein of interest in a fluorinated solvent comprises a fluorinated solvent selected from the group consisting of perfluoropentane, perfluorohexane, perfluoroheptane, perfluorooctane, perfluoromethylcyclohexane, perfluoro-1,3-dimethylcyclohexane, and perfluorodecalin. In some embodiments, the fluorinated solvent is perfluorohexane. In some embodiments, the fluorinated solvent is perfluorooctane.

In some embodiments, the method of dissolving a protein of interest in a fluorinated solvent comprises a dispersant compound, wherein the dispersant compound is perfluorinated. In some embodiments, the dispersant compound comprises Formula II:

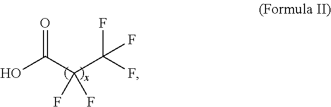

wherein x is 6, 7, 8, 9, 10, 12, or 16. In some embodiments, x is 7 (perfluoronanonic acid). In some embodiments, the dispersant compound comprises Formula III:

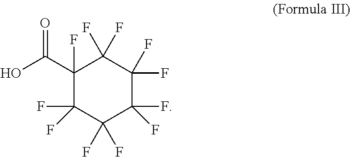 [SMILES: O=C(O)C1(F)C(F)(F)C(F)(F)C(F)(F)C(F)(F)C1(F)F]

In some embodiments, the dispersant compound comprises Formula V:

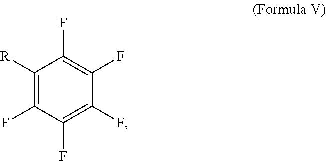

wherein R is OH or COH. In some embodiments, the dispersant compound comprises Formula VI:

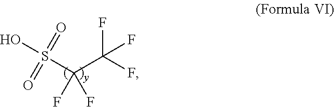

wherein y is 7. In some embodiments,
the dispersant compound comprises Formula VIII:

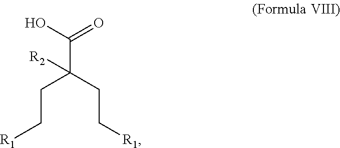

wherein R₁ is C₆F₁₃, C₈F₁₇, or C₄F₉, and wherein R₂ is CN, H, or COOH. In some embodiments, R₁ is C₆F₁₃ and R₂ is H.

In some embodiments, the method of dissolving a protein of interest in a fluorinated solvent comprises a dispersant compound, wherein the dispersant compound interacts with the protein of interest via hydrogen bonding with nitrogen donors present in the protein backbone and amino acid side chains of the protein of interest. In some embodiments, the protein of interest comprises a non-native secondary structure.

In some embodiments, the method of dissolving a protein of interest in a fluorinated solvent comprises a fluorinated solvent, wherein the fluorinated solvent is perfluorohexane and the dispersant compound is perfluoronanonic acid. In some embodiments, the fluorinated solvent is perfluorooctane and the dispersant compound comprises Formula VIII:

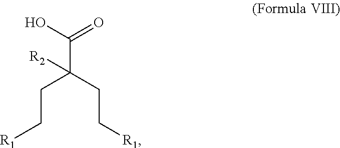

wherein R₁ is C₆F₁₃ and R₂ is H.

### ENUMERATED EMBODIMENTS

The following examples are illustrative, but not limiting, of the compounds, compositions and methods described herein. Other suitable modifications and adaptations known to those skilled in the art are within the scope of the following embodiments.

Embodiment 1. A composition comprising a fluorinated solvent, a dispersant compound, and a protein of interest, wherein:
the dispersant compound interacts with the protein of interest; and
the protein of interest is dissolved in the fluorinated solvent.
   Embodiment 2. The composition of embodiment 1, wherein the fluorinated solvent is selected from the group consisting of perfluoropentane, perfluorohexane, perfluoroheptane, perfluorooctane, perfluoromethylcyclohexane, perfluoro-1,3-dimethylcyclohexane, and perfluorodecalin.
   Embodiment 3. The composition of embodiments 1 or 2, wherein the fluorinated solvent is perfluorohexane.
   Embodiment 4. The composition of embodiments 1 or 2, wherein the fluorinated solvent is perfluorooctane.
   Embodiment 5. The composition of any proceeding embodiment, wherein the dispersant compound is perfluorinated.
   Embodiment 6. The composition of embodiment 5, wherein the dispersant compound comprises Formula II:

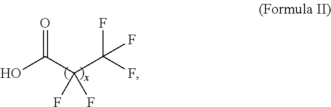

wherein x is 6, 7, 8, 9, 10, 12, or 16.
Embodiment 7. The composition of embodiment 6, wherein x is 7 (perfluoronanonic acid).
Embodiment 8. The composition of embodiment 5, wherein the dispersant compound comprises Formula III:

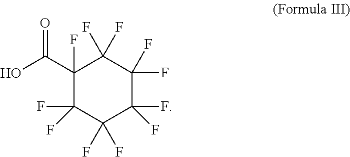 [SMILES: O=C(O)C1(F)C(F)(F)C(F)(F)C(F)(F)C(F)(F)C1(F)F]

Embodiment 9. The composition of embodiment 5, wherein the dispersant compound comprises Formula V:

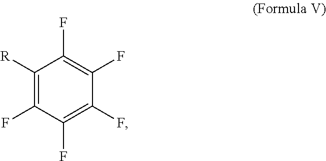

wherein R is OH or COH.
   Embodiment 10. The composition of embodiment 5, wherein the dispersant compound comprises Formula VI:

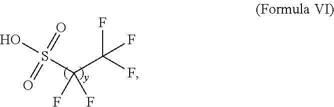

wherein y is 7.
   Embodiment 11. The composition of embodiment 5, wherein the dispersant compound comprises Formula VIII:

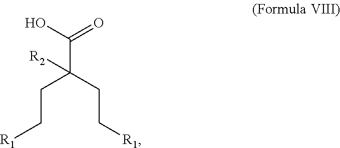

wherein R₁ is C₆F₁₃, C₈F₁₇, or C₄F₉, and
wherein R₂ is CN, H, or COOH.
   Embodiment 12. The composition of embodiment 11, wherein R₁ is C₆F₁₃ and R₂ is H.
   Embodiment 13. The composition of any proceeding embodiment, wherein the dispersant compound interacts with the protein of interest via hydrogen bonding with nitrogen donors present in the protein backbone and amino acid side chains of the protein of interest.
   Embodiment 14. The composition of any proceeding embodiments, wherein the protein of interest comprises a non-native secondary structure.
   Embodiment 15. The composition of embodiment 1, wherein the fluorinated solvent is perfluorohexane and the dispersant compound is perfluoronanonic acid.
   Embodiment 16. The composition of embodiment 1, wherein the fluorinated solvent is perfluorooctane and the dispersant compound comprises Formula VIII:

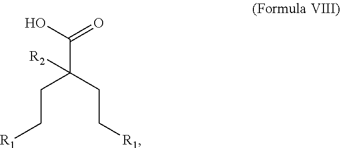

wherein R₁ is C₆F₁₃ and R₂ is H.
   Embodiment 16. A method of dissolving a protein of interest in a fluorinated solvent, the method comprising:
contacting the protein of interest with a dispersant compound, and
dissolving the protein of interest and dispersant compound in the fluorinated solvent.
   Embodiment 17. The method of embodiment 16, wherein the fluorinated solvent is selected from the group consisting of perfluoropentane, perfluorohexane, perfluoroheptane, perfluorooctane, perfluoromethylcyclohexane, perfluoro-1,3-dimethylcyclohexane, and perfluorodecalin.
   Embodiment 18. The method of embodiment 16 or 17, wherein the fluorinated solvent is perfluorohexane.
   Embodiment 19. The method of embodiment 16 or 17, wherein the fluorinated solvent is perfluorooctane.
   Embodiment 20. The method of any one of embodiments 16-19, wherein the dispersant compound is perfluorinated.
   Embodiment 21. The method of embodiment 20, wherein the dispersant compound comprises Formula II:

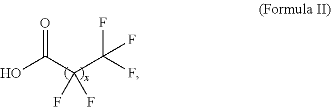

wherein x is 6, 7, 8, 9, 10, 12, or 16.
   Embodiment 22. The method of embodiment 21, wherein x is 7 (perfluoronanonic acid).
   Embodiment 23. The method of embodiment 20, wherein the dispersant compound comprises Formula III:

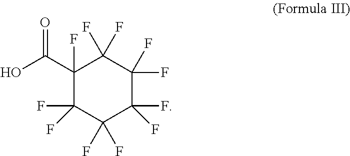 [SMILES: O=C(O)C1(F)C(F)(F)C(F)(F)C(F)(F)C(F)(F)C1(F)F]

Embodiment 24. The method of embodiment 20, wherein the dispersant compound comprises Formula V:

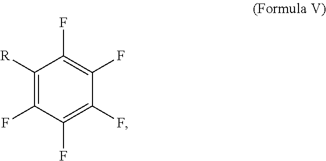

wherein R is OH or COH.
   Embodiment 25. The method of embodiment 20, wherein the dispersant compound comprises Formula VI:

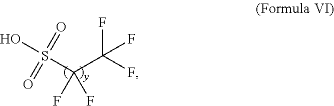

wherein y is 7.
   Embodiment 26. The method of embodiment 20, wherein the dispersant compound comprises Formula VIII:

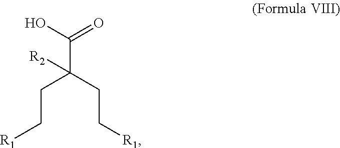

wherein R₁ is C₆F₁₃, C₈F₁₇, or C₄F₉, and
wherein R₂ is CN, H, or COOH.
   Embodiment 27. The method of embodiment 26, wherein R₁ is C₆F₁₃ and R₂ is H.
   Embodiment 28. The method of any one of embodiments 16-27, wherein the dispersant compound interacts with the protein of interest via hydrogen bonding with nitrogen donors present in the protein backbone and amino acid side chains of the protein of interest.
   Embodiment 29. The method of any one of embodiments 16-28, wherein the protein of interest comprises a non-native secondary structure.
   Embodiment 30. The method of embodiment 16, wherein the fluorinated solvent is perfluorohexane and the dispersant compound is perfluoronanonic acid.
   Embodiment 31. The method of embodiment 16, wherein the fluorinated solvent is perfluorooctane and the dispersant compound comprises Formula VIII:

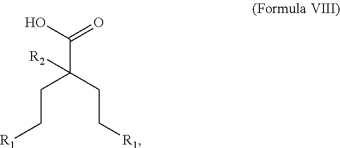

wherein R₁ is C₆F₁₃ and R₂ is H.

### EXAMPLES

The following Examples are presented to illustrate various aspects of the present application but are not intended to limit the scope of the disclosures herein.

### Example 1: Deciphering the Mechanistic Basis for Perfluoroalkyl-Protein Interactions

Although rarely used in nature, fluorine has emerged as an important elemental ingredient in the design of proteins with altered folding, stability, oligomerization propensities and bioactivity. Here, a molecular modification toolkit is described, wherein the ability of privileged perfluorinated amphiphiles to non-covalently decorate proteins is used to alter protein conformational plasticity and potentiate protein dispersion into fluorous phases. A complementary suite of biophysical, in silico and in vitro approaches were used to establish structure-activity relationships defining these phenomena and their impact on protein structural dynamics and intracellular trafficking. Notably, the lead compound, perfluoronanonic acid, was 10⁶ more potent in inducing non-native protein secondary structure compared to the well-known helix inducer trifluoroethanol. Perfluoronanonic acid additionally significantly enhanced the cellular uptake of complexed proteins. These findings may advance the rational design of fluorinated proteins, inform on potential modes of toxicity for perfluoroalkyl substances, and guide the development of fluorine-modified biologics with desirable functional properties for drug discovery and delivery applications.

### Introduction

Perfluoroalkyl substances (PFAS) are organofluorine compounds that possess C—F bonds and other heteroatom functional groups (e.g. —OH, —CO₂H). These chemicals are utilized in several industries, including automotive, household goods, construction, electronics, and biomedicine. As examples in chemical biology, PFAS represent key ingredients in supramolecular biomaterials, for use as functional handles in bio-imaging and -spectroscopy probes, and as chemical tags in metabolomic/proteomic studies. These applications frequently rely on the chemically and biologically inert nature of fluorinated compounds. However, this long-standing paradigm is being revisited as evidence suggests perfluoroalkyl compounds elicit a plurality of bioeffects in cells.

Early evidence of bio-interactions previously emerged when groups reported the ability of perfluorinated amphiphiles to insert into hydrocarbon lipid bilayers and phase-separate into fluorine-rich microdomains. This phenomenon has since been exploited to develop fluorinated nano-carriers and drug delivery technologies that permit the intracellular transport of otherwise membrane-impermeable biologic cargo. However, not all these interactions are constructive. A growing body of evidence demonstrates toxicologic effects of certain PFAS chemicals, of which a select group are now recognized as environmental contaminants. A common observation across these prior studies is that minute changes in PFAS composition and structure can have profound effects on their bioactivity. Yet, thorough structure-activity relationships defining these outcomes are lacking.

Here, it is shown that, in addition to their known ability to interact with cellular lipids, select PFAS chemicals noncovalently adsorb to proteins and allow proteins to partition into fluorous phases. Utilizing complementary biophysical, chemical, and computational assays, the mechanistic basis for PFAS-protein interactions was probed. Fundamental structure-activity relationships defining the association of select PFAS chemicals and proteins are delineated, and their impact on protein conformational dynamics and intracellular trafficking are investigated. In summary, the findings reveal previously unknown interactions of PFAS compounds with a wide variety of protein classes. This work may enable a deeper understanding of the bioeffects of PFAS chemicals and lead to the identification of new fluorochemical-mediated protein formulation methods.

### Results and Discussion

### Screening Perfluoroalkyl-Protein Interaction Affinity

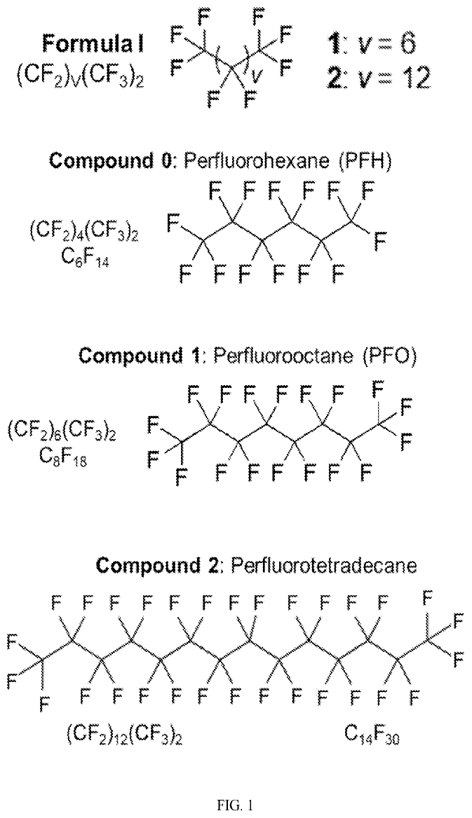

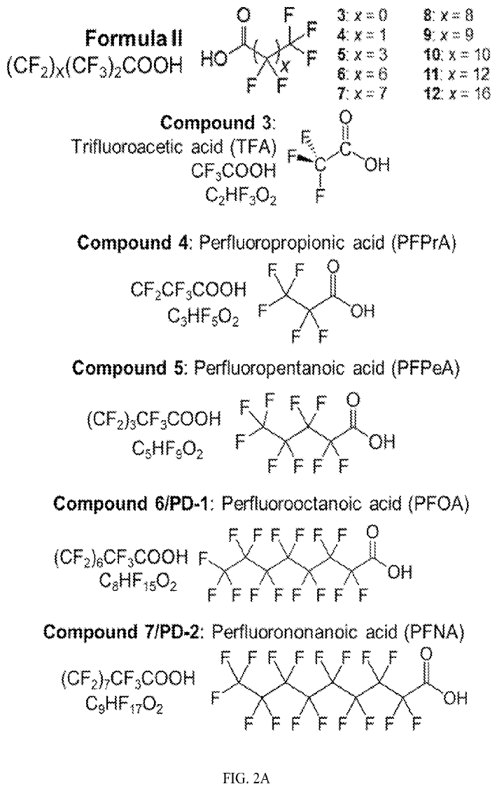

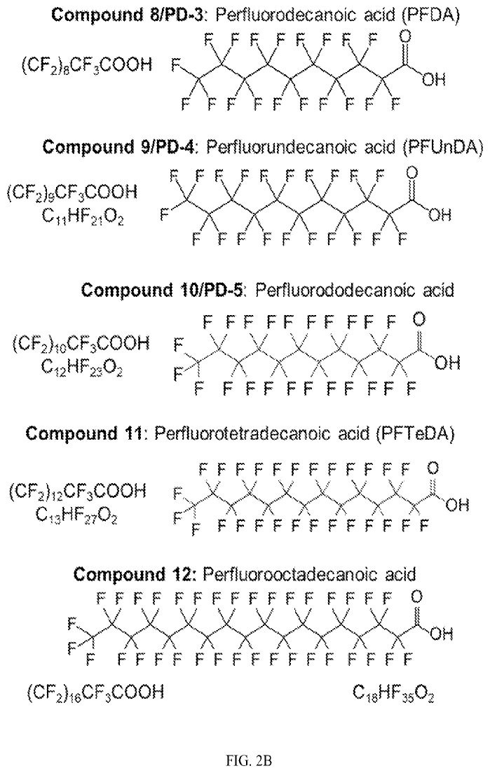

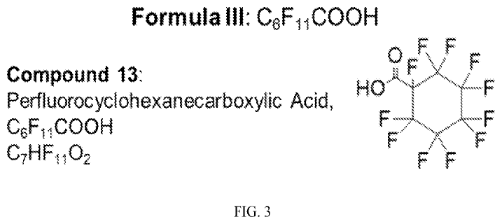

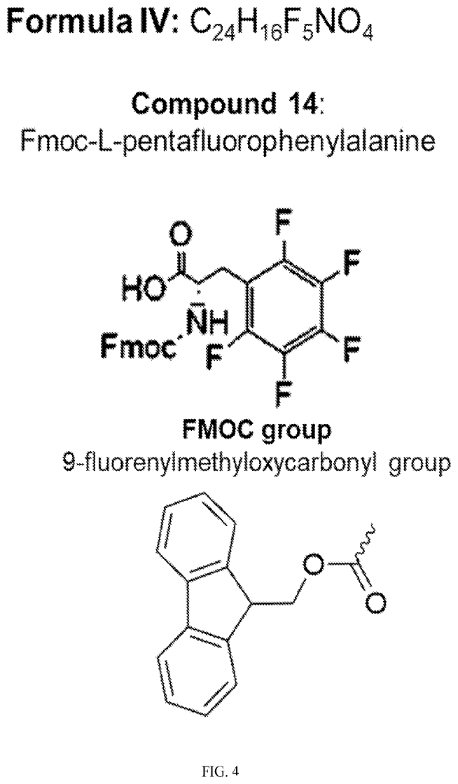

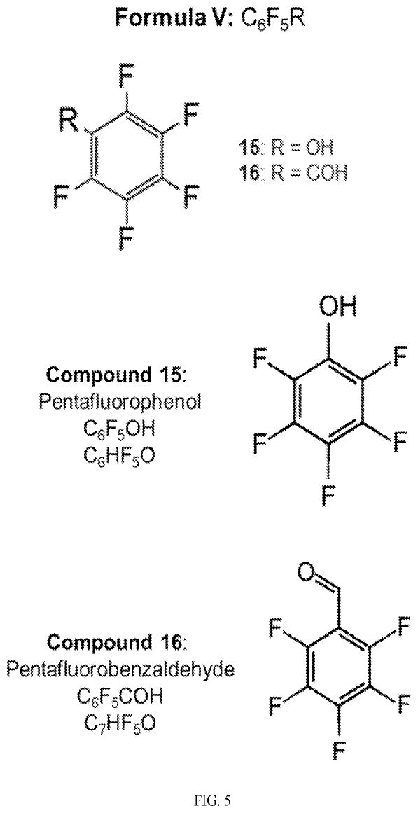

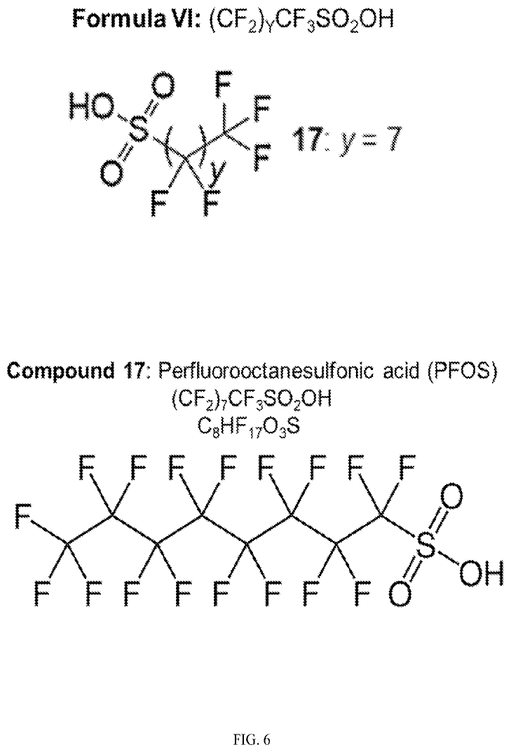

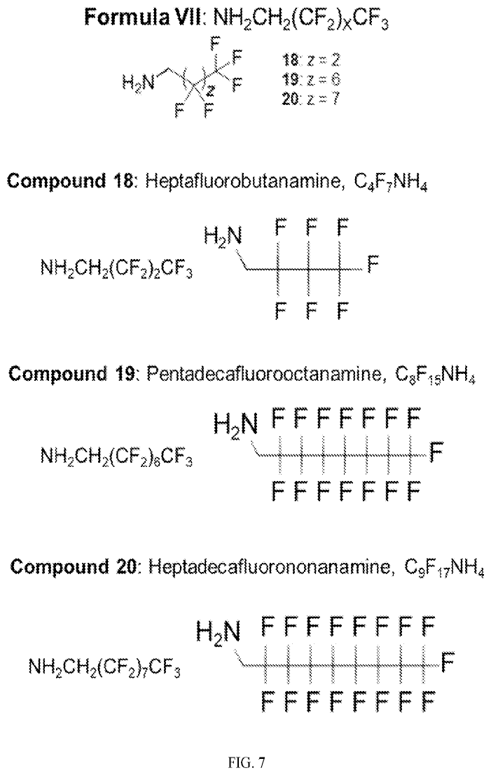

Perfluorinated compounds possess unusual physicochemical properties that result from the juxtaposition of weak intermolecular forces, caused by the low polarizability of fluorines, and the strong intramolecular C—F bond. For PFAS amphiphiles, the combination of polar head group and fluorophilic tail additionally leads to their preferential assembly at water-fluorous interfaces. Based on these unique properties, it was speculated that PFAS compounds may noncovalently associate with proteins to mediate their phase separation into fluorous media. To test this, a screening assay was developed to qualitatively investigate the binding affinity of a library of perfluorinated chemicals with seven model proteins, which include human hemoglobin (Hb), green fluorescent protein (GFP), bovine serum albumin (BSA), (β-Galactosidase (β-Gal), rabbit serum immunoglobulin (IgG), bovine apo-transferrin (TFN), and urease (URE). This group of model proteins captures an array of physicochemical properties ranging in molecular weight, solvent accessible surface area (SASA), isoelectric point (pI), and hydrodynamic radii. A corresponding perfluorinated small molecule test set was selected to contain a plurality of functional groups, linear perfluorocarbon chain lengths, and cyclic or aromatic geometries (FIGS. 1-7). Selection of these compounds was based on their potential to form noncovalent bonds (i.e., hydrogen or ionic) with the solvent exposed protein surfaces. Notably, perfluorooctanoic (PFOA, 6), perfluoronanonic (PFNA, 7), and perfluorooctanesulfonic (PFOS, 17) acids were included. These three linear PFAS chemicals are currently regulated by the US Environmental Protection Agency based on their reproductive, immune, and hepatic toxicity (References: 44-46). In sum, the structural and biochemical diversity represented in the protein and fluorochemical libraries was rationally selected to allow structure-function-performance relationships to be ascertained from binding studies.

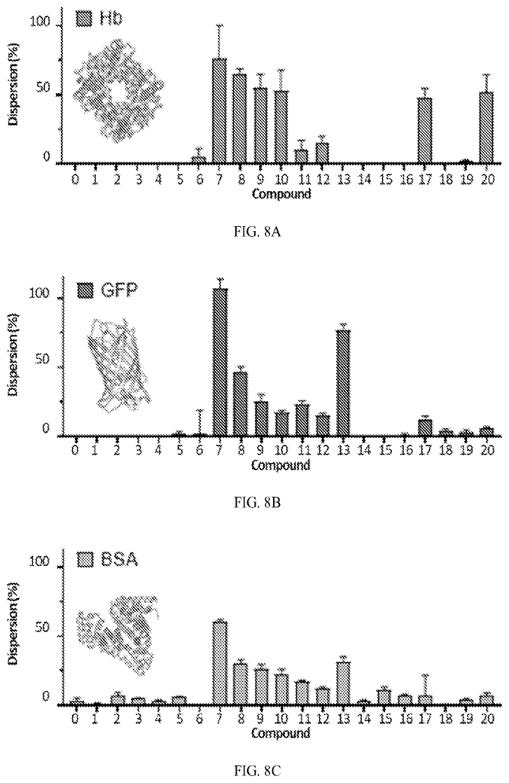

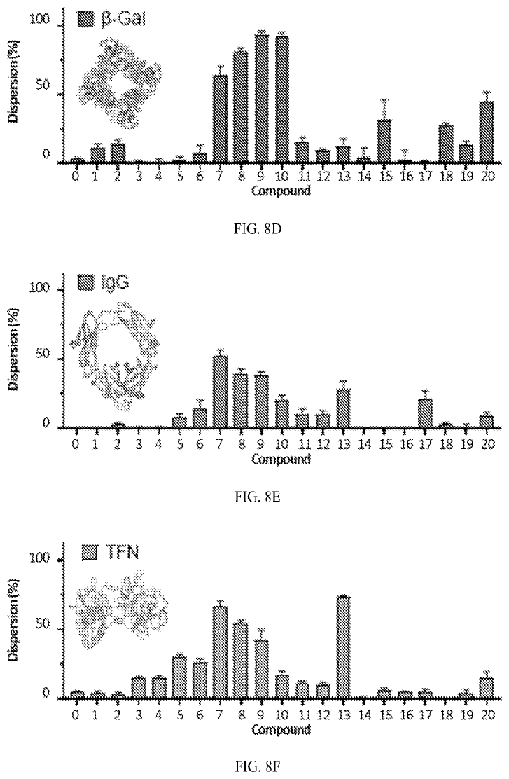

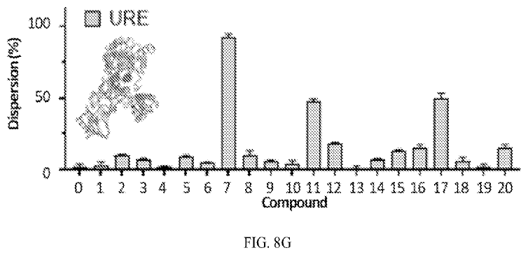

To investigate PFAS-protein binding avidity, an assay was developed to measure the concentration of protein dispersed into perfluorohexane (PFH) in the presence of each fluorochemical compound (1000:1 molar ratio with protein). Protein dispersion into PFH served as a quantitative measure of PFAS-protein avidity, where the amount of dispersed protein was normalized to its initial concentration to evaluate binding interactions. Results (FIG. 8A-8G) showed that, in the absence of a fluorochemical dispersant, all seven proteins were insoluble within PFH (see compound 0). However, compound 7, the ionic perfluoroalkyl PFNA, showed a marked capacity to phase separate all of the tested proteins into the fluorous solvent, with dispersion efficiencies of 53%-100%. Perfluorocyclohexanecarboxylic acid (compound 13), PFOS (compound 17) and 1H,1H-Perfluorononylamine also demonstrated a capacity to disperse many of the protein candidates into PFH; however, their behavior was not as consistent or as potent as PFNA.

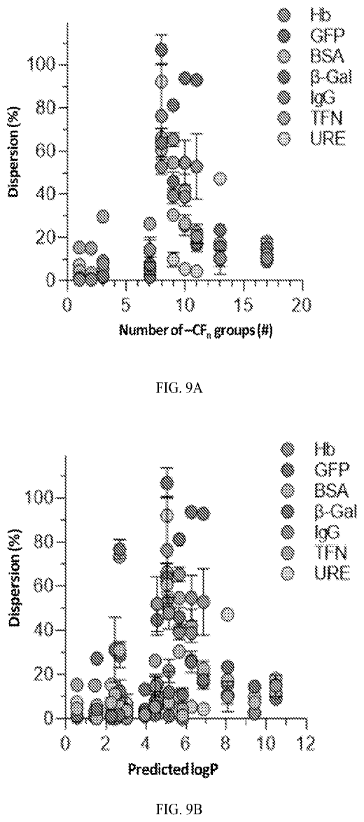

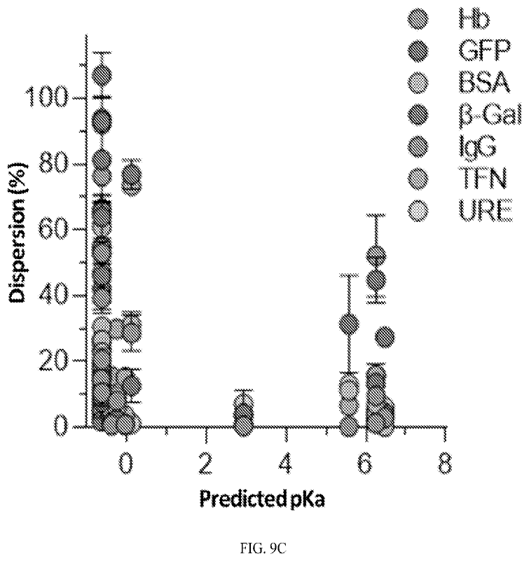

Two interesting conclusions emerged from this data. The first is that carboxylic acids possessed superior binding avidity relative to other heteroatom functional groups represented in the compound library. The second is a distinct optimum of the tail length at eight perfluorocarbons (FIG. 9A). In fact, subtraction or addition of a single —CF₂ group from/to PFNA (e.g., compound 6 and 8, respectively) significantly compromised its association with most proteins. The notable exception was β-Gal, which showed a stepwise increase in dispersion efficiency as the perfluorocarbon tail was increased from —(CF)₇CF₃ (compound 7) to —(CF)₁₀CF₃ (compound 10). A sharp drop off in dispersion efficiency was observed at —(CF)₁₂CF₃ (compound 11). This demonstrates that, although PFNA was optimal for the dispersion of most proteins, high molecular weight biomacromolecules, like β-Gal (MW=464 kDa, the largest in the studied set), required a slightly longer chain length to produce a stable fluorous coating and maximize fluorous dispersion. More broadly, the data suggested that there exists a threshold of fluorine content required to efficiently solubilize PFAS decorated proteins into fluorous media. Increasing the fluorine content beyond this threshold may promote fluorine-fluorine driven association to generate supramolecular assemblies and/or protein flocculates; an assertion that was further supported by the observation of protein precipitates in the low dispersion efficiency trials. These large aggregates were subsequently removed during the centrifugation step of the screening assay to ensure only soluble protein were measured.

In addition to an optimal tail length (—(CF₂)₇CF₃), further analyses revealed an ideal log P of ~5 for fluorochemical protein dispersion (FIG. 9B). Conversely, a correlation between fluoroamphiphile dissociation constant (pKₐ) and dispersion trends was not observed (FIG. 9C). Taken together, this data suggested that four properties were important to PFAS-protein interaction avidity. First, perfluorinated carboxylic acids were found to be privileged among the functional groups tested. Secondly, an amphiphilic character that balanced interaction of the fluorochemical with polar protein surfaces and the fluorous solvent was found to be preferred. Next, it was necessary that the compounds possess an acidic proton, but there was no correlation between PFAS pKa and protein adsorption. Finally, linear perfluorocarbons outperformed cyclic and aromatic species.

### Structure-Function-Performance Relationships of PFNA-Protein Complexation

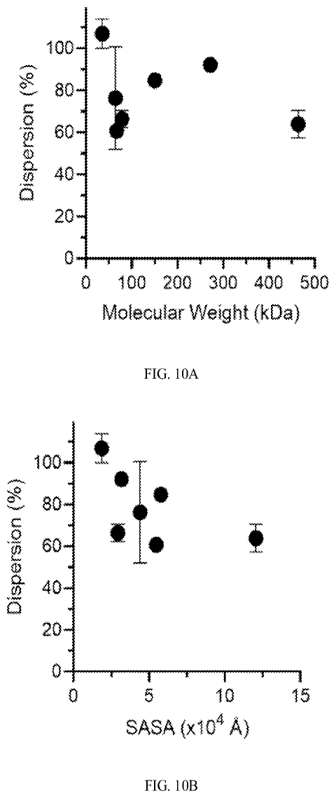

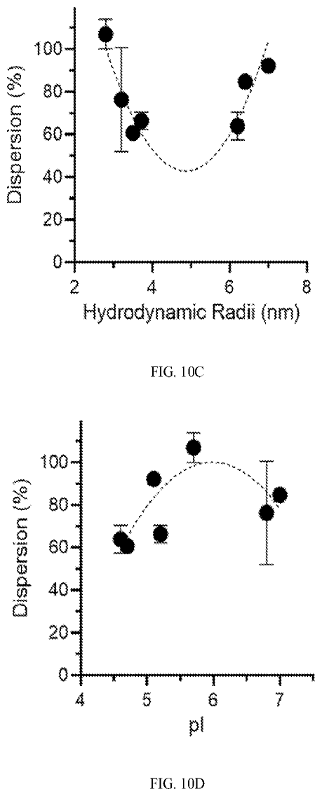

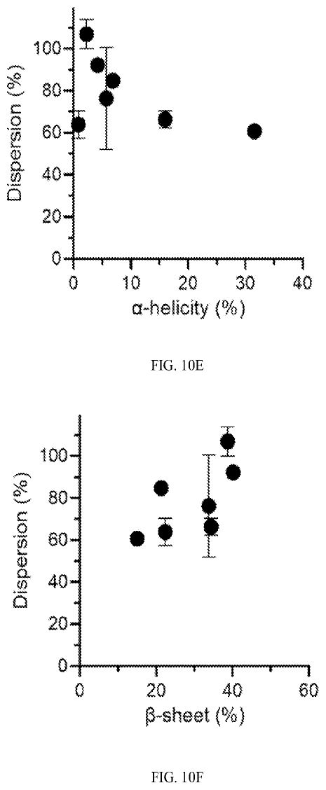

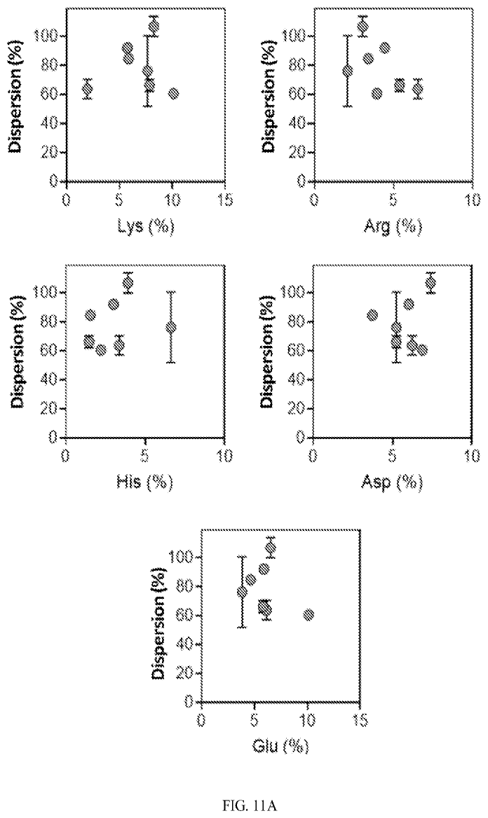

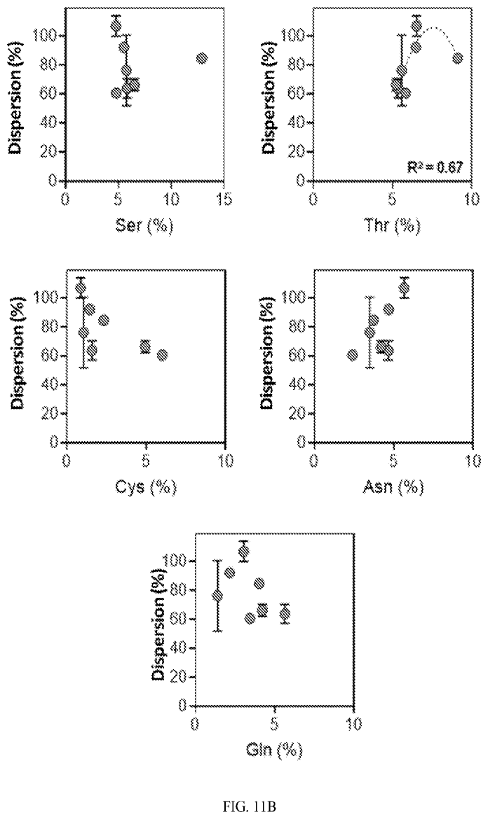

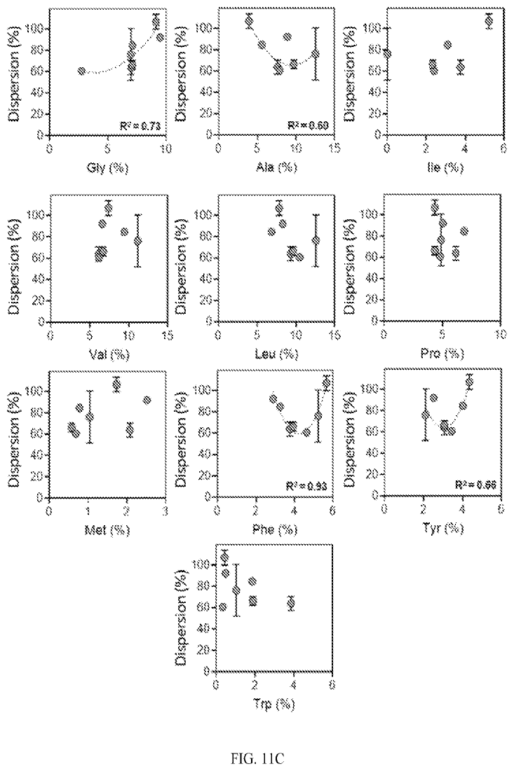

Next, several physicochemical properties of the tested proteins to their phase separation efficiency were compared, with the goal of mechanistically elucidating structural and chemical determinants of PFAS binding activity (FIG. 10A-10F). Given the unique affinity of PFNA, this compound was prioritized for subsequent biophysical experiments. Previous studies suggested that perfluoralkanoic acids decorate proteins via noncovalent hydrogen bonding and electrostatic interactions. Therefore, the hypothesize was that protein size (e.g., molecular weight, solvent-accessible surface area (SASA), hydrodynamic radius) and ionization potential (e.g., isoelectric point (pI)) have predictive capabilities for the potency of PFNA-protein interactions. An inverse relationship between PFNA activity and both protein MW (FIG. 10A) and SASA (FIG. 10B) was observed. This was expected as increasing protein size will correspondingly decrease the surface density of adsorbed PFNA as the equilibrium of the surfactant, which is held at constant concentration in these experiments, moves towards solvation in the bulk solution. However, these factors do not appear to be particularly strong drivers of protein interactions with PFNA, as regression analysis failed to capture most of the variance (R²<0.60).

Unexpectedly, there was a parabolic correlation between PFNA-mediated dispersion efficiency and protein hydrodynamic radius (FIG. 10C). Here, protein dispersion decreased from −100% to 60% as protein radii increased from 2.8 nm to 3.5 nm, with a minimum predicted at 4.8 nm. Beyond this point PFNA activity increased with increasing protein hydrodynamic radius. This suggested that protein size and ionic charge exerted competing effects with respect to the adsorption of the anionic PFNA surfactant. Here, hydrodynamic radius (R_{H}) was inversely proportional to the protein diffusion coefficient (D) following the equation

$${R}_{H} = \frac{{k}_{B} T}{6 πη D} ,$$

where k_{B} was the Boltzmann constant, T temperature, and η solvent viscosity (Reference: 47). In these experiments, T and η remained constant, while D changed as a function of protein ionic mobility (μ) and electrical charge (q) following the relationship

$$D = \frac{μ {k}_{B} T}{q} .$$

Therefore, protein hydrodynamic radius may be counterbalanced by a change in protein surface ionization potential, which may have caused the loss of PFNA-mediated phase separation. That is, differences in protein surface charge may have modulated the avidity of PFNA adsorption to proteins in an inverse manner with respect to size.

To further investigate this, the isoelectric point (pI) of each protein to its PFNA-mediated fluorous dispersion efficiency was compared. Results in FIG. 10D support the hypothesis that protonation state of the protein's solvent accessible amino acids is important to PFNA adsorption. However, the inverted parabolic relationship was again unexpected, as a direct correlation between pI and PFNA avidity had been predicted. This premise was based on the greater propensity for amino acid side chains to become protonated as protein pI increases, and thus obtain a higher net positive surface charge under physiologic conditions. Both amino acid protonation and cationic charge were expected to positively correlate to PFNA avidity, as it favorably mediates hydrogen bonding and electrostatic interactions between PFNA's fluorine and carboxylic groups, respectively, with the protein surface. However, in silico binding studies described later revealed that PFNA favored hydrogen bonding with the protein backbone, rather than amino acid side chains. This suggested that too great of a cationic charge at the protein surface (e.g., high pI) would lead to electrostatic sequestration of anionic PFNA with basic residues, thus inhibiting access to the protein backbone. Conversely, a high anionic surface charge (e.g., low pI) would electrostatically repulse anionic PFNA, again limiting backbone access. A balance between these forces would therefore be ideal, and thus may explain the inverse parabolic trend to the data in FIG. 10D.

Finally, the impact of secondary structure on PFNA-protein interactions was evaluated (FIG. 10E and FIG. 10F). Results showed that increasing α-helicity generally correlated to poorer PFNA dispersion, while the inverse was true for β-sheet propensity. This was likely due to the greater solvent exposure of β-sheets relative to α-helices, where peptide chains in β-sheets were nearly completely extended. The enhanced solvent accessibility of these extended structures has been shown to lead to favored hydrogen bonding between denaturants and the β-sheet backbone of various proteins. While similar mechanisms likely explain the dispersion trends observed in this data, hydrogen bonding between PFNA and proteins does not lead to denaturation, as demonstrated in later structural studies.

To further explore residue specific interactions of PFNA, dispersion activity against the frequency of each amino acid in the protein test set was examined (FIG. 11A-11C). Regression analysis was used to evaluate potential correlations for each comparison, with trends defined by R²≥0.60 highlighted (see dashed lines in FIG. 11B and FIG. 11C). It is important to note that the observed correlations did not explicitly demonstrate causation, and so are used here primarily for hypothesis generation. Notably, the frequency of basic residues (e.g., Lys, Arg, His) did not positively correlate to PFNA avidity, suggesting that electrostatic complexation is not the dominate intermolecular mechanism mediating PFNA-protein interactions. This supported the assertion that PFNA favored hydrogen bonding with the solvent accessible protein backbone. Further affirmation of this was the positive correlation between PFNA activity and protein glycine content, presumably as the lack of an alpha carbon side chain for this residue enabled facile access of PFNA to the protein backbone. Additionally, significant correlations were observed for large aromatic (e.g., Tyr, Phe) and the β-branched amino acid Thr, all of which are residues typically found in R-sheet structures. Further analyses at the functional group level showed no correlation between dispersion efficiency and percentage of amides, carbonyl, alcohol or thiol groups in the tested proteins. Taken together, these results corroborated the assertion that promiscuous hydrogen bonding between PFNA and the solvent accessible backbone of β-sheets dictated PFNA-protein interactions, without apparent amino acid or functional group specificity.

### In Silico Examination of PFNA-Protein Molecular Interactions

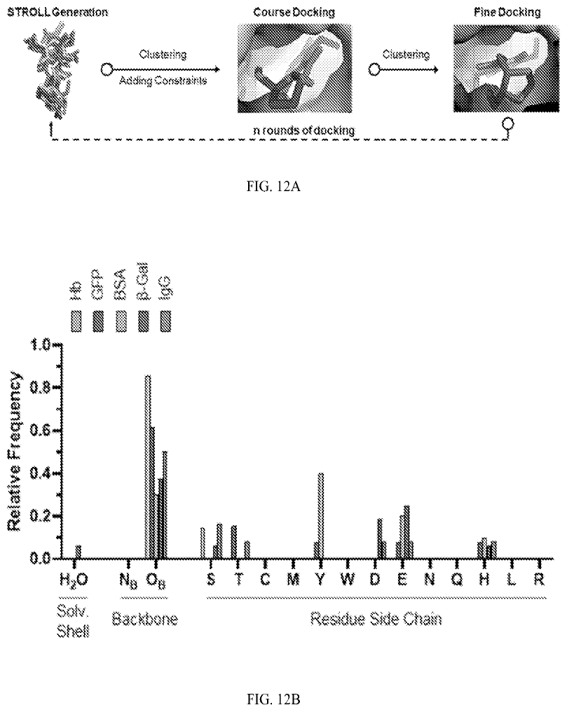

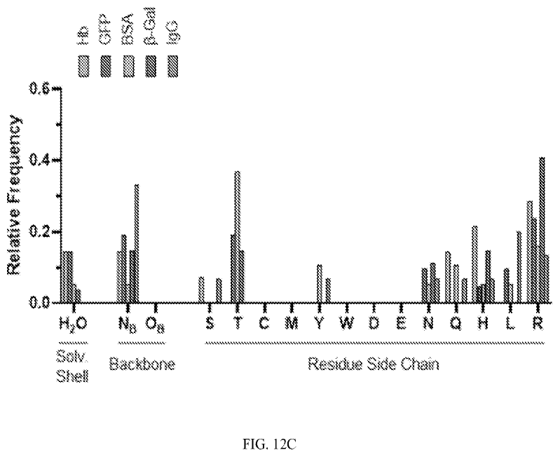

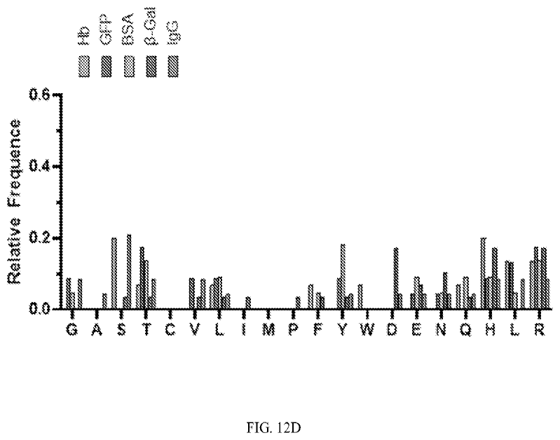

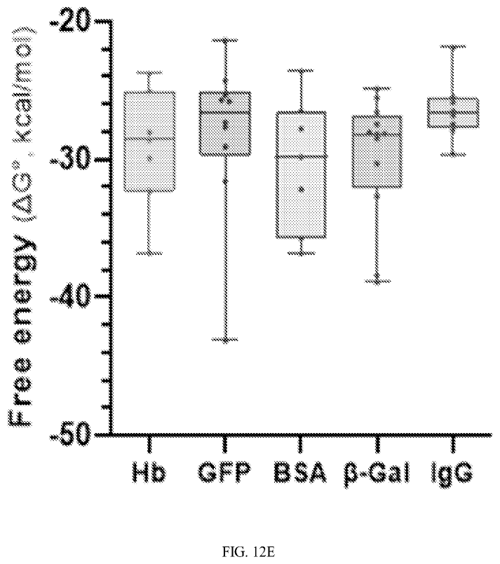

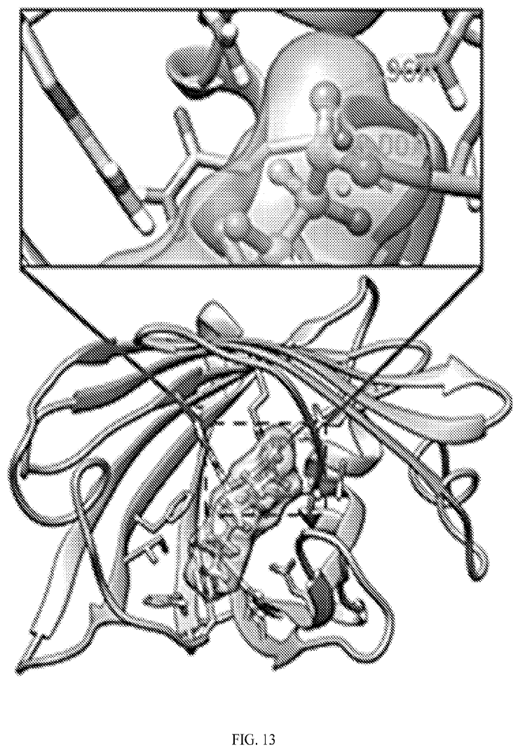

To elucidate the atomistic mechanisms of PFNA-protein interactions, ligand docking simulations were performed using MedusaDock 2.0. The in silico workflow is shown in FIG. 12A. First, a stochastic rotamer library of ligands (STROLL) was generated by randomly rotating rotatable chemical bonds of the PFNA ligand. STROLL rotamers were then clustered and subjected to coarse docking after alignment within a specified protein-ligand binding site for each protein candidate. A total of 7-12 potential binding pockets for the five proteins were analyzed. After coarse docking, the ligand poses were clustered and each of the centroids carried forward to fine docking. Simulations were repeated over n=1000 iterations, and the ligand rotamer with the lowest binding free energy was selected as the final candidate. This process was performed for each binding pocket, and results compiled to evaluate hydrogen bonding frequency of PFNA's carboxyl group (FIG. 12B) and aliphatic fluorines (FIG. 12C) with the solvent shell, backbone, and surface amino acid side chains of each test protein.

Results in FIG. 12B demonstrated that PFNA's carboxyl group (hydrogen bond donor/acceptor) markedly favored hydrogen bonding with protein backbone oxygens (acceptor), that ranged in relative frequencies from 0.30 for BSA to 0.86 for Hb. Apart from tyrosine in the case of BSA, amino acid side chains infrequently formed hydrogen bonds with PFNA's carboxyl group (≤0.20). Results in FIG. 12C showed a complementary analysis for hydrogen bonding of fluorine atoms in PFNA's perfluorinated tail. Compared to oxygen, fluorine is a relatively poor hydrogen bond acceptor due to its high electronegativity and low polarizability. Calculations estimated the strength of a F . . . H bond to be 2-3.2 kcal mol⁻¹, while O . . . H is typically 5-10 kcal mol⁻¹. However, PFNA possesses 8 times the number of fluorine atoms relative to oxygen, suggesting F . . . H bonding may dominate its interactions with proteins. Analysis of F . . . H frequency for PFNA indicated a greater promiscuity compared to O . . . H, with a preference for nitrogen donors present in the protein backbone and side chains of Asn, Gln, His, Lys and Arg amino acids (FIG. 12C). Secondary interactions with water molecules in the solvent shell, and threonine's hydroxyl group, were also observed for select proteins. Finally, parallel analyses predicted hydrophobic contact frequency of PFNA with solvent accessible residues (FIG. 12D), which were generally stochastic with no discernable amino acid preference. Collectively, this data suggested that PFNA's polar head group preferentially adsorbed to the protein backbone to create a fluorine-rich surface coating, with additional stabilization of the fluorinated tail via F . . . H bonding with nearby donors. This yielded conformations with predicted free energies of −21.4 kcal mol⁻¹ to −43.1 kcal mol⁻¹ (FIG. 12E), and hydrogen bonding distances typical for protein-ligand interactions (~2.0 Å-3.0 Å, FIG. 13).

### Conformational Dynamics of PFNA-Coated Proteins

The ability of PFNA to promiscuously adsorb to protein surfaces suggested it may have had similar secondary structure-inducing effects as trifluoroethanol (TFE). TFE is an organofluorine liquid that has long been known to increase α-helix and β-sheet content when used as a co-solvent in aqueous protein samples. While mechanisms explaining this phenomenon are still under investigation, the prevailing paradigm is that TFE molecules coat proteins and preferentially solvate folded regions. This consequently limits accessibility of water to the protein surface, thereby removing alternative hydrogen-bonding partners and providing a low dielectric environment that favors intramolecular hydrogen bonds. These local interactions consequently promote ordered secondary structures.

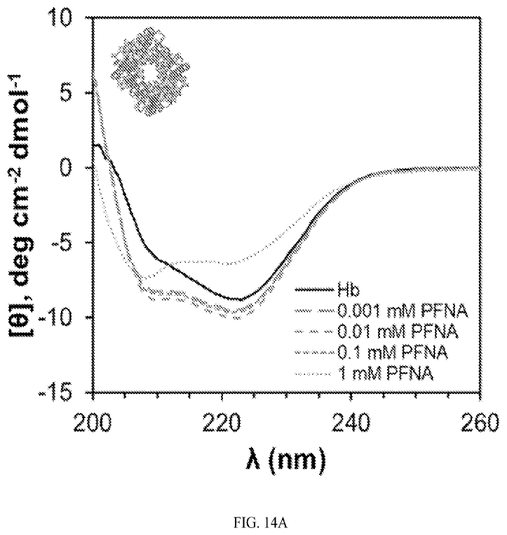

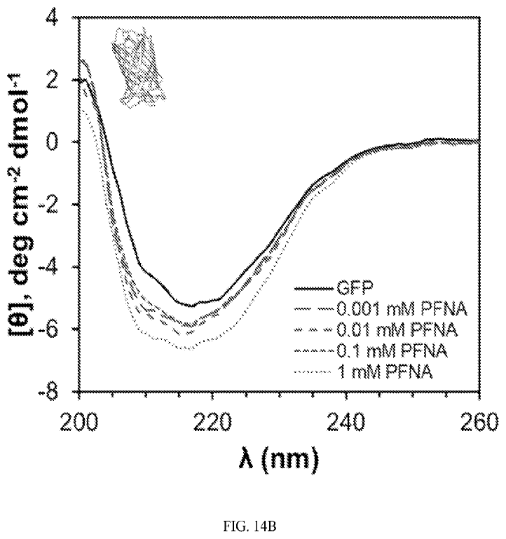

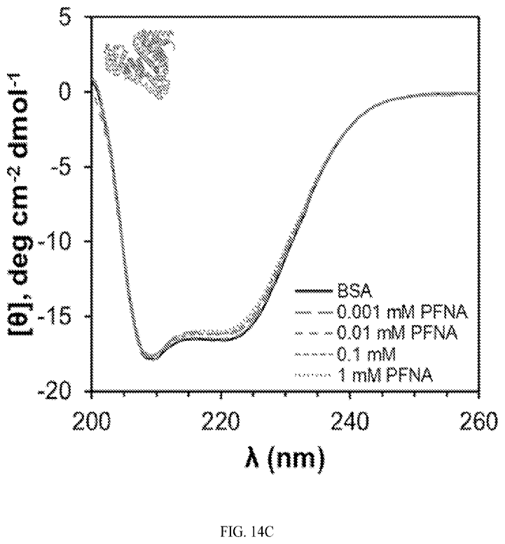

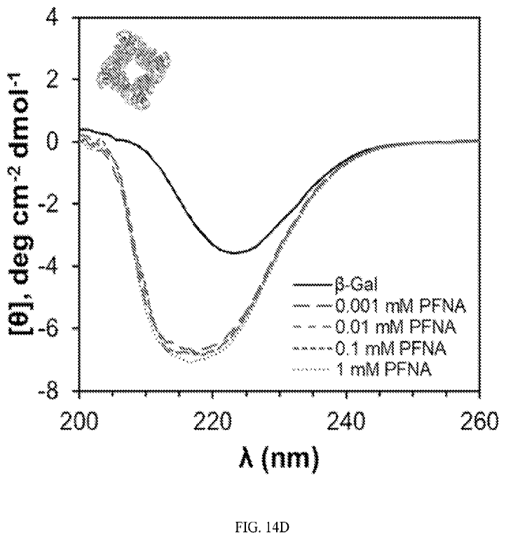

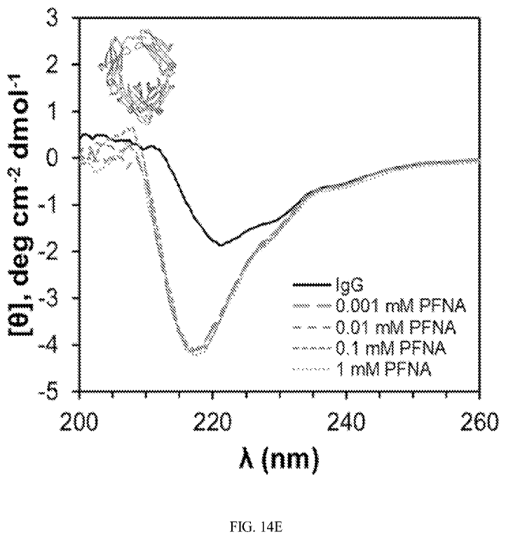

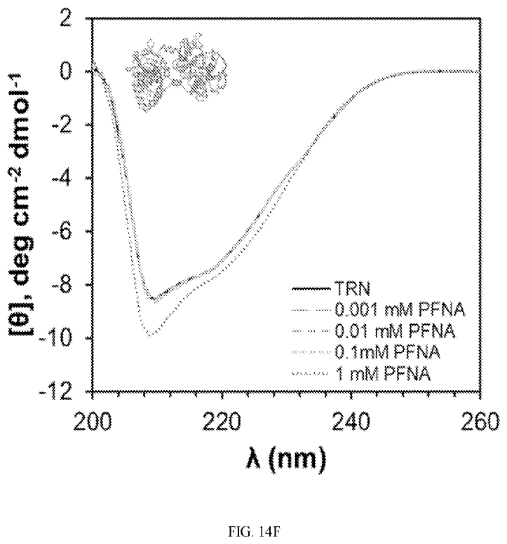

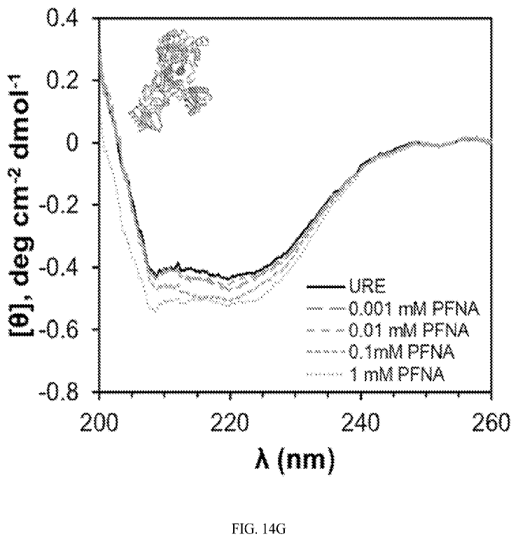

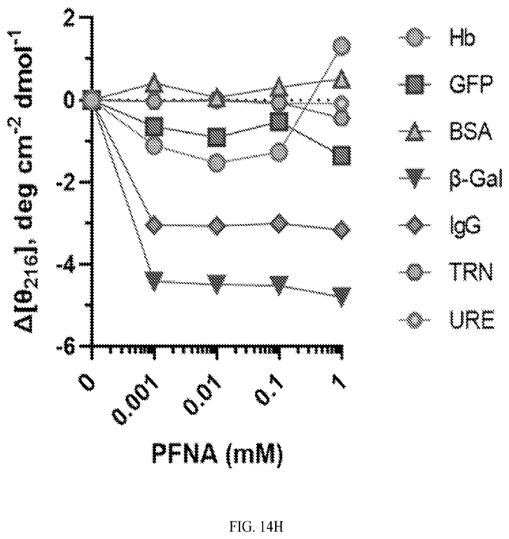

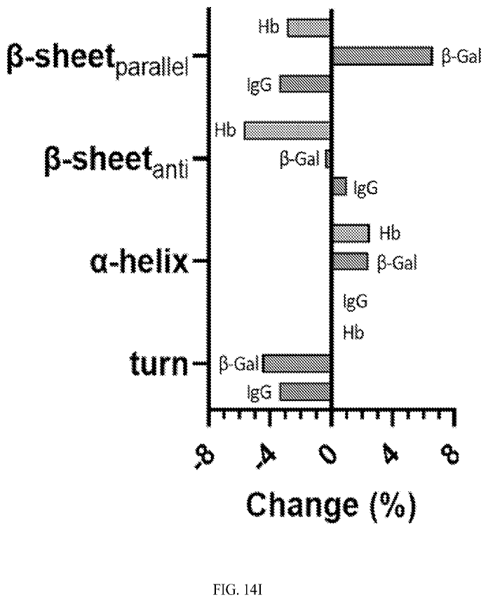

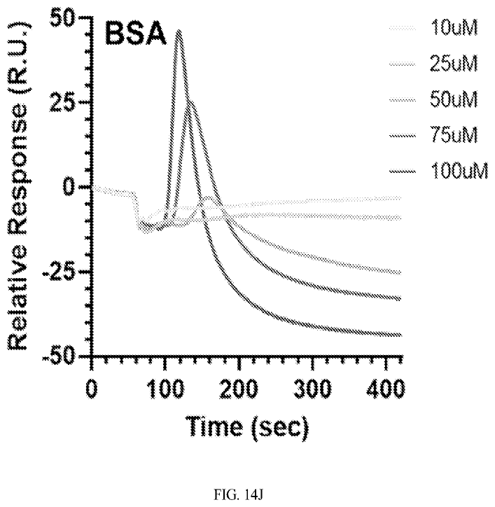

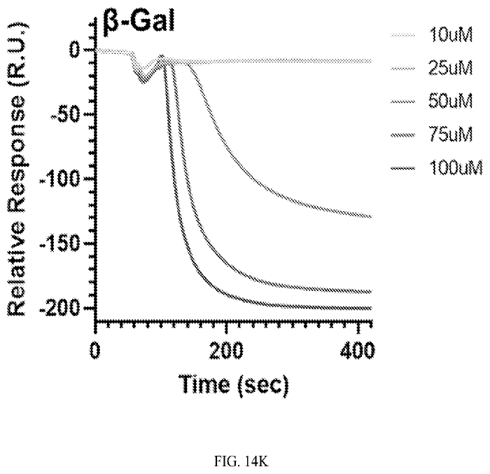

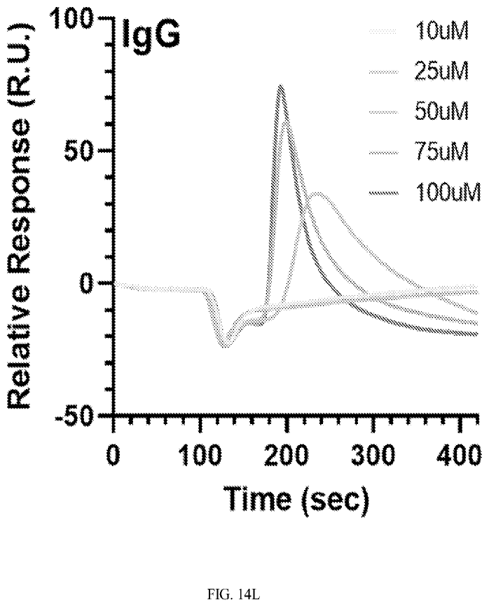

To explore PFNA-mediated changes in protein structure, dose-response circular dichroism (CD) studies were performed (FIG. 14A-14G). Results showed that, like TFE, low PFNA concentrations (0.001-0.1 mM) induced non-native α-helical structure in hemoglobin (FIG. 14A), as demonstrated by the increased intensity of canonical α-helix minima at 208 nm and 222 nm. However, increasing the concentration to 1 mM PFNA resulted in a slight loss of signal, which suggested partial unfolding of Hb under these conditions. GFP showed a slight enhancement of β-sheet structure (θ_{216 nm}) with increasing PFNA concentration (FIG. 14B), while BSA, transferrin and urease structural profiles remained largely unchanged (FIGS. 14C, 9F, and 9G). The most striking effect was observed for β-gal (FIG. 14D) and rabbit serum IgG (FIG. 14E), which both displayed a marked increase in β-sheet content when exposed to 0.001-1 mM concentrations of PFNA. Remarkably, these structural changes endured even at sub-stoichiometric PFNA:protein ratios (see 0.001 mM PFNA in FIG. 11D and FIG. 11E; protein concentration was constant at 0.01 mM). This may suggest that PFNA altered protein conformation and solubility via ensemble effects, rather than a de facto protein-ligand interaction.

To test this assertion, PFNA binding to three exemplary proteins, BSA, β-Gal and IgG, was studied via isothermal titration calorimetry (ITC) and surface plasmon resonance (SPR) techniques. ITC showed no significant evolution of heat for all three proteins in the presence of PFNA, further supporting the hypothesis of promiscuous interactions of the ligand. SPR analysis yielded similar results, with sensorgrams (FIGS. 14J-14L) that diverged from a simple 1:1 ligand-protein model. Specifically, a slow, dose-dependent reduction in the signal was observed after analyte injection, which upon reference correction yielded negative responses. While non-specific binding of PFNA to the reference cell, or other related artifacts, could not be conclusively ruled out as the cause of the negative signals, careful inspection of the reference surface and inclusion of multiple control samples suggested an alternative explanation. Prior studies have reported negative SPR signals from specific receptor-ligand interactions in which analyte binding resulted in conformational change of the target protein. Similar phenomenon may explain the negative signals of the SPR data and further support the ability of PFNA to modulate the conformational plasticity of adsorbed proteins.

Additional ¹⁹F nuclear magnetic resonance (NMR) experiments demonstrated a broadening of PFNA's peaks upon interaction and chemical exchange with two exemplary proteins (BSA & TFN). Interestingly, fluorine groups located at the far end of PFNA's perfluorocarbon tail (distal to the polar head) showed the greatest effects, suggesting reversible binding at these positions. This further supported the conclusion that, like TFE, adsorption of PFNA to protein surfaces aligned the fluorinated tail parallel to the solvent accessible exterior, with further stabilization via F . . . H bonding with surface N—H groups (FIG. 12, FIGS. 12A-12E, and FIG. 13). This ultimately generated the fluorine rich coating that altered water accessibility and promoted secondary structure.

Although PFNA and TFE share a similar capacity to induce non-native secondary structure, PFNA was markedly more potent. Prior studies have shown ≥15 vol % of TFE in water, which represents a 2M total concentration, was required to induce protein structural transitions. The data presented here indicated that 0.001 mM PFNA was sufficient to induce ordered non-native structure for many of the tested proteins (see FIGS. 14A-14I), a 10⁶ increase in potency relative to TFE. In addition, PFNA appeared to favor modulation of 1-sheet structures over α-helices, which can be interpreted by monitoring changes in both ellipticity at 216 nm (FIG. 14H) and percentage of secondary structural motifs (FIG. 14I) relative to the native protein. Sensorgrams (FIGS. 14J-14L) did not appear to represent a simple 1:1 ligand-protein model. The dose-dependent reduction in the signal after analyte injection may have been due to conformational change of the target protein, as has been observed in prior studies.

This was contrary to the behavior of TFE, which generally increased the population of α-helices in exposed proteins. The prior observation that PFNA preferentially associated with the solvent accessible backbone of β-sheets (FIG. 10F) may explain this difference. With respect to the potency of PFNA, this was likely due to the greater avidity of this molecule for proteins when compared to less fluorinated amphiphiles (FIGS. 8A-8G), like TFE. More stable association and greater fluorine content at the protein surface following PFNA complexation, relative to shorter chain PFAS compounds, would lead to increased water displacement and favored intrapeptide hydrogen bonding. Importantly, since PFNA interacted only weakly the protein backbone, hydrophobic interactions were not disrupted. As a result, PFNA promoted protein stability rather than inducing denaturation.

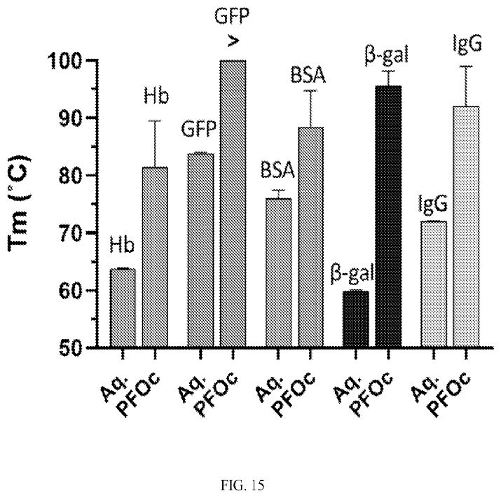

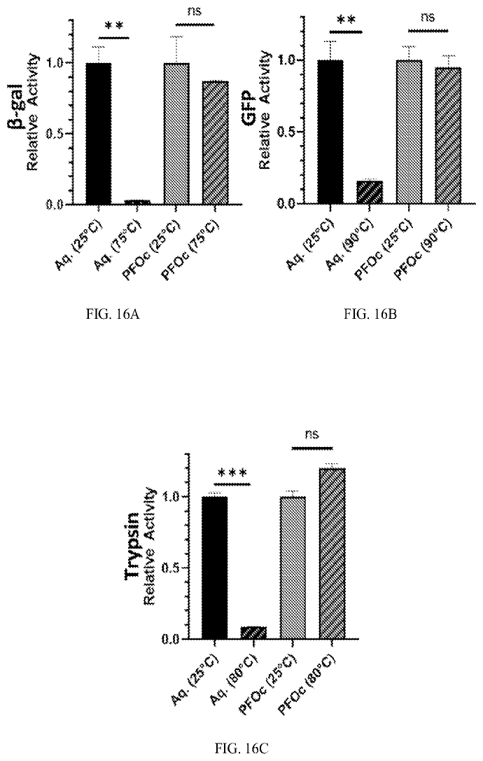

PFNA enhanced thermal stability in PFOc Differential scanning calorimetry (DSC) measurements were performed on 5 test proteins dissolved in either PBS (Aq.) or Perfluorooctane (PFOc). PFNA dispersant was added only for the PFOc conditions. DSC was performed at 2 °C/min ramp from 40 °C-100 °C. Melting temperature (Tm) was determined based on positive inflection of heat capacity curve, indicating protein unfolding (FIG. 15). Change in melting temperature (ATm (°C)) represented the difference in Tm between aqueous (Aq.) and PFOc data. Tm for GFP in PFOc could not be achieved at the maximum temperature of 100 °C for the DSC experiments. Each protein was dissolved in PBS (Aq.) or PFNA/PFOc mixture. Dissolved proteins were heated to 75 °C (β-gal), 90 °C (GFP) or 80 °C (Trypsin); treatment temperatures were determined based on aqueous melting temperatures for each protein. Positive controls were maintained at room temperature (25 °C). After heating at indicated temperature for 30 minutes, proteins were extracted by addition of an equal volume of PBS and vortexing. Bioactivity was evaluated then via fluorescence measurements for GFP, or using colorimetric substrate conversion assays (ONPG for β-gal, BAEE for Trypsin). The results are available in FIGS. 16A-16C. This procedure demonstrated that proteins dispersed in PFOc can be readily extracted into physiologic solutions. This data supports the proof-of-principle for the ability of PFOc protein formulations delivered to animals in in vivo experiments to distribute systemically and remain bioactive.

### In Vitro Bioeffects of PFNA-Protein Complexation

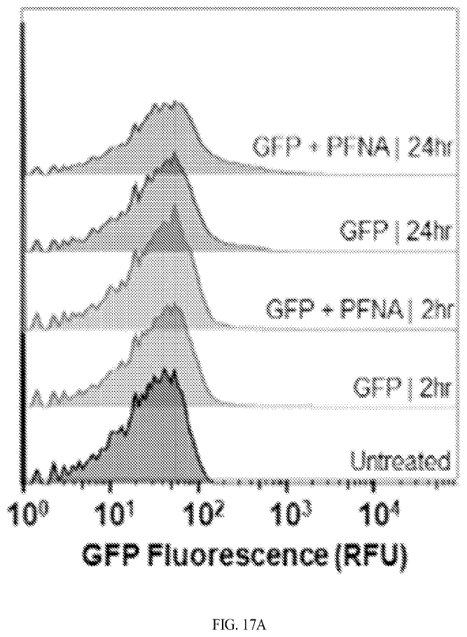

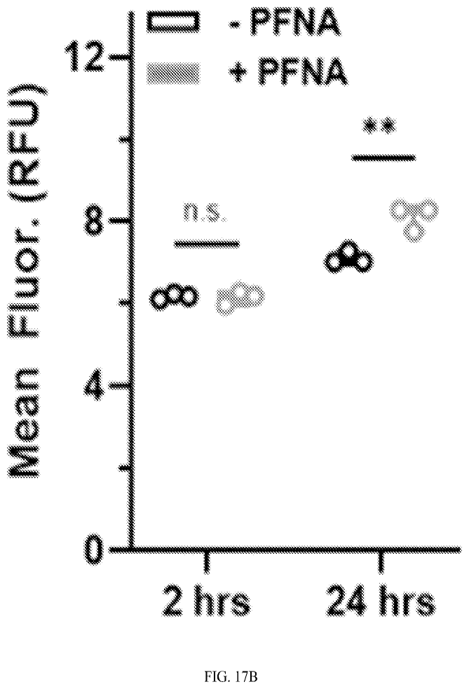

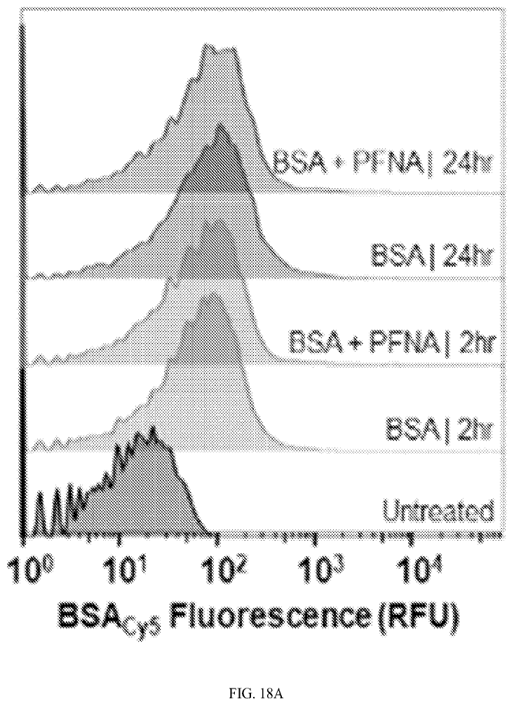

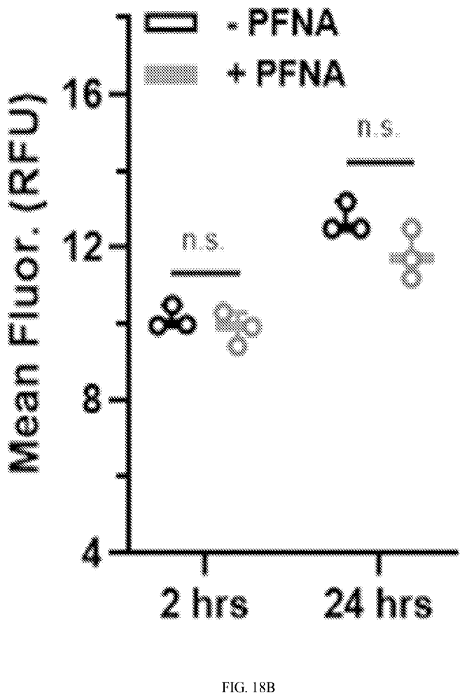

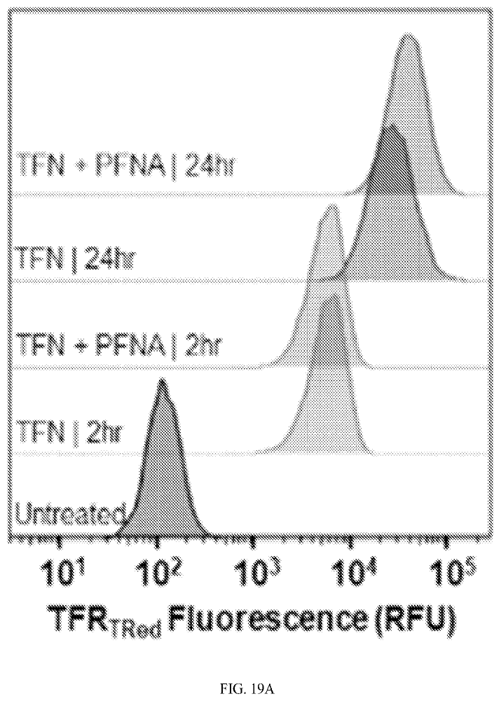

Multiplexed in vitro assays were employed to probe the influence of PFNA interactions on protein uptake and transport in cells. A549 and PC9 human lung cells were prioritized for these assays given that pulmonary tissue is a primary site of perfluoroalkyl bioaccumulation in humans (Reference: 63). Before initiating experiments, cellular toxicity assays established a maximum tolerated dose of PFNA to avoid confounding protein transport experiments with substantive cell death. Protein uptake was then studied using green fluorescent protein (GFP) and bovine serum albumin (BSA_{Cy5}) as model biologics that employ non-specific pinocytic mechanisms for cellular entry. FIGS. 17A-17B and FIGS. 18A-18B showed that PFNA adsorption to GFP and BSA, respectively, led to a nominal increase in uptake only for GFP at the 24-hour exposure time point. These results were compared to uptake of Transferrin, a protein that enters cells via receptor-mediated endocytosis (FIGS. 19A-19B). Results showed a marked increase in Transferrin internalization in the presence of PFNA, with a 43% increase in cellular fluorescence compared to the native protein after 24 hours of incubation.

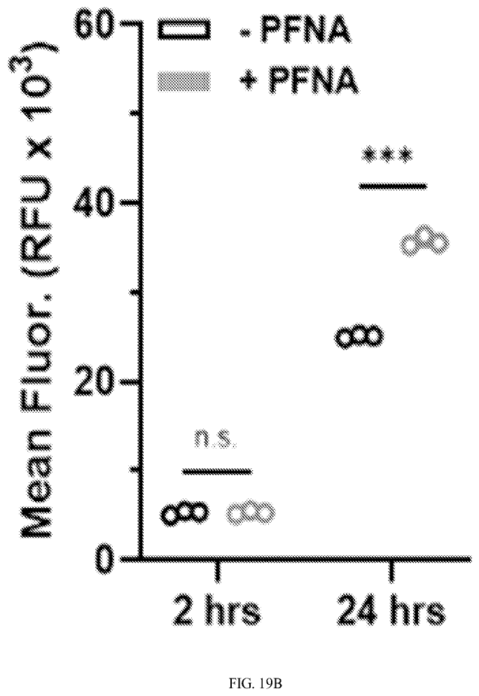

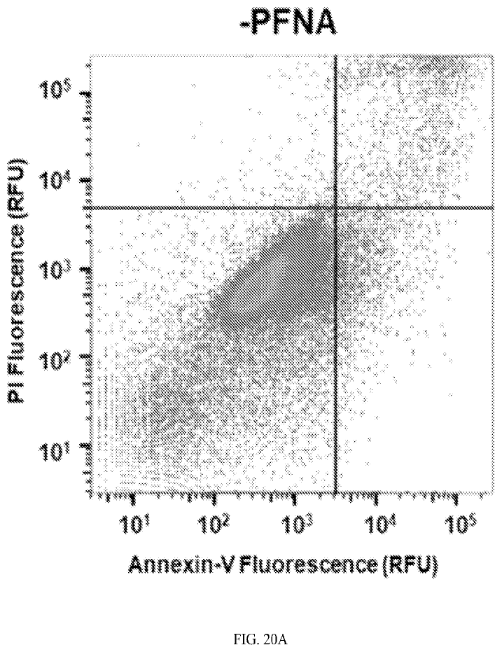

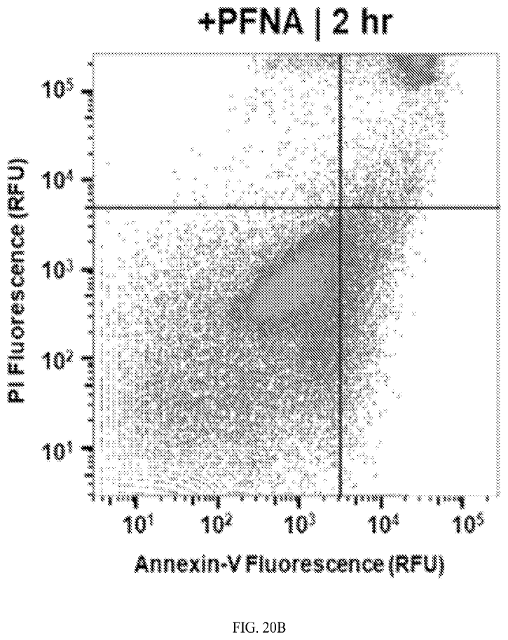

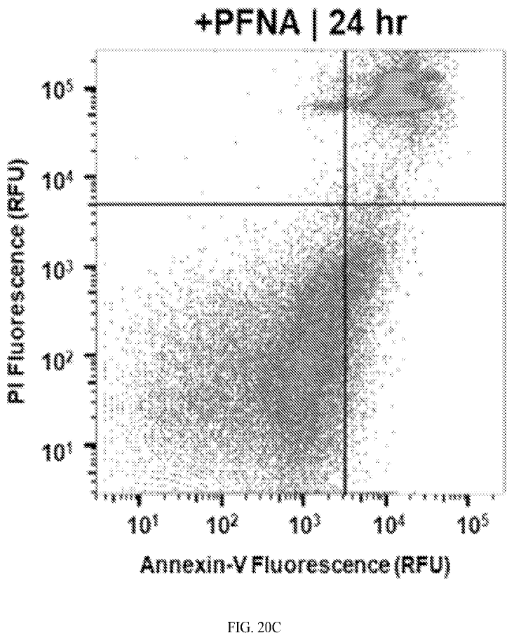

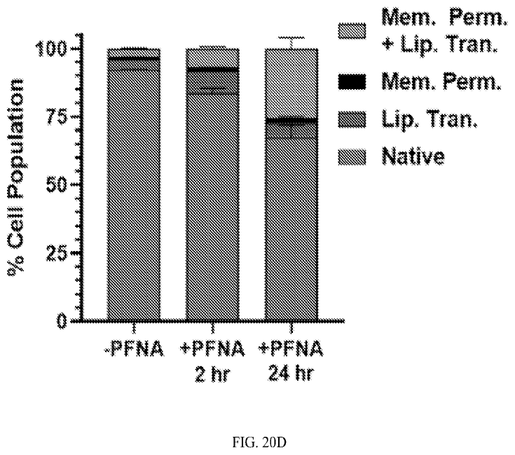

The ability of PFNA to modulate protein uptake may be explained by the reported anchorage of fluorinated amphiphiles within cell membranes to alter lipid bilayer fluidity and liquid-crystalline phase transitions. To test this assertion, and inform the interpretation of the protein uptake results, the annexin V (AV)/propidium iodide (PI) assay, commonly used to study apoptosis, was repurposed to explore membrane dynamics following PFNA exposure (FIGS. 20A-20D). Here, AV bound phosphatidylserine lipids exposed via inner-to-outer leaflet translocation, while PI positively labeled cells with permeabilized membranes. Comparing dye uptake at early (2 hour) and late (24 hour) PFNA exposure times allowed interpretation of lipid translocation (AV⁺, PI⁻), membrane permeabilization (AV⁻, PI⁺), both (AV⁺, PI⁺), or neither (AV⁻, PI⁻). Results showed that at 2 hours of exposure lipid translocation (AV⁺, PI⁻) was elevated, and at 24 hours both lipid translocation and membrane permeability (AV⁺, PI⁺) were increased. Prior studies indicating perfluoroalkyl surfactants increased the disorder and fluidity of lipid bilayers suggested that these results may be explained by similar mechanisms. Specifically, integration and self-assembly of PFNA within cell membranes may have rearranged lipids in the asymmetric bilayer to modulate lipid packing and fluidity, subsequently promoting the internalization of GFP and Transferrin. BSA internalization appeared to be less affected by these phenomena, likely due to the weak interactions of this protein with PFNA (FIG. 14C).

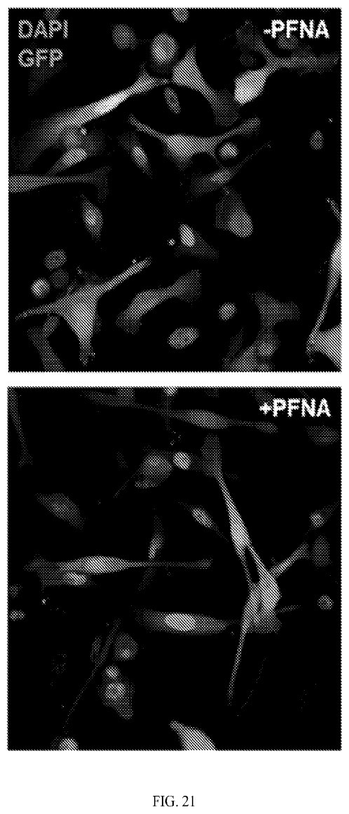

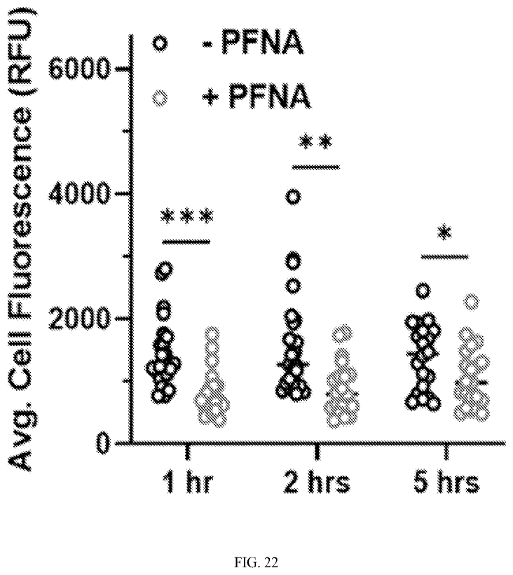

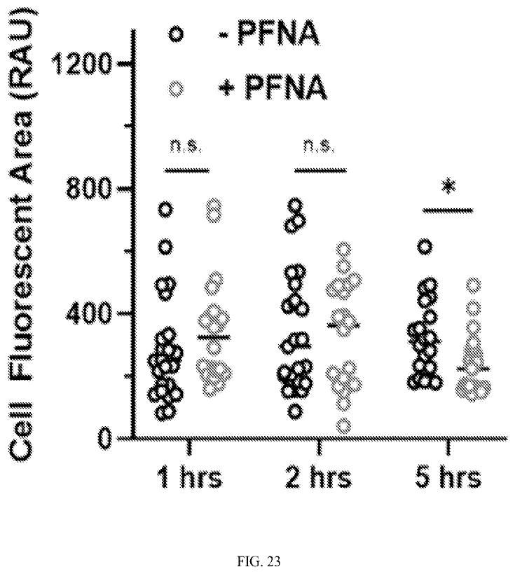

Imaging studies were performed to interpret the effect of PFNA exposure on the transport of endogenous proteins in cells. For these experiments, stably transfected eGFP-PC9 lung cells were prepared to track the time dependent sub-cellular localization of eGFP as a model protein target (FIGS. 21, 17, and 18). Results showed a significant reduction in eGFP expression in PFNA-treated cells, particularly at acute exposure times (see 1 hour in FIG. 22). This suggested that the perfluoroalkanoic acid may have broadly suppressed protein synthesis in treated cells. In contrast, distribution analyses indicated overall intracellular protein localization remained relatively unchanged (FIG. 23).

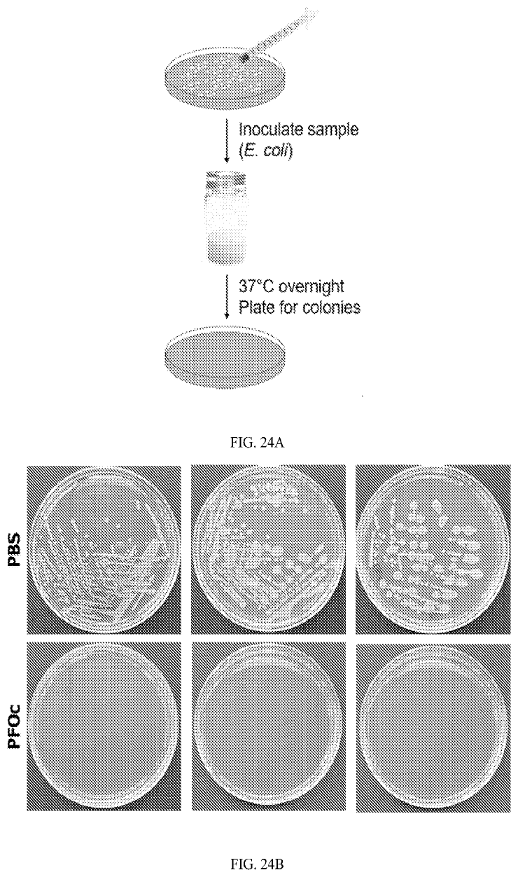

To model bacterial contamination of samples a hypodermic needle was streaked across a lawn of plated E. coli (FIG. 24A). The contaminated needle was then dipped into PBS or PFOc (containing PFNA) samples with dispersed BSA protein to contaminate, and samples incubated at 37 °C overnight to allow for bacterial growth. An aliquot of the solution was then streaked onto LB-agar plates and incubated at 37 C overnight to allow for bacterial colony formation. Visible E. coli colony growth from PBS samples indicated contamination, while PFOc remained sterile (FIG. 24B).

### Conclusion

Organofluorines possess attractive chemical and biological properties that have allowed them to make paradigm shifts in the design of pharmaceuticals and biomaterials. For example, the introduction of fluorine atoms into amino acids has opened a vast new chemical landscape with which to alter the folding, stability, oligomerization propensity and bioactivity of peptides and proteins. However, chemical synthesis of highly fluorinated amino acids requires harsh reaction conditions, and to date it remains difficult to biosynthetically prepare highly fluorinated proteins. Here an alternative strategy is presented of non-covalently adsorbing fluorinated small molecules to proteins to alter their folding and phase separation properties. The unique benefit of this approach is its promiscuity, where the results showed that privileged perfluoroalkyls can interact indiscriminately with various protein classes. This suggested that the strategy may be applied beyond proteins to alter the organization and assembly of other biomolecules, including lipids and carbohydrates. More broadly, these studies help to establish design principles governing how fluorine tags can be used to guide the folding, stability, and supramolecular assembly of biologics to form new bioinspired materials. Defining a comprehensive mechanistic understanding of perfluoroalkyl interactions with proteins, and their effects on cells, also informs on potential modes of toxicity and guides the rational design of new fluorinated adjuvants with desirable functional properties for drug discovery and nanomedicine applications.

### Example 2: Studies on Additional Perfluoroalkyl-Protein Compounds

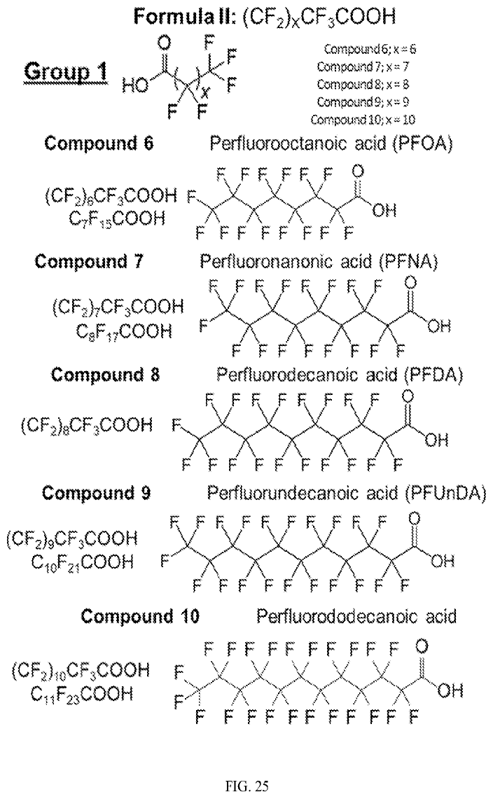

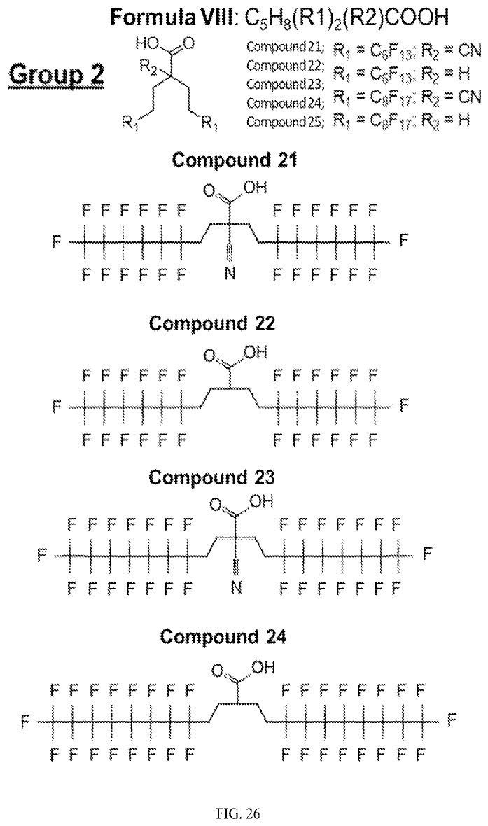

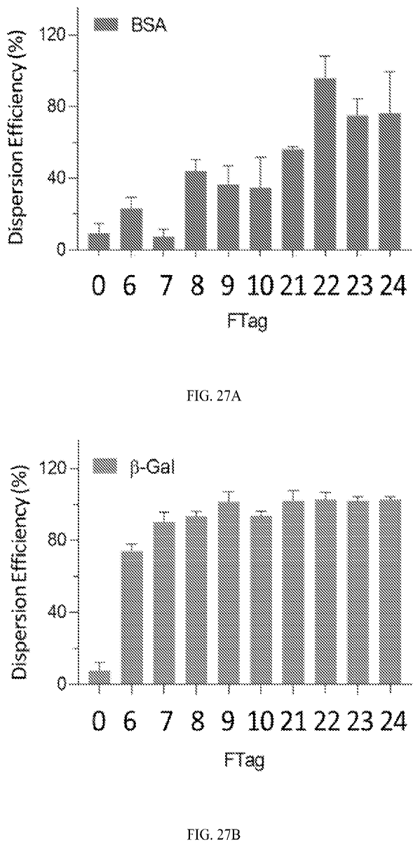

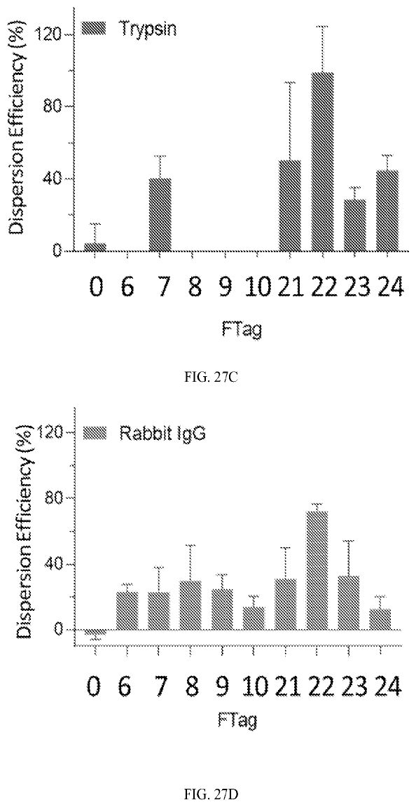

Additional perfluorinated small molecule compounds containing FTags were selected (FIG. 26) and tested against select first generation perfluorinated small molecule compounds (FIG. 25). The second-generation compounds each contained an FTag that contained two R₁ groups and one R₂ group. The R₁ group was C₆F₁₃ for compound 21 and compound 22 and was C₈F₁₇ for compound 23 and compound 24. The R₂ group was CN for compound 21 and compound 23 and was H for compound 22 and compound 24. The FTag compounds were dissolved in PFOc (perfluorooctane) and added to dried protein to achieve a final protein:FTag molar ratio of 1:1000 (1 uM:1 mM). Solutions were then vortexed and sonicated for predefined periods to disperse initial aggregates and rotated overnight at room temperature to fully mix. Solutions were then centrifuged to remove undispersed protein precipitates, supernatants were transferred to a clean well, and PFOc was evaporated at 37 °C overnight. The resulting protein residue was then dissolved in PBS and a Bradford colorimetric assay was performed to quantify protein concentrations. Dispersion efficiency was determined relative to positive controls of protein in PBS. The data is available in FIGS. 27A-27D. Note that the 7 compound is PFNA. The second generation perfluorinated small molecule compounds had trending greater dispersion efficiency when mixed with BSA compared to the first-generation compounds (FIG. 27A). Dispersion efficiency was about the same between the first and second generation perfluorinated small molecule compounds when mixed with β-gal (FIG. 27B). The second generation perfluorinated small molecule compounds had similar or greater dispersion efficiency when mixed with trypsin compared to PFNA (FIG. 27C). Dispersion efficiency was about the same between the first and second generation perfluorinated small molecule compounds when mixed with rabbit IgG (FIG. 27D).

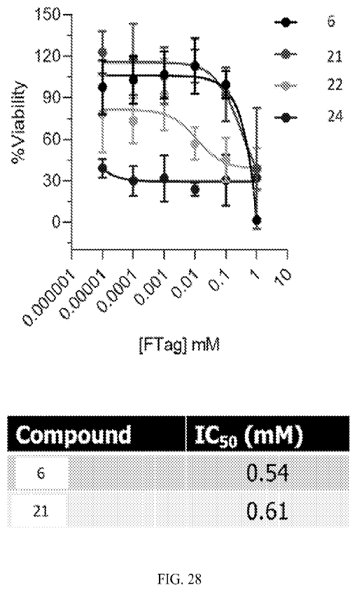

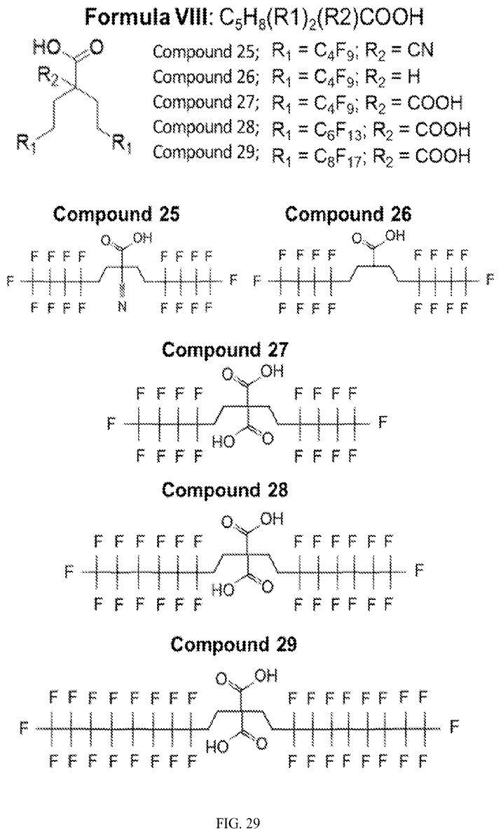

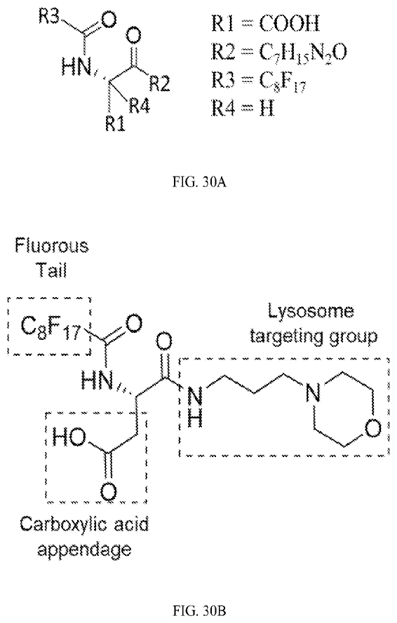

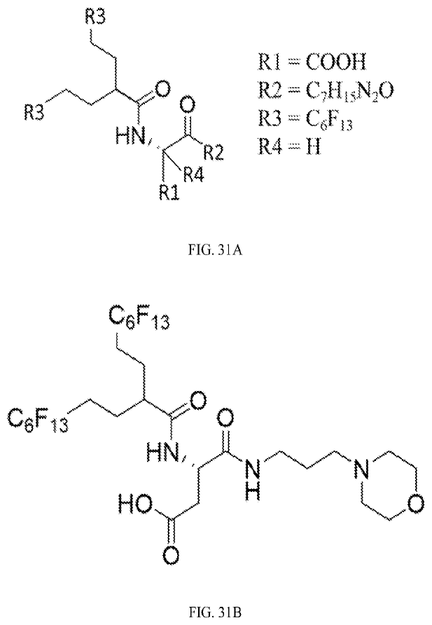

Next, viability assays were performed by seeding HepG2 (liver cancer cell line) onto 96 well plates and allowing the cells to adhere overnight. DMEM cell culture media containing various concentrations of each FTag compound were then added, and the plates were incubated at 37 °C overnight. The supernatant was then removed and MTT viability dyes were added to each well and allowed to convert for 2 hours. Dye absorbance for each condition was then compared to positive (20% DMSO) and negative (blank media) controls to calculate % viability (FIG. 28). Overall, the cells exposed to the 7 and 21 compounds showed the highest viability, with IC₅₀ values of 0.54 and 0.61 mM, respectively.

### Example 3: In Vivo Assessment of Equivalency and Toxicity

Two groups (n=5) of C57BL/6J mice received 200 μL of 10 μM solutions of β-Gal that was either 1) prepared in sterile PBS or 2) dissolved in PFOc (1 mM PFNA) and extracted into sterile PBS. Due to the large solution volumes given (150 μL, ~15% of mouse blood volume), PFOc dispersed proteins were extracted into sterile saline before injection to avoid hyponatremia. Samples were administered by tail vein injection.

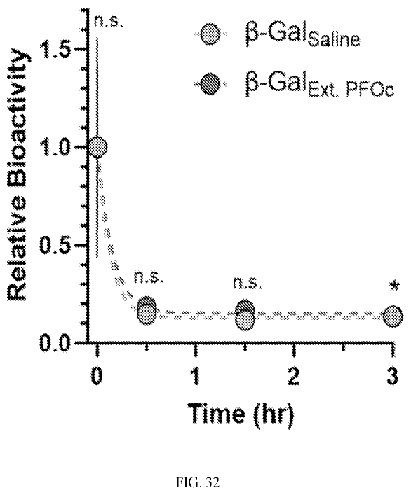

At a series of time points, mice were sacrificed, and blood samples collected via cardiac puncture. Bioactivity and bioavailability of β-Gal was determined by conversion of the fluorescent substrate, 4-Methylumbelliferyl-α-D-galactopyranoside (β-GAL). Whole blood collected in a serum tube was centrifuged at 2000 rcf for 4 minutes to extract serum containing β-Gal protein. 20 μL of the serum was then mixed with 180 μL μ-GAL (for a final substrate concentration of 1 μM). Samples were quickly pipette mixed and incubated in the dark at room temperature for 5 minutes. Fluorescent intensity was measured with λₑₓ=360 nm and λₑₘ=440 nm. The background fluorescent intensity from serum was determined from serum dilution in PBS without μ-GAL substrate. Serum half-life of β-Gal delivered from PFOc dispersion, measured using a fluorescent substrate conversion assay, was shown to possess a nearly identical half-life (t_{1/2}=7.9 min.) to native β-Gal administered in saline (t_{1/2}=6.6 min.) (FIG. 32). These results demonstrated that fluorous dispersion did not alter the bioactive function or pharmacokinetics of the host protein.

To assess acute toxicity, animals were intravenously administered solutions of β-Gal either prepared in sterile PBS or dissolved in PFOc (1 mM PFNA) and extracted into sterile PBS, as described above. After 24 hours, mice were sacrificed, and blood was collected via cardiac puncture. A minimum of 100 μL of whole blood was added to EDTA for complete blood count. The remainder was centrifuged (1000 rcf, 10 minutes) to isolate serum for chemistry analysis. Tissues (lungs, livers, kidneys, and spleens) from euthanized animals were collected and stored in 10% buffered-formalin immediately after euthanasia. Four 5 μM sections from each organ were mounted onto glass slides and stained with Hematoxylin and Eosin (H&E). Four random fields in each section were examined under a microscope at 5× and 40× magnification. Tissue relevant clinical features were compared across samples. Specifically, lungs were examined for infiltration of poly and mono nuclear cells, signs of hemorrhage, and perivascular infiltration. Kidneys were examined for signs of necrosis, cellular infiltration, and hemorrhage. Livers were examined for hepatic cell necrosis, inflammation, and hemorrhage. Spleens were examined for changes in white and red pulp structure, as well as signs of abnormal cellular infiltration.

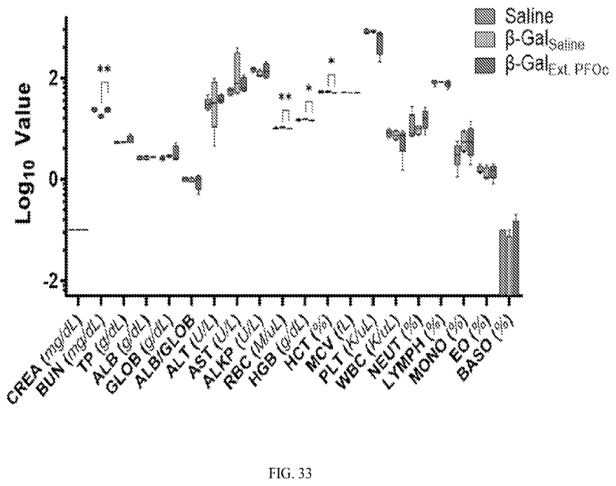

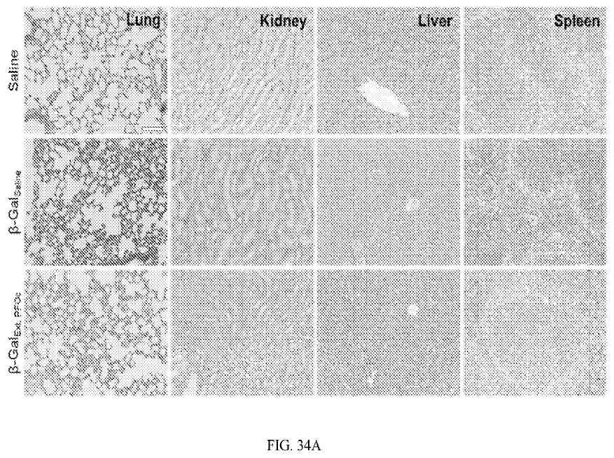

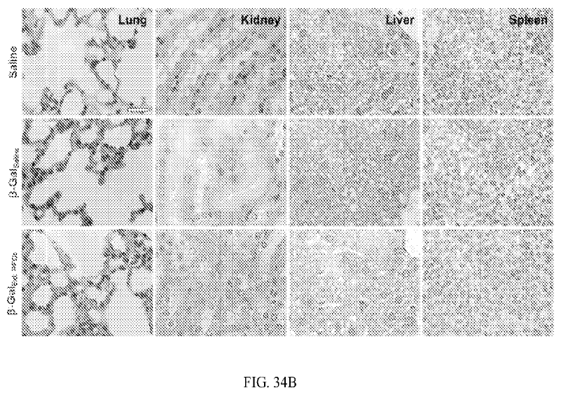

Serological hematologic, renal, and hepatic toxicology screens showed statistically significant changes in blood urea nitrogen (BUN), red blood cell count (RBC), hemoglobin (HGB), and hematocrit (HCT) between β-Gal delivered from PFOc versus saline (FIG. 33). However, none of these markers were statistically different between PFOc β-Gal and the sham saline injection control, suggesting these small changes were not clinically meaningful. This is corroborated by histologic organ analyses that did not identify signs of necrosis, cellular infiltration, inflammation, or hemorrhage in lung, kidney, liver, and spleen tissue excised from treated animals (FIGS. 34A-34B). Collectively, these results suggest that the fluorous media used in protein dispersion formulations are unlikely to induce acute toxic side effects.

### Example 4: Development and Validation of Optimized Perfluorochemical Dispersion Reagents for Extremophilic Protein Formulations

### Introduction

As described herein, perfluorochemical additives allow proteins to disperse in non-aqueous perfluorocarbon (PFC) liquids, maintaining their structure and function even at high temperatures, and providing resistance to bacterial, fungal, and proteolytic contamination. Disclosed herein is an optimized family of perfluorochemical dispersion reagents with enhanced PFOc solubilization efficiencies and biocompatibility. Using a multi-faceted approach, compound PD-7 was demonstrated to show superior performance, sterility, and safety compared to PFNA. Notably, PD-7 separates from the protein surface in physiological solutions, reducing the risk of tissue bioaccumulation of fluorinated reagents, addressing concerns over the toxicity of perfluoroalkyl substances.

### Selection of Protein Dispersants Based on Previous Structure-Activity Research

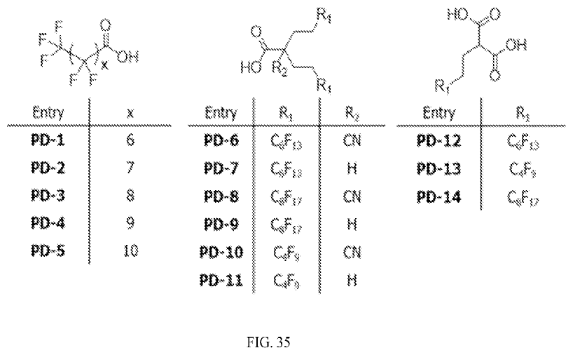

Based on prior structure-activity research described above, key design criteria guided the following design and development of protein dispersants. Three compound families were developed that included rational combinations of mono- or di-acids, as well as single or bivalent perfluorinated tails (FIG. 35). The single perfluorinated tail containing protein dispersants (PD-1 to PD-5) were procured commercially, whereas bivalent perfluorinated derivatives (PD-6 to PD-11) were synthesized through modification of reported protocols. Subsequent acidic hydrolysis and decarboxylation of these dispersants was performed to isolate PD-7, PD-9, and PD-11 in good yields. Similarly, monoalkylation of diethylmalonate with (perfluoroalkyl) ethyl iodides followed by basic hydrolysis gave diacids PD-12 to PD-14 in quantitative yields.

### Comparative Evaluation of Fluorous Dispersion in Protein Compounds

These compounds were screened to evaluate their ability to disperse four model proteins, bovine serum albumin (BSA), β-Galactosidase (β-Gal), rabbit serum immunoglobulin (IgG), and bovine trypsin into the PFOc solvent. These proteins were selected for their diversity in molecular weight and metabolic function (e.g., enzymes, antibodies, and carrier proteins) to demonstrate the universal nature of this fluorous dispersion methodology. To determine dispersion efficiency, 200 μL of perfluorohexane (PFHx) solvent with 1 mM PFNA additive was added to each of lyophilized protein stock in a 1.5 mL centrifuge tube. The samples were then briefly vortexed/sonicated then constantly mixed overnight to ensure proper fluorine dispersion. After that, samples were centrifuged at 1950 g for 5 minutes to remove non-dispersed portion, and 100 μL supernatant of dispersed protein sample was transferred into each well of 96-well plate. PFHx solvent was let evaporate at 37 °C for two hours. The remaining protein residue was redissolved in 100 L PBS, and an equal volume of Coomassie solution was added to the sample. In addition, protein prepared in PBS and PFHx (without PFNA) were also identically processed and included as positive and negative control. Lastly, the amount of protein was quantified by taking absorbance of converted Coomassie solution at 595 nm. Dispersion efficiency was calculated as follows:

$$Dispersion Efficiency ( \% ) = \frac{( {Absorbance}_{Sample} - {Absorbance}_{Negative control} )}{( {Absorbance}_{Positive control} - {Absorbance}_{Negative control} )} * 100 \%$$

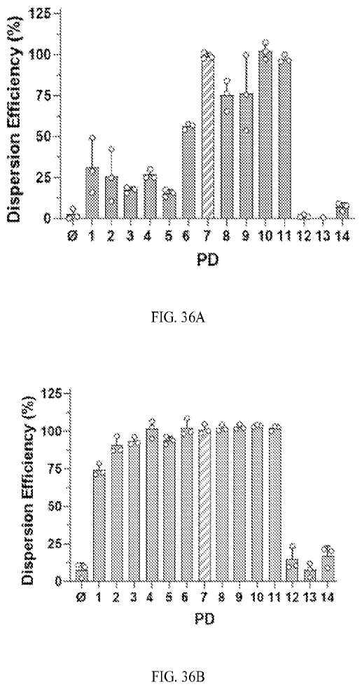

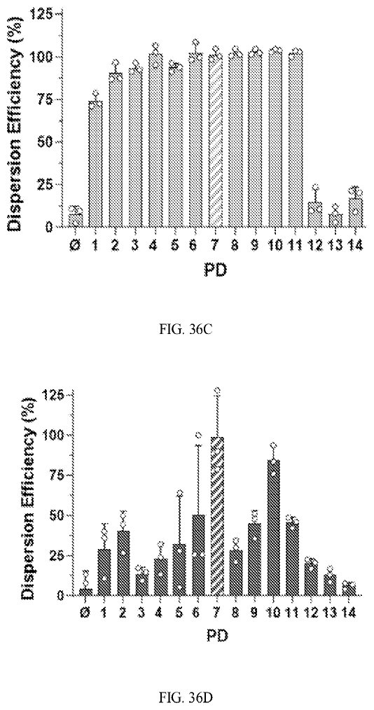

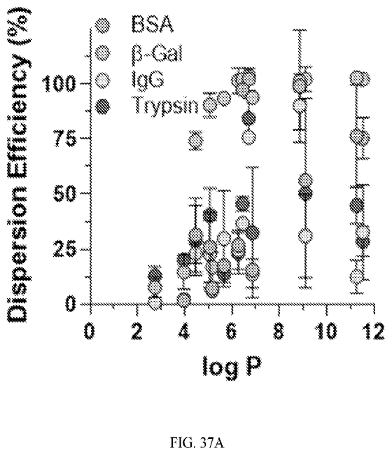

PD-7 (FIGS. 36A-36D) displayed dispersion efficiencies of 90%-100%, outperforming all other PDs in the test set. PD-10 was a notable secondary candidate, achieving 80%-100% dispersion efficiencies. Importantly, the lead compound from previous screens, PFNA (referred to as PD-2 in this study), generally did not achieve >50% dispersion of the tested proteins in PFOc. The notable exception was β-Gal, which showed nearly quantitative dispersion across candidates PD-2 through PD-11 (FIG. 36B). The most likely explanation for this is that the high molecular weight of the protein tetramer complex (~520 kDa) provides an abundant surface area for complexation of the soluble dispersants. Interpreting structure-function-performance relationships from these results suggests that divalent fluorinated tails, particularly C₄F₉ and C₆F₁₃, outperform monovalent analogues with similar fluorine content. Likewise, mono-acid dispersants perform better than di-acids, most likely due to the improved solubility of mono-acid derivatives in PFOc.

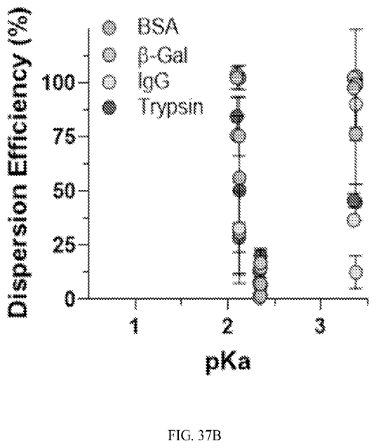

To better understand the physiochemical determinants of dispersant activity, the partition coefficient (log P) and dissociation constant (Kₐ) of each PD was compared to its dispersion efficiency (FIGS. 37A-37B). This analysis showed that a dispersant log P≥7 generally maximizes the dispersion efficiency of a given protein. No clear correlation was observed between carboxy pKa and dispersion efficiency. Taken together, the data suggests there are three properties important to PD-protein PFOc dispersion: 1) divalent fluorinated tails outperform monovalent tails; 2) mono carboxylic acid amphiphiles are preferred over di-acids; and 3) dispersion efficiency increases with PD log P up to a value of 7, likely due to improved solubility of the dispersant in the bulk perfluorocarbon solvent.

### Toxicity Screening of Compounds and Selection of Lead Candidate

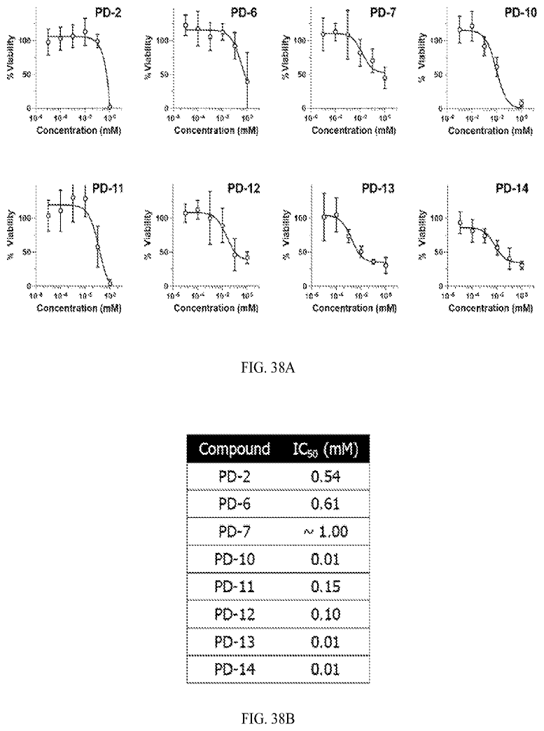

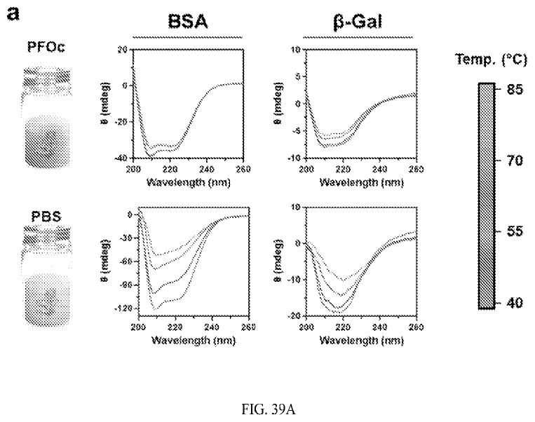

Next, the above compounds were screened for toxicity against the HepG2 human liver hepatocellular cell line (FIGS. 38A-38B). This cell line was chosen as liver is the primary site of metabolism and toxicity for many perfluorinated amphiphiles. PD-7 was the least toxic, with an IC50≈1.0 mM. Thus, PD-7 was prioritized as the lead candidate for further development of thermally stabile protein dispersions, discussed below.

### Evaluation of Thermally Induced Structural Changes of PD-7

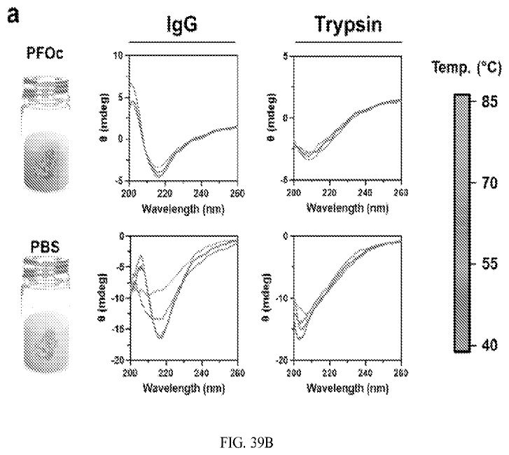

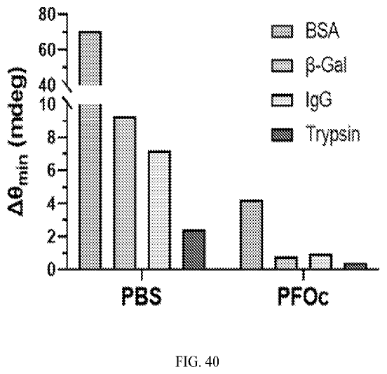

To determine structural integrity of proteins in aqueous and fluorinated environments at elevated temperatures, circular dichroism (CD) was used to probe secondary structure. Proteins were either diluted in PBS (aqueous) or dispersed in PFOc at the same designated concentrations according to protocol described above. For aqueous samples, dissolved proteins were heated between 40 °C to 90 °C, and their corresponding CD spectra at each temperature was measured. For PFOc samples, each of the PFOc dispersed proteins were subjected to heat treatment at 40, 55, 70, and 85 °C for 15 minutes. After that, dispersed proteins were eluted into PBS before having their CD spectra taken. Data is shown by representative ellipticity at each temperature depicted by distinct colors. Results in FIGS. 39A-39B and FIG. 40 show minimal changes in secondary structure for the four model test proteins BSA, β-Gal, IgG and trypsin, when dispersed in PFOc using PD-7 and heated up to 85 °C. Conversely, nearly all the saline control formulations showed denaturation under the tested temperature range, as demonstrated by a significant loss of β-sheet (212 nm) and α-helical (208 & 222 nm) canonical minima. The notable exception was trypsin, which showed a minimal change in structure between 40 °C and 85 °C.

While these results are encouraging, even small changes in protein structure can have significant consequences on bioactivity. Retention of protein activity following elevated temperature exposure was demonstrated using enzymatic conversion and intrinsic fluorescence. A group of enzyme proteins, β-Gal and Trypsin was chosen for this study. β-Gal and Trypsin were diluted in PBS or dispersed in PFOc at 1 μM and 10 μM, respectively. Next, a subset of samples was subjected to elevated temperature condition (90 °C) well beyond their native denaturation temperature for 30 minutes. After that, samples were allowed to cool down to room temperature before retrieval. For PFOc samples, proteins were extracted into PBS before performing the ensuing assays. To determine the enzymatic activity, an equal volume of protein samples was added to their substrates dissolved in PBS (4 mg/mL ONPG for β-Gal or 1 mg/mL BAEE for Trypsin). After incubation, ONPG and BAEE substrate conversions were measured by taking absorbance using a microplate reader at 420 nm or 400 nm, respectively. In case of intrinsic fluorescence, GFP was similarly prepared, and heat treated. After GFP samples were retrieved, fluorescent intensity was taken at λₑₓ=490 nm, λₑₘ=520 nm. The change in protein activity was presented by normalizing to spectrometric readings of each protein dissolved in PBS without being subjected to heat (room temperature). As expected, both proteins in saline lost>90% of their bioactivity after heating, while no statistically significant change in activity of the PFOc dispersed proteins was detected under the same conditions. This supports the assertion that the fluorous coating and solvent environments, together, serve to constrain the conformational flexibility of the dispersed protein and, as a result, increase its melting temperature.

### Biophysical Mechanisms of PD-7:Protein Interaction

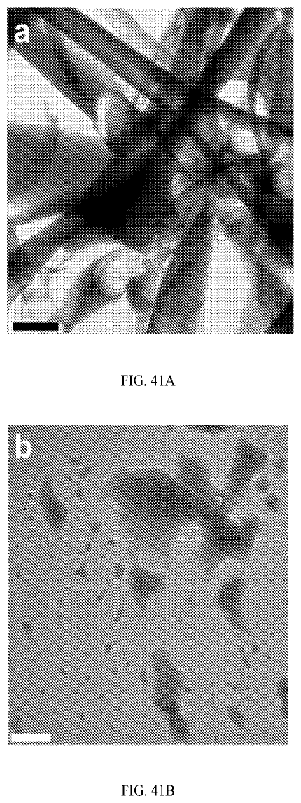

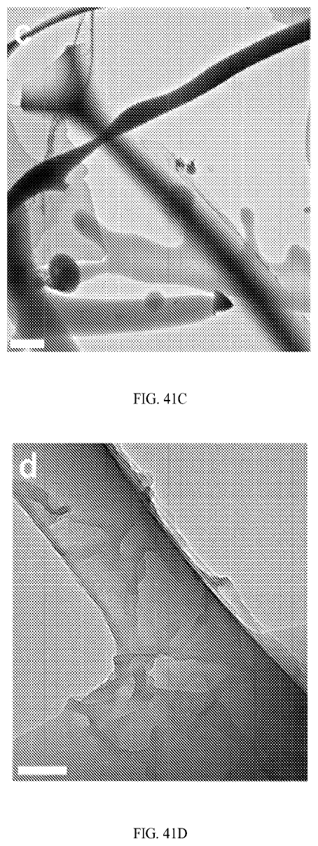

Next, a BSA model was used to gain a deeper understanding of the biophysical mechanisms of PD-7:protein interaction and associated stabilizing effects. To prepare protein sample, model BSA protein was diluted in water at 1 μM or dispersed (10 μM) in PFOc. 5 μL of prepared samples was added to TEM copper grid and visualized by TEM imaging. TEM demonstrated that, without the PD-7 dispersant, BSA aggregates into amorphous structures when dispersed into PFOc (FIG. 41A). Conversely, PD-7, on its own, showed an oil like assembled morphology when dissolved in PFOc (FIG. 41B). When PD-7 and BSA are combined in PFOc, however, dispersed proteinaceous fibrils are observed (FIG. 41C), where the fiber surface is coated by the oily PD-7 dispersant (FIG. 41D). It is hypothesized that this coating provides a fluorophilic canopy to the surface of protein assemblies that potentiates their solubilization by the PFOc solvent.

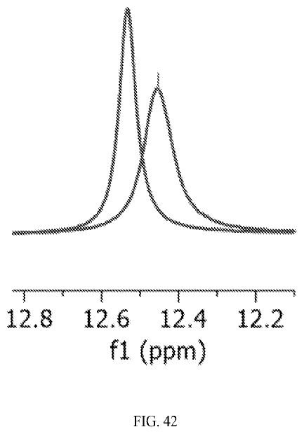

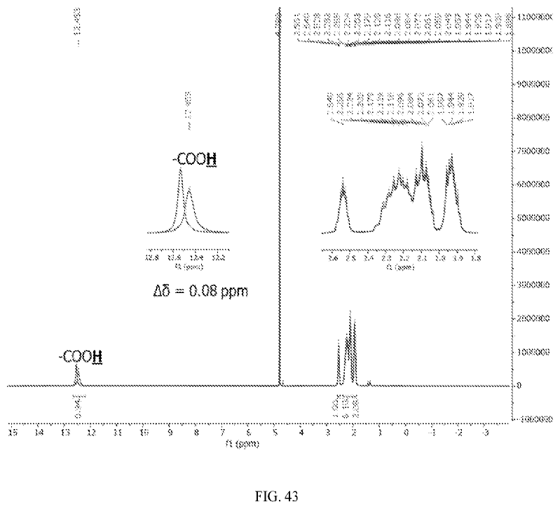

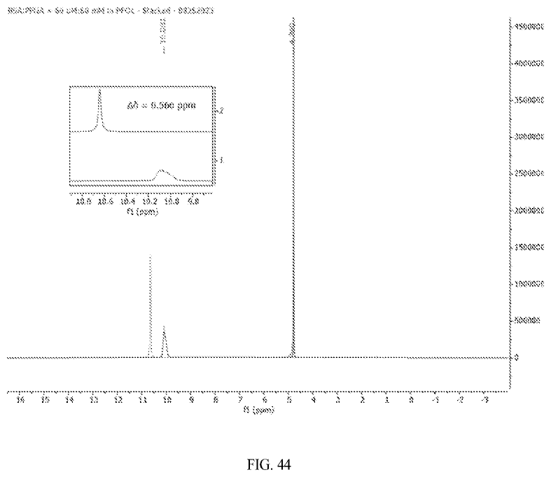

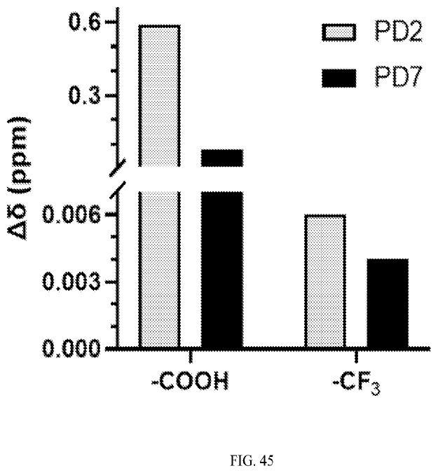

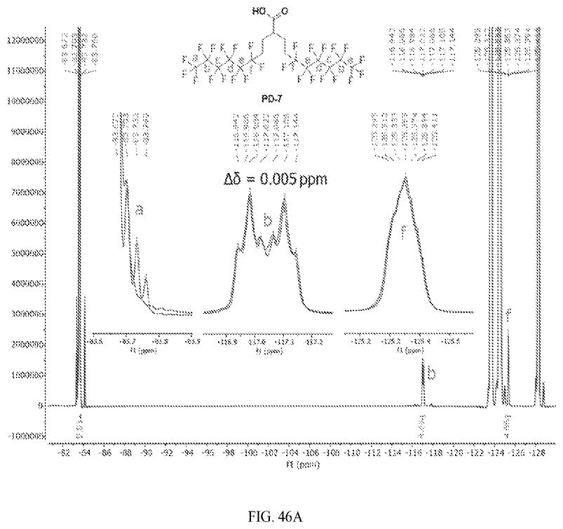

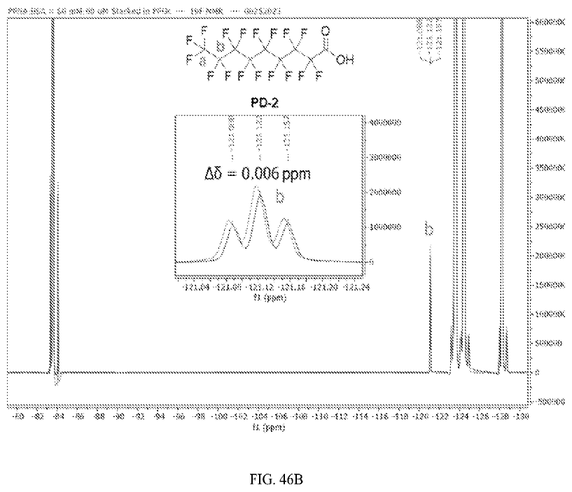

1H NMR and 19F NMR spectroscopy were employed to examine the biophysical interactions between BSA and PD-7 in the fluorous solvent. 60 mM PFNA solution in PFOc was aliquoted into lyophilized BSA stocks to achieve 1000:1 PFNA/BSA molar ratio then transferred to thin wall precision tube with Norell Coaxial inserts containing D2O as locking solvent. NMR spectra were collected on a Bruker NEO-400, equipped with a double resonance broadband observe iProbe (capable of automatic tuning and matching) for 1H and 19F nuclei observation. These proton NMR experiments showed a 0.08 ppm downfield shift of PD-7's —COOH hydrogen in the presence of BSA (FIGS. 42-43), suggesting it weakly hydrogen bonds with the solvent exposed backbone and amino acid sidechains of BSA. In contrast, PD-2, the first-generation dispersant, demonstrated strong hydrogen bonding with BSA, as exemplified by a —COOH downfield shift of 0.56 ppm (FIG. 44). This is a 7-fold change in chemical shift relative to PD-7:BSA (FIG. 45). Conversely, 19F NMR showed a negligible change in the chemical environment of CF3 groups for both dispersants in the presence of BSA (PD-7 Δδ=0.005 ppm and PD-2 Δδ=0.006 ppm) (FIG. 45, FIGS. 46A-46B).

Taken together, the data indicates that hydrogen bonding between the dispersant carboxylic head group and the protein surface is responsible for the coating phenomenon observed in TEM (FIGS. 41A-41D), while the fluorinated tail(s) is/are extended into the bulk solution to promote solvation of the complex by PFOc. The stronger hydrogen-bonding interactions of PD-2 with BSA, relative to PD-7, contradict the dispersion performance of these compounds, where prior studies showed PD-7 was a superior dispersant.

While computational studies presented below help to explain this paradoxical finding, it is hypothesized that the weak binding of PD-7 may promote rapid decoupling of the dispersant from the protein surface in a physiologic solution, like blood. This is advantageous because, given the desire for shelf-stable protein formulations that can be directly administered to patients, a dispersant was sought that will remain in the PFOc solution during protein partitioning into physiologic solutions to avoid interfering with the protein's native function. Additionally, retention of the dispersant in the PFOc solvent, which is expected to be renally excreted in patients, will limit bioaccumulation of the fluorochemical in tissues and reduce its potential for toxic side effects.

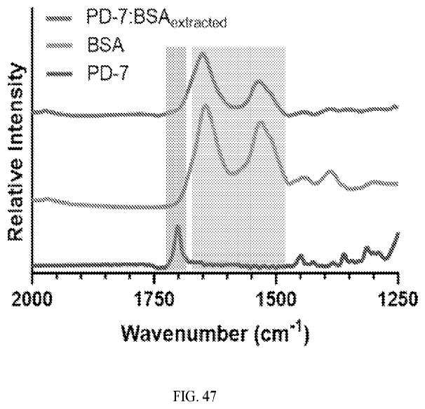

To test this assertion, Fourier transform infrared (FTIR) spectroscopy was performed on BSA samples following extraction of the PD-7 dispersed protein from PFOc into a physiologic buffer. Selected model protein BSA was diluted to 20 PM, and PFNA was diluted to 2 mM in PBS. The intermolecular interactions were initiated by adding an equal amount of BSA and PFNA samples into a centrifuge tube. After 1-hour incubation, samples were frozen at −80 °C, and lyophilize overnight to remove water. FTIR spectrometry was then performed on powdered samples using LN-MCT detector (4000-800 cm-1, backward output). Data is represented by average+−SD of wavenumber among replicates depicting corresponding characteristic bonds. An equal volume of PBS was added to the BSA:PD-7 sample in PFOc, and vortexing the phase separated mixture for ~2 seconds. FTIR spectra of the extracted fraction showed only the presence of BSA's amide-I (1644 cm-1) and II (1531 cm-1) bands, and did not show the C═O stretching spectral feature (1701 cm-1) indicative of PD-7 (FIG. 47).

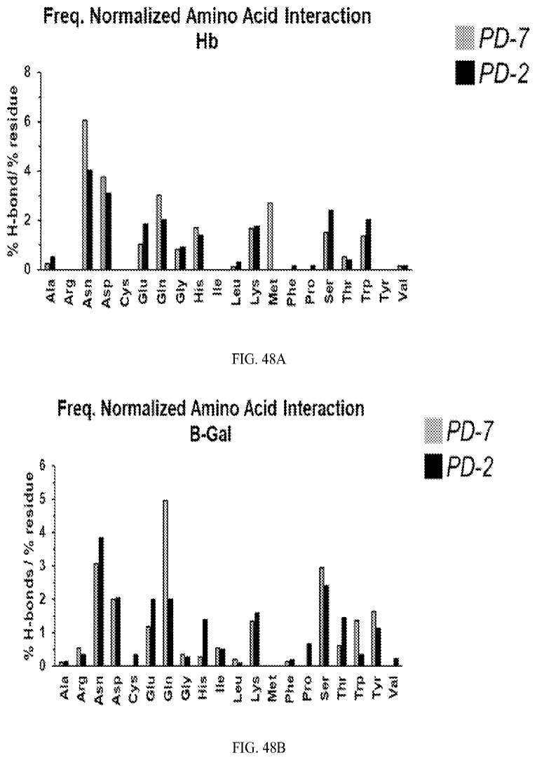

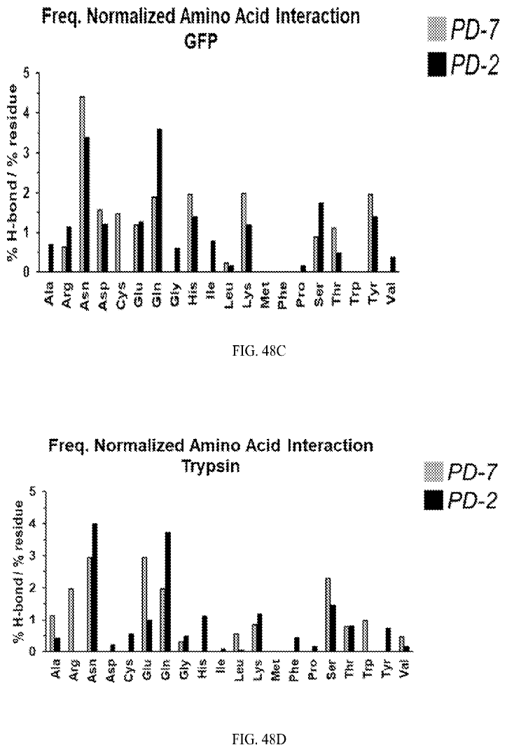

This result supports the assertion that, in an in vivo setting, the PFOc liquid, containing PD-7, would subsequently be cleared in the urine to avoid long-term accumulation fluorinated compounds in tissues. On the contrary, FTIR spectra of PD-2 dispersed BSA samples extracted into PBS showed the presence of C—F stretching vibrations (1500-1000 cm-1) from the dispersant (FIGS. 48A-48D). This indicates that PD-2 co-elutes into the PBS layer, possibly due to remaining coupled to the BSA surface or by forming micelles in the aqueous fraction. In either case, the data suggests that, unlike PD-2, PD-7 successfully balances hydrogen bonding propensity and fluorophilicity to enable its use as a transient masking agent for fluorous protein formulations.

### In Silico Examination of PD-2/PD-7:Protein Molecular Interactions

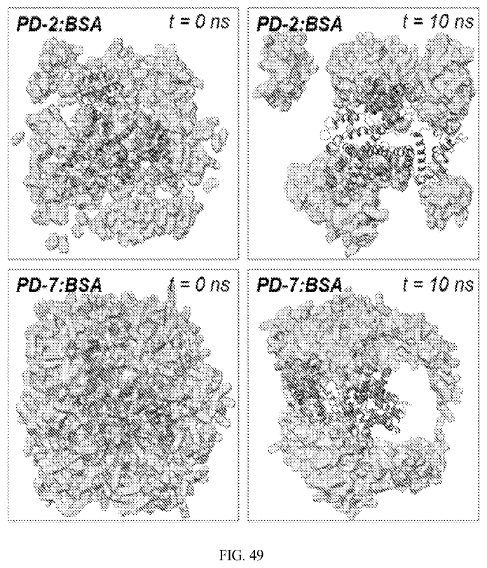

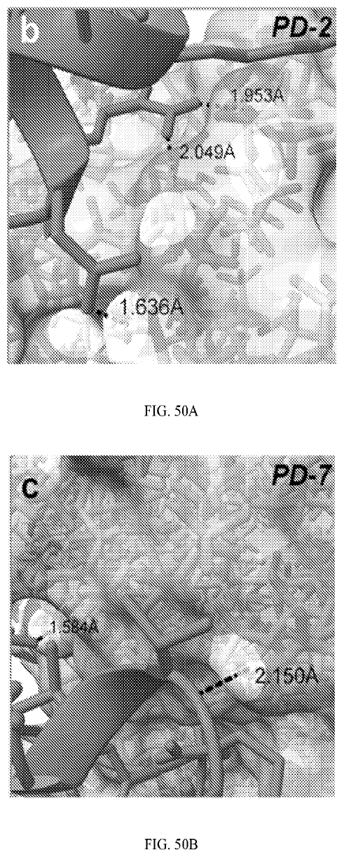

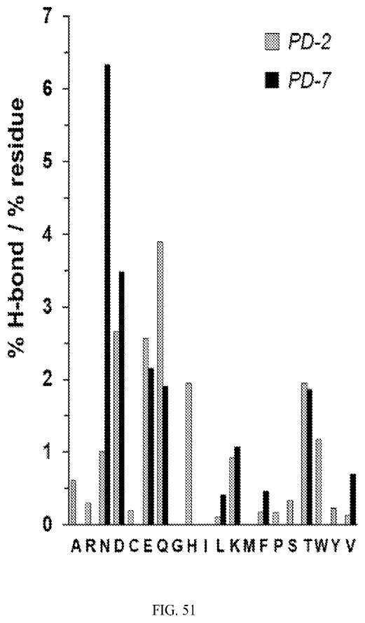

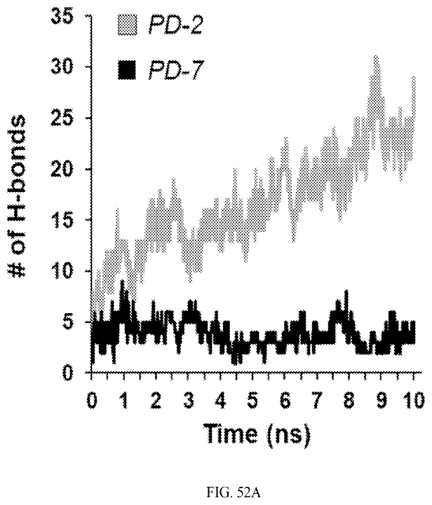

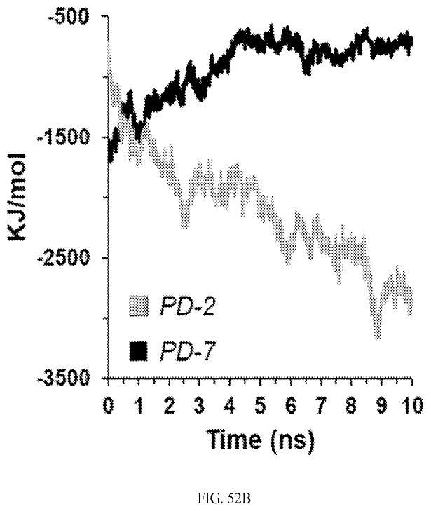

To gain a deeper mechanistic insight into the complexation of the dispersants with protein, simulations of discrete molecular dynamics of PD-2 and PD-7 interactions with BSA (FIG. 49) were performed. The most striking finding from these in silico models was a distinct morphologic difference between the assemblies. Over the 10-nanosecond simulation time, PD-2 organized into discrete clusters that predominantly absorbed solvent exposed loop regions of the protein. In contrast, PD-7 assembled into fibrillar bundles that circumscribed the protein surface, forming a cage-like structure. This agrees with the TEM experiments described above, which showed PD-7 sheets encompassing protein assemblies (FIG. 41D). These analyses also corroborated the ability of PD-2 (FIG. 50A) and PD-7 (FIG. 50B) to form hydrogen bonds with amino acid side chains and backbone of the solvent exposed protein surface, with distances typical for protein-ligand interactions (~1.5 Å-2.0 Å). Plotting the frequency normalized, amino acid-specific, hydrogen bonding propensity of each dispersant showed a general preference for interactions with residues that possess both hydrogen bond donors and acceptors in their side chains (FIG. 51), which include asparagine (N), glutamine (Q), aspartic acid (D) and glutamic acid (E).

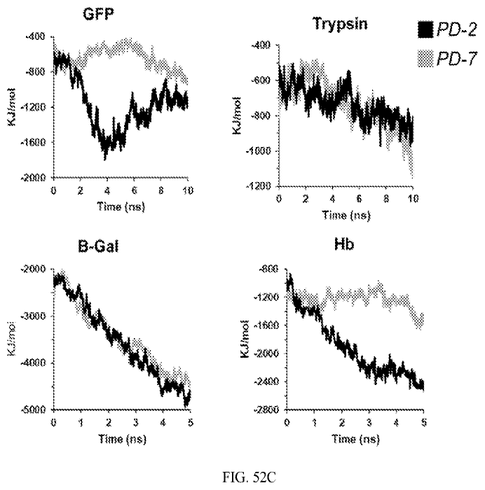

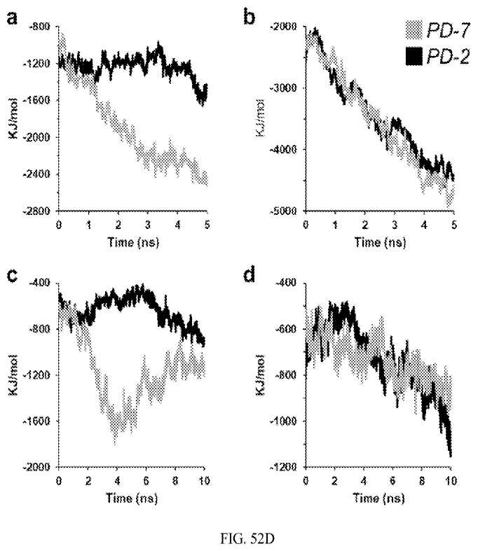

As expected, PD-2 showed a greater number of hydrogen bonds formed with the protein surface (FIG. 52A), and a low predicted docking free energy (FIG. 52B), compared to PD-7 over the simulation period. This corroborates the NMR experimental data (FIGS. 42, 45) described above, and further demonstrates the thermodynamically favored interactions between PD-2 and the protein surface. Analyses of dispersant oligomerization showed a rapid decrease in the free energy of PD-7 as it assembled with itself, whereas PD-2 self-assembly was not favored. Similar findings resulted from analogous simulations with hemoglobin, (3-galactosidase, GFP and trypsin (FIG. 52C-52D).

In sum, the in silico experiments indicate that homo-oligomeric assembly of PD-7 creates laminated bundles that encompass the protein surface more uniformly compared to PD-2, thereby generating a more complete fluorophilic coating that promotes protein solubilization in PFOc. PD-2, in contrast, favors hydrogen bonding with the protein surface, rather than self-assembly, leading to the formation of dispersant islands that leave large areas exposed to the fluorous solvent. Finally, the weak van der Waals interactions between PD-7 and protein surfaces explains the additional benefit of rapid decoupling in ionic solutions observed during FTIR experiments (FIG. 47).

Evaluating PFOc Protein Formulation Resilience Against Contaminants Aqueous protein formulations can be compromised by contaminating pathogens, or the biologic inactivated by exposure to environmental disinfectants and acids. In the context of bacterial and fungal contamination, it was hypothesized that removal of the water solvent required for microorganismal survival should make the PFOc protein formulations intrinsically sterile. A hypodermic needle was streaked across a lawn of the human bacterial pathogens E. coli, P. aeruginosa, K. pneumoniae or Methicillin-resistant S. aureus (MRSA), as well as the human fungal pathogen C. albicans prepared on agar. The contaminated needle was then submerged into BSA protein formulations prepared in either PBS or PFOc solvents. Contaminated liquid formulations were incubated at 37 °C overnight and replated onto agar plates to assess growth.

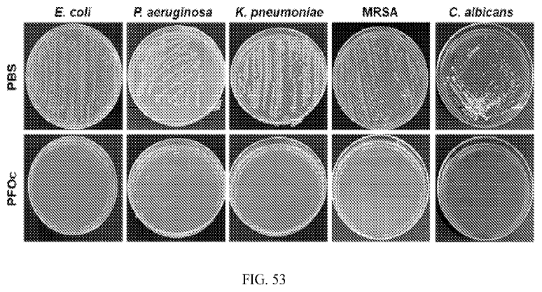

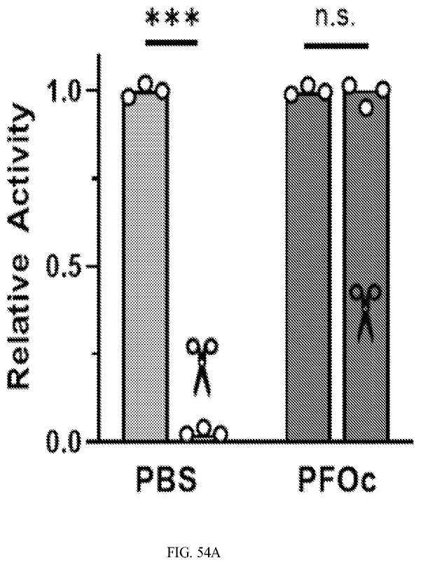

Results in FIG. 53 show, as expected, BSA in PBS was readily contaminated by all five pathogens, as demonstrated by the emergence of viable colonies on the plate. In contrast, PFOc samples remained sterile, despite the high pathogen concentrations inoculated into the samples. A possible explanation for this is that contaminating pathogens dehydrate when submerged in the non-aqueous PFOc formulation, leading to their elimination.

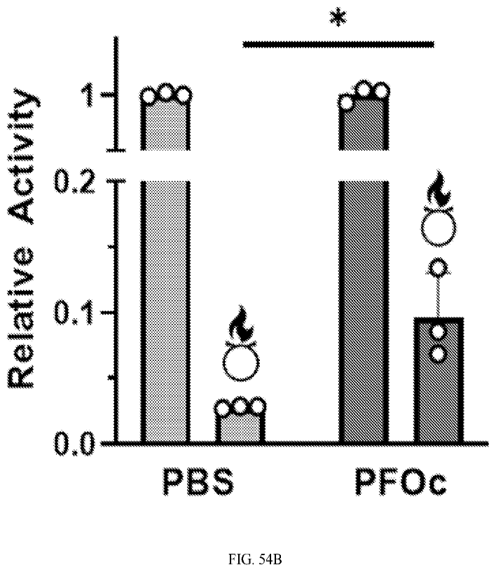

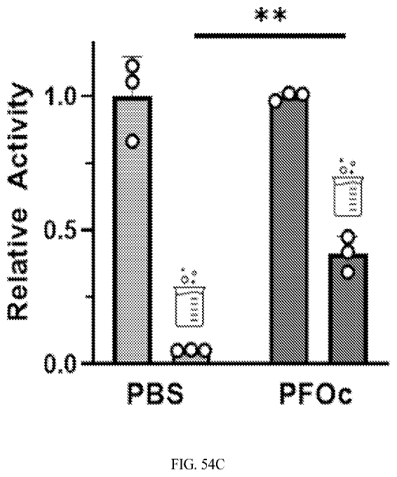

In addition to microorganisms, protein formulations can be compromised by incidental contact with environmental proteases, oxidizing cleaners, and acidic disinfectants. To empirically model these conditions, aliquots of either proteinase K, a chlorine and sodium hydroxide mixture (bleach), or hydrochloric acid, were added to PFOc and PBS protein samples (FIG. 54A-54C).

First, resistance against common denaturation enzymes produced by organisms was demonstrated. β-Gal bioactivity was determined after exposure to potent proteolytic enzyme, proteinase K. Lyophilized β-Gal (1 μM) and proteinase K (10 μM) was either diluted in PBS or dispersed in PFOc. An equal volumetric amount of β-Gal and proteinase K stocks were mixed and incubated together for 24 hours. The remaining active β-Gal was evaluated by adding ONPG substrate diluted in PBS at 4 mg/mL directly to the samples. This action eluted β-Gal from PFOc solvent and immediately starting ONPG conversion without further β-Gal disruption from residue proteinase K that was also eluted into aqueous buffer. The enzymatic activity was calculated from measuring absorbance (420 nm) after 15-minute conversion. Data was normalized to β-Gal activity that was carried by the same solvent, without proteinase K. In the presence of proteinase K, β-Gal dissolved in PBS was completely inactivated, while there was no statistically significant change in activity for PFOc formulations under similar conditions (FIG. 54A). A likely explanation for this is that the proteinase K protein is also coated by the PD-7 additive after addition to the PFOc solvent, leading to its segregation from the co-dispersed β-Gal protein.

Next, resistance against oxidizer contaminant was demonstrated in β-Gal exposed to a mixture of sodium hypochlorite (bleach). Here, stock of lyophilized β-Gal was prepared then dispersed in PBS or PFOc. A small volumetric quantity (1% v/v) of 10% bleach was added to the protein samples. Immediately, an equal volume of PBS containing 4 mg/mL ONPG substrate was added to the sample and incubated for 5 minutes to allow for complete substrate conversion. Last, absorbance was taken at 420 nm on the eluted samples, and relative activity was calculated by normalizing absorbance of converted ONPG in oxidizer-treated samples against their corresponding untreated group. Resistance against acid contaminant was demonstrated in β-Gal exposed to a strong acid, hydrochloric acid (HCl). Here, stock of lyophilized B-Gal was prepared then dispersed in PBS or PFOc. A small volumetric quantity (4% v/v) of 0.1 M HCl was added to the protein samples. After 30 seconds incubation, PBS containing 4 mg/mL ONPG was added to each of the samples to elute and start substrate conversion. After 3-minute conversion, the eluted samples were retrieved, and absorbance was read at 420 nm. Data is represented by normalizing acid-treated samples to the corresponding untreated group.

In sum, the fluorous formulations were found to be less resistant to chemical denaturants (FIGS. 54B-54C). While the activity of β-Gal in PFOc, and then treated with an oxidizer (FIG. 54B) or acid (FIG. 54C), was statistically higher than that of PBS controls, both conditions lead to a ≥50% loss in functionality after a few minutes of incubation. It should be noted that, in a typical incidental exposure in a healthcare setting, protein solutions would be contaminated by noxious vapors, rather than directly spiked with a solution of the denaturant as done in an experimental setting. Despite these severe conditions, PFOc samples still lead to an improvement in protein stability relative to standard saline control solutions.

### Evaluation of Pharmacokinetic Parameters and Acute Toxicological Impact of PD-7-Mediated Protein Formulations

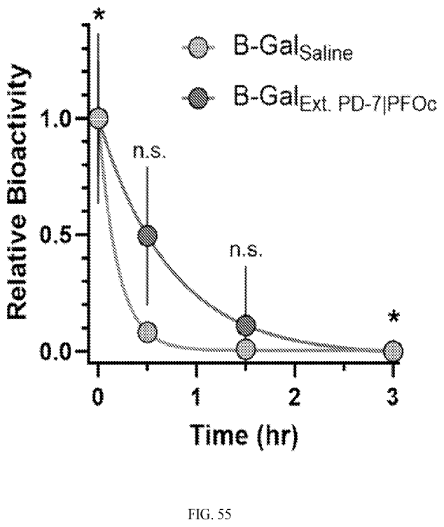

To test whether PD-7 coating and PFOc dispersion influences the pharmacokinetic properties of formulated proteins, f-Gal formulations were intravenously administered to C57BL/6 mice and monitored time dependent serum bioavailability (FIG. 55). This experiment utilized a fluorescent conversion substrate to monitor the amount of functional protein in serum, thereby investigating changes to both bioavailability and bioactivity. Due to the large solution volumes administered (150 μL, ~15% of mouse blood volume), PFOc dispersed proteins were extracted into sterile saline before injection to avoid hyponatremia. Future applications in larger mammals would allow direct injection of the fluorous dispersion, without pre-extraction, due to the increased blood volumes and bio-inert nature of most perfluorocarbon solvents. Results in FIG. 55 shows that the serum half-life of β-Gal from PFOc formulations was slightly extended (t_{1/2}=0.50 hr) relative to the native β-Gal administered in saline (t_{1/2}=0.12 hr). However, variance in the data yielded statistical significance only at the t=0 hr and t=3 hr time points.

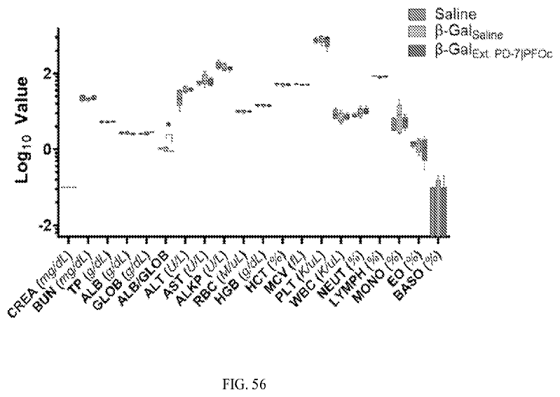

Finally, to assess the potential for acute toxicity of the fluorous dispersant, serologic chemistry analysis and histology was performed 24 hours after treating mice with β-Gal delivered from saline or PFOc extractions. Hematologic, renal, and hepatic blood toxicity markers showed a statistically significant reduction in the albumin/globulin (ALB/GLOB) ratio between β-Gal delivered from PFOc and saline (FIG. 56). Reduction of this marker can indicate a transient impairment of kidney and/or liver function. However, the absence of statistically significant changes in the other renal (e.g., CREA, BUN, TP, etc.) and hepatic (e.g., ALT, AST, ALKP, etc.) markers suggests the ALB/GLOB finding is not likely to be clinically meaningful. This is further corroborated by histologic analysis of vital organs, which did not identify pathologic signs of necrosis, cellular infiltration, inflammation, or hemorrhage in kidney, liver, lung, and spleen tissue. Collectively, these results suggest the perfluorinated reagents used in the above described protein formulations are unlikely to induce acute toxic side effects.

### Conclusion

Results disclosed herein (e.g., Examples 1 and 3) demonstrated that proteins coated by the amphiphilic perfluorinated dispersant PD-2 (PFNA), and dissolved in non-aqueous fluorous solvents, restricted the conformational plasticity of proteins and yielded biologics that were structured and functional at temperatures as high as 90 °C. Based on this work, a library of novel dispersants designed to improve dispersion efficiencies and protein-dispersant decoupling rates were synthesized, screened, and validated. The bivalent perfluorinated carboxylic acid PD-7 was validated as a lead second-generation candidate due to its high dispersion efficiencies (>95%) and low cytotoxicity (IC50≈1.0 mM). Subsequent thermostability experiments showed that proteins dispersed into PFOc using PD-7 had near complete retention of bioactivity at temperatures up to 90 °C. A series of biophysical and in silico assays elucidated the mechanistic basis for this thermal stabilization. Finally, in vitro and in vivo experiments show that fluorous-dispersed proteins remain sterile after intentional contamination by bacterial and fungal pathogens, which require aqueous solvents for survival, and display equivalent pharmacologic properties, bioactivity and safety profiles compared to standard saline protein formulations in mice. These findings show that PD-7 is a scalable and clinically relevant protein coating technology that, when paired with intrinsically sterile fluorous solvents, can produce a novel storage paradigm to generate extremophilic protein formulations.

1. A composition comprising a fluorinated solvent, a dispersant compound, and a protein of interest, wherein:
the fluorinated solvent is selected from the group consisting of perfluoropentane, perfluorohexane, perfluoroheptane, perfluorooctane, perfluoromethylcyclohexane, perfluoro-1,3-dimethylcyclohexane, and perfluorodecalin;
the dispersant compound is perfluorinated and interacts with the protein of interest; and
the protein of interest is dissolved in the fluorinated solvent.

2. (canceled)

3. The composition of claim 1, wherein the fluorinated solvent is perfluorohexane.

4. The composition of claim 1, wherein the fluorinated solvent is perfluorooctane.

5. (canceled)

6. The composition of claim 1, wherein the dispersant Compound comprises Formula II: 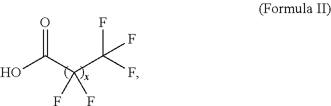
wherein x is 6, 7, 8, 9, 10, 12, or 16.

7. The composition of claim 6, wherein x is 7 (perfluoronanonic acid).

8. The composition of claim 1, wherein the dispersant compound comprises Formula III:

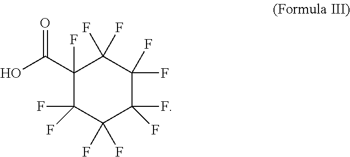 [SMILES: O=C(O)C1(F)C(F)(F)C(F)(F)C(F)(F)C(F)(F)C1(F)F]

9. The composition of claim 1, wherein the dispersant compound comprises Formula V: 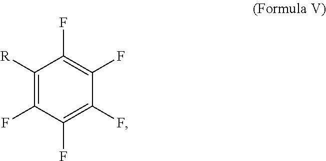
wherein R is OH or COH.

10. The composition of claim 1, wherein the dispersant compound comprises Formula VI: 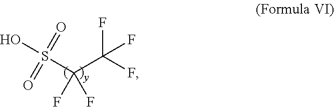
wherein y is 7.

11. The composition of claim 1, wherein the dispersant compound comprises Formula VIII: 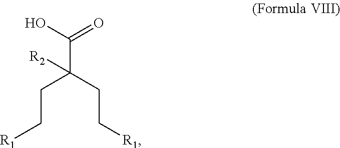
wherein R₁ is C₆F₁₃, C₅F₁₇, or C₄F₉, and
wherein R₂ is CN, H, or COOH.

12. The composition of claim 11, wherein R₁ is C₆F₁₃ and R₂ is H.

13. The composition of claim 1, wherein the dispersant compound interacts with the protein of interest via hydrogen bonding with nitrogen donors present in the protein backbone and amino acid side chains of the protein of interest.

14. The composition of claim 1, wherein the protein of interest comprises a non-native secondary structure.

15. The composition of claim 1, wherein the fluorinated solvent is perfluorohexane and the dispersant compound is perfluoronanonic acid.

16. The composition of claim 1, wherein the fluorinated solvent is perfluorooctane and the dispersant compound comprises Formula VIII: 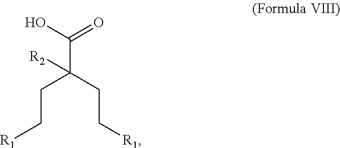
wherein R₁ is C₆F₁₃ and R₂ is H.

17. A method of dissolving a protein of interest in a fluorinated solvent, the method comprising:
contacting the protein of interest with a dispersant compound, and
dissolving the protein of interest and dispersant compound in the fluorinated solvent;
wherein the fluorinated solvent is selected from the group consisting of perfluoropentane, perfluorohexane, perfluoroheptane, perfluorooctane, perfluoromethylcyclohexane, perfluoro-1,3-dimethylcyclohexane, and perfluorodecalin; and
wherein the dispersant compound is perfluorinated.

18. (canceled)

19. The method of claim 17, wherein the fluorinated solvent is perfluorohexane.

20. The method of claim 17, wherein the fluorinated solvent is perfluorooctane.

21-28. (canceled)

29. The method of claim 17, wherein the dispersant compound interacts with the protein of interest via hydrogen bonding with nitrogen donors present in the protein backbone and amino acid side chains of the protein of interest.

30. (canceled)

31. The method of claim 17, wherein the fluorinated solvent is perfluorohexane and the dispersant compound is perfluoronanonic acid.

32. The method of claim 17, wherein the fluorinated solvent is perfluorooctane and the dispersant compound comprises Formula VIII: 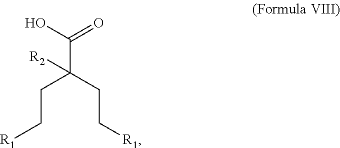
wherein R₁ is C₈F₁₃ and R₂ is H.
